(19) United States
(12) Patent Application Publication
(10) Pub. No.: US 2012/0010094 A1
(43) Pub. Date: Jan. 12, 2012

(54) DETECTING AND MONITORING LEFT VENTRICULAR HYPERTROPHY AND CONGESTIVE HEART FAILURE BY PROFILING BIOMARKERS

(75) Inventors: Francis G. Spinale, Chareleston, SC (US); Michael R. Zile, Charleston, SC (US); Robert E. Stroud, Mt. Pleasant, SC (US)

(73) Assignee: MUSC Foundation for Research Development, Charleston, SC (US)

(21) Appl. No.: 13/128,200

(22) PCT Filed: Nov. 4, 2009
(86) PCT No.: PCT/US09/63309

§ 371 Date: Sep. 22, 2011

Related U.S. Application Data
(60) Provisional application No. 61/112,135, filed on Nov. 6, 2008.

(51) Int. Cl. C40B 30/04; G01N 33/566; G01N 33/573; C12Q 1/37

(52) U.S. Cl. 506/9; 435/23; 435/7.92; 435/7.4; 436/501

(57) ABSTRACT

Disclosed herein are methods of detecting, monitoring, and predicting left ventricular hypertrophy, congestive heart failure and related conditions by profiling biomarkers.

### STATEMENT REGARDING FEDERALLY SPONSORED RESEARCH

This invention was made with government support under Grant No. 5R01HL059165-0 awarded by the National Institutes of Health. The government has certain rights in the invention.

### BACKGROUND

Despite significant advancements in high blood pressure (hypertension) medicines and the recognition that hypertension is a significant risk factor for the development of heart failure, this condition remains a major cardiovascular disease in the United States. One particular problem with identifying patients at risk for developing hypertensive heart failure is the lack of a rapid screening test to identify patients that have changes occurring in the heart muscle itself secondary to hypertension. With prolonged hypertension, the muscle mass and size of the heart increases, but this may not occur until later in the disease process. One unique and critical event in the progression to hypertensive heart disease and heart failure is that increased fibrosis occurs within the heart muscle itself. The molecular basis for this change remains unknown.

### BRIEF SUMMARY

In accordance with the purpose of this invention, as embodied and broadly described herein, this invention relates to unique patterns of MMPs/TIMPs and other biomarkers that occur in subjects and patients with developing hypertensive heart failure that were actually predictive of the presence of abnormal heart function-heretofore only possible to identify with expensive and difficult to apply tests. The unique pattern of MMPs/TIMPs and other biomarkers can be used in methods, for example, for the identification of patients at risk of and soon to develop heart failure secondary to hypertension, and in other methods as described elsewhere herein.

Disclosed is a method to, for example, diagnose a subject with left ventricular hypertrophy (LVH, HCM or HOCM), at risk for left ventricular hypertrophy, at risk for congestive heart failure and/or diastolic heart failure, or with congestive heart failure and/or diastolic heart failure. For example, provided is a method of detecting LVH in a subject, comprising identifying a profile of matrix metalloproteinases (MMPs), tissue inhibitors of matrix metalloproteinases (TIMPs), and/or other biomarkers (such as propeptide for collagen I (PINP), propeptide for collagen III (PIIINP), C-telopeptide for type-I collagen (CITP), cardiotrophin, soluble receptor for advanced glycated end products (sRAGE), osteopontin, and N-terminal pro-B-type natriuretic peptide (NTBNP)) from a body fluid of the subject that is associated as described herein with the existence or risk of left ventricular hypertrophy (LVH), congestive heart failure (CHF) and/or diastolic heart failure (DHF). Also provided is a method of, for example, predicting congestive heart failure and/or diastolic heart failure in a subject, comprising identifying a profile of matrix metalloproteinases (MMPs), tissue inhibitors of matrix metalloproteinases (TIMPs), and/or other biomarkers (such as propeptide for collagen I (PINP), propeptide for collagen III (PIIINP), C-telopeptide for type-I collagen (CITP), cardiotrophin, soluble receptor for advanced glycated end products (sRAGE), osteopontin, and N-terminal pro-B-type natriuretic peptide (NTBNP)) from a body fluid of the subject that is associated as described herein with the likely development of congestive heart failure (CHF) and/or diastolic heart failure (DHF).

Also disclosed is a method to, for example, determine the presence or risk of congestive heart failure in a subject, comprising measuring the amount of two or more biomarkers in a body fluid from a subject, wherein the amount of two or more of the two or more biomarkers compared to a reference amount for the biomarker (for example, the amount in a normal subject, the amount in a subject with hypertension, or the amount in a subject with left ventricular hypertrophy) indicates the presence or risk of congestive heart failure in the subject.

Also disclosed is a method to, for example, determine the presence or risk of left ventricular hypertrophy in a subject, comprising measuring the amount of two or more biomarkers in a body fluid from a subject, wherein the amount of two or more of the two or more biomarkers compared to a reference amount for the biomarker (for example, the amount in a normal subject, the amount in a subject with hypertension, or the amount in a subject with left ventricular hypertrophy) indicates the presence or risk of congestive heart failure in the subject.

The two or more biomarkers can be propeptide for collagen I (PINP), propeptide for collagen III (PIIINP), C-telopeptide for type-I collagen (CITP), cardiotrophin, soluble receptor for advanced glycated end products (sRAGE), osteopontin, MMP-2, MMP-3, MMP-7, MMP-8, MMP-9, MMP-13, TIMP-1, TIMP-2, TIMP-4, N-terminal pro-B-type natriuretic peptide (NTBNP), gender, and ethnicity.

At least one of the two or more biomarkers can be PINP, PIIINP, CITP, cardiotrophin, sRAGE, osteopontin, gender, or ethnicity. At least one of the two or more biomarkers can be PINP, PIIINP, CITP, cardiotrophin, sRAGE, osteopontin, gender, or ethnicity. At least one of the two or more biomarkers can be PINP, PIIINP, CITP, cardiotrophin, sRAGE, osteopontin, gender, or ethnicity, and at least one other of the two or more biomarkers can be a MMP or TIMP.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows MMP-13 detectability in reference control with and without hypertension and in LVH with and without chronic heart failure. MMP-13 detectability decreased significantly in LVH patients. *=p<0.05 vs Reference control without Hypertension, #=p<0.05 vs Reference control with Hypertension, Δ=p<0.05 vs LVH without CHF.

FIG. 2A shows relationship between tissue inhibitor of matrix metalloproteinase-1 (TIMP-1) and left ventricular (LV) volume/mass ratio. Higher levels of TIMP-1 were associated with lower values of LV volume/mass ratio indicating more pronounced concentric remodeling. r=−0.56, p<0.05. FIG. 2B shows relationship between tissue inhibitor of matrix metalloproteinase-1 (TIMP-1) and Tissue Doppler imaging (TDI) rapid filling wave (E′). Higher levels of TIMP-1 were associated with lower values of E′ indicating slower LV diastolic relaxation rate. r=−0.41, p<0.05.

FIG. 3 shows structure and function of normal heart compared to heart with diastolic heart failure.

FIGS. 4A-4F show results of echocardiography and MMP-9, MMP-2, and TIMP-1 plasma measurements for controls with and without hypertension (HTN) and subjects with ventricular hypertrophy with and without congestive heart failure (CHF).

FIG. 5 shows Tissue Doppler imaging (TDI) rapid filling wave (E′) relative to TIMP-1 levels.

FIG. 6 shows plasma MMP-13 levels in controls and subjects with left ventricular hypertrophy.

FIG. 7 shows the percentage of patients with or without congestive heart failure and with plasma TIMP-1 levels greater than or less than 1200 ng/ml that also have left ventricular hypertrophy.

FIGS. 8A-8D show calibration curves for IL-6 (A), MMP-9 (B), MMP-13 (C), and TNF-α (D) as determined by multiplex analysis.

FIGS. 9A-9C show algorithm for using MMP and TIMP levels to determine treatment of patients with hypertension. FIG. 9A shows schematic for treatment of patient with documented hypertension scheduled non-emergent clinic visit. FIG. 9B shows schematic for treatment of patient with new onset hypertension non-emergent clinic visit. FIG. 9C shows schematic for treatment of patient presenting with signs or symptoms which might be caused by HF.

FIG. 10 shows a differential profile of biomarkers in patients with established left ventricular hypertrophy when compared to hypertensive patients with no left ventricular hypertrophy.

FIG. 11 shows a flow chart for analysis of 150 subjects which were carefully screened and enrolled in the study after providing informed consent. The patients were bifurcated into a reference control group, with no evidence of hypertension, and a hypertensive subject group as defined by a health care provider, the patient themselves, or current blood pressure medication. The patients in this hypertensive group were further stratified into those subjects with the absence or presence of LVH. The latter was then classified by gender and ethnicity.

FIG. 12 shows a differential profile of biomarkers in patients with hypertension and left ventricular hypertrophy based on the gender of the patients. It can be seen that males had a general increase in MMPs (with the exception of MMP-7) as compared to females.

FIG. 13 shows a differential profile of biomarkers in patients with hypertension and left ventricular hypertrophy based on the ethnicity of the patients. It can be seen that black patients had a significant increase in TIMP-2 as compared to white patients.

FIG. 14 shows a matrix of biomarkers used in combination as indicators of left ventricular hypertrophy.

FIGS. 15A-15C show histograms of the levels of biomarkers in subjects with and without LVH.

### DETAILED DESCRIPTION

The disclosed method and compositions may be understood more readily by reference to the following detailed description of particular embodiments and the Example included therein and to the Figures and their previous and following description.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a peptide is disclosed and discussed and a number of modifications that can be made to a number of molecules including the peptide are discussed, each and every combination and permutation of peptide and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Those skilled in the art will recognize, or be able to ascertain using no more than routine experimentation, many equivalents to the specific embodiments of the method and compositions described herein. Such equivalents are intended to be encompassed by the following claims.

It is understood that the disclosed method and compositions are not limited to the particular methodology, protocols, and reagents described as these can vary. It is also to be understood that the terminology used herein is for the purpose of describing particular embodiments only, and is not intended to limit the scope of the present invention which will be limited only by the appended claims.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification. More specifically, the biomarkers whose amounts are measured can have those measurements taken in any order or simultaneously in any combination.

### DEFINITIONS

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents.

It must be noted that as used herein and in the appended claims, the singular forms “a,” “an,” and “the” include plural reference unless the context clearly dictates otherwise. Thus, for example, reference to “a peptide” includes a plurality of such peptides, reference to “the peptide” is a reference to one or more peptides and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Throughout the description and claims of this specification, the word “comprise” and variations of the word, such as “comprising” and “comprises,” means “including but not limited to,” and is not intended to exclude, for example, other additives, components, integers or steps.

“Subject” includes, but is not limited to, animals, plants, bacteria, viruses, parasites and any other organism or entity that has nucleic acid. The subject may be a vertebrate, more specifically a mammal (e.g., a human, horse, pig, rabbit, dog, sheep, goat, non-human primate, cow, cat, guinea pig or rodent, such as a mouse or rat), a fish, a bird or a reptile or an amphibian. The subject may to an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

As defined herein “sample” refers to any sample obtained from an organism. Examples of biological samples include body fluids and tissue specimens. The source of the sample may be physiological media as blood, serum, plasma, breast milk, pus, tissue scrapings, washings, urine, tissue, such as lymph nodes or the like.

Throughout this application, various publications are referenced. The disclosures of these publications are hereby incorporated by reference into this application both for the specific material for which they are cited and, separately, in their entireties, in order to more fully describe the state of the art to which this pertains. The references disclosed are also individually and specifically incorporated by reference herein for the material contained in them that is discussed in the sentence in which the reference is relied upon.

Disclosed is a method to, for example, diagnose a subject with left ventricular hypertrophy (LVH, HCM or HOCM), at risk for left ventricular hypertrophy, at risk for congestive heart failure and/or diastolic heart failure, or with congestive heart failure and/or diastolic heart failure. For example, provided is a method of detecting LVH in a subject, comprising identifying a profile of matrix metalloproteinases (MMPs), tissue inhibitors of matrix metalloproteinases (TIMPs), and/or other biomarkers (such as propeptide for collagen I (PINP), propeptide for collagen III (PIIINP), C-telopeptide for type-I collagen (CITP), cardiotrophin, soluble receptor for advanced glycated end products (sRAGE), osteopontin, and N-terminal pro-B-type natriuretic peptide (NTBNP)) from a body fluid of the subject that is associated as described herein with the existence or risk of left ventricular hypertrophy (LVH), congestive heart failure (CHF) and/or diastolic heart failure (DHF). Also provided is a method of, for example, predicting congestive heart failure and/or diastolic heart failure in a subject, comprising identifying a profile of matrix metalloproteinases (MMPs), tissue inhibitors of matrix metalloproteinases (TIMPs), and/or other biomarkers (such as propeptide for collagen I (PINP), propeptide for collagen III (PIIINP), C-telopeptide for type-I collagen (CITP), cardiotrophin, soluble receptor for advanced glycated end products (sRAGE), osteopontin, and N-terminal pro-B-type natriuretic peptide (NTBNP)) from a body fluid of the subject that is associated as described herein with the likely development of congestive heart failure (CHF) and/or diastolic heart failure (DHF).

Also disclosed is a method to, for example, determine the presence or risk of congestive heart failure in a subject, comprising measuring the amount of two or more biomarkers in a body fluid from a subject, wherein the amount of two or more of the two or more biomarkers compared to a reference amount for the biomarker (for example, the amount in a normal subject, the amount in a subject with hypertension, or the amount in a subject with left ventricular hypertrophy) indicates the presence or risk of congestive heart failure in the subject.

Also disclosed is a method to, for example, determine the presence or risk of left ventricular hypertrophy in a subject, comprising measuring the amount of two or more biomarkers in a body fluid from a subject, wherein the amount of two or more of the two or more biomarkers compared to a reference amount for the biomarker (for example, the amount in a normal subject, the amount in a subject with hypertension, or the amount in a subject with left ventricular hypertrophy) indicates the presence or risk of congestive heart failure in the subject.

The two or more biomarkers can be propeptide for collagen I (PINP), propeptide for collagen III (PIIINP), C-telopeptide for type-I collagen (CITP), cardiotrophin, soluble receptor for advanced glycated end products (sRAGE), osteopontin, MMP-2, MMP-3, MMP-7, MMP-8, MMP-9, MMP-13, TIMP-1, TIMP-2, TIMP-4, N-terminal pro-B-type natriuretic peptide (NTBNP), gender, and ethnicity.

At least one of the two or more biomarkers can be PINP, PIIINP, CITP, cardiotrophin, sRAGE, osteopontin, gender, or ethnicity. At least one of the two or more biomarkers can be PINP, PIIINP, CITP, cardiotrophin, sRAGE, osteopontin, gender, or ethnicity. At least one of the two or more biomarkers can be PINP, PIIINP, CITP, cardiotrophin, sRAGE, osteopontin, gender, or ethnicity, and at least one other of the two or more biomarkers can be a MMP or TIMP.

An amount of PIIINP that is greater than the reference amount can indicate the presence or risk of congestive heart failure in the subject. An amount of osteopontin that is greater than the reference amount can indicate the presence or risk of congestive heart failure in the subject. An amount of cardiotrophin that is less than the reference amount can indicate the presence or risk of congestive heart failure in the subject. An amount of sRAGE that is less than the reference amount can indicate the presence or risk of congestive heart failure in the subject. An amount of CITP that is greater than the reference amount can indicate the presence or risk of congestive heart failure in the subject. An increase in the ratio of PIIINP to PINP compared to a reference ratio can indicate the presence or risk of congestive heart failure in the subject. An increase in the ratio of PIIINP to CITP compared to a reference ratio can indicate the presence or risk of congestive heart failure in the subject. An amount of MMP-2 that is greater than the reference amount can indicate the presence or risk of congestive heart failure in the subject. An amount of MMP-3 that is less than the reference amount can indicate the presence or risk of congestive heart failure in the subject. An amount of MMP-7 that is greater than the reference amount can indicate the presence or risk of congestive heart failure in the subject. An amount of MMP-8 that is greater than the reference amount can indicate the presence or risk of congestive heart failure in the subject. An amount of MMP-9 that is greater than the reference amount can indicate the presence or risk of congestive heart failure in the subject. An amount of less than 10 ng/mL of MMP-13 can indicate the presence or risk of congestive heart failure in the subject. An amount of TIMP-1 that is greater than the reference amount can indicate the presence or risk of congestive heart failure in the subject. An amount of TIMP-2 that is less than the reference amount can indicate the presence or risk of congestive heart failure in the subject. An amount of TIMP-4 that is greater than the reference amount can indicate the presence or risk of congestive heart failure in the subject. A reduction in the ratio of MMP-9 to TIMP-1 compared to a reference ratio can indicate the presence or risk of congestive heart failure in the subject. A reduction in the ratio of MMP-9 to TIMP-2 compared to a reference ratio can indicate the presence or risk of congestive heart failure in the subject. A reduction in the ratio of MMP-9 to TIMP-4 compared to a reference ratio can indicate the presence or risk of congestive heart failure in the subject. An amount of NTBNP that is greater than the reference amount can indicate the presence or risk of congestive heart failure in the subject.

### Materials

### A. Biomarkers

Disclosed herein are biomarkers that can be used in various combinations as described herein in the disclosed methods and as indicators of, for example, left ventricular hypertrophy, risk of left ventricular hypertrophy, risk of development of left ventricular hypertrophy, congestive heart failure, risk of congestive heart failure, risk of development of congestive heart failure, diastolic heart failure, risk of diastolic heart failure, and/or risk of development of diastolic heart failure. A biomarker can be any molecule, ratio of molecules, characteristic or phenotype of a subject. For example, useful biomarkers include matrix metalloproteinases (MMPs), tissue inhibitor of metalloproteinases (TIMPs), propeptide for collagen I (PINP), propeptide for collagen III (PIIINP), C-telopeptide for type-I collagen (CITP), cardiotrophin, soluble receptor for advanced glycated end products (sRAGE), osteopontin, N-terminal pro-B-type natriuretic peptide (NTBNP), ratios of biomarkers, gender, and ethnicity.

1. MMPs

Matrix metalloproteinases (MMPs) are zinc-dependent endopeptidases; other family members are adamalysins, serralysins, and astacins. The MMPs belong to a larger family of proteases known as the metzincin superfamily.

The MMPs share a common domain structure. The three common domains are the pro-peptide, the catalytic domain and the haemopexin-like C-terminal domain which is linked to the catalytic domain by a flexible hinge region.

The MMPs are initially synthesized as inactive zymogens with a pro-peptide domain that must be removed before the enzyme is active. The pro-peptide domain is part of “cysteine switch” this contains a conserved cysteine residue which interacts with the zinc in the active site and prevents binding and cleavage of the substrate keeping the enzyme in an inactive form. In the majority of the MMPs the cysteine residue is in the conserved sequence PRCGxPD. Some MMPs have a prohormone convertase cleavage site (Furin-like) as part of this domain which when cleaved activates the enzyme. MMP-23A and MMP-23B include a transmembrane segment in this domain (PMID 10945999).

X-ray crystallographic structures of several MMP catalytic domains have shown that this domain is an oblate sphere measuring 35×30×30 Å (3.5×3×3 nm). The active site is a 20 Å (2 nm) groove that runs across the catalytic domain. In the part of the catalytic domain forming the active site there is a catalytically important Zn2+ ion, which is bound by three histidine residues found in the conserved sequence HExxHxxGxxH. Hence, this sequence is a zinc-binding motif.

The gelatinases, such as MMP-2, incorporate Fibronectin type II modules inserted immediately before in the zinc-binding motif in the catalytic domain (PMID 12486137).

The catalytic domain is connected to the C-terminal domain by a flexible hinge or linker region. This is up to 75 amino acids long, and has no determinable structure.

The C-terminal domain has structural similarities to the serum protein haemopexin. It has a four bladed β-propeller structure. β-propeller structures provide a large flat surface which is thought to be involved in protein-protein interactions. This determines substrate specificity and is the site for interaction with TIMPs. The haemopexin-like domain is absent in MMP-7, MMP-23, MMP-26 and the plant and nematode. MT-MMPs are anchored to the plasma membrane, through this domain and some of these have cytoplasmic domains.

The MMPs can be subdivided in different ways. Use of bioinformatic methods to compare the primary sequences of the MMPs indicates the following evolutionary groupings of the MMPs: MMP-19; MMPs 11, 14, 15, 16 and 17; MMP-2 and MMP-9; all the other MMPs.

Analysis of the catalytic domains in isolation indicates that the catalytic domains evolved further once the major groups had differentiated, as is also indicated by the substrate specificities of the enzymes. The most commonly used groupings (by researchers in MMP biology) are based partly on historical assessment of the substrate specificity of the MMP and partly on the cellular localization of the MMP. These groups are the collagenases, the gelatinases, the stromelysins, and the membrane type MMPs (MT-MMPs). It is becoming increasingly clear that these divisions are somewhat artificial as there are a number of MMPs that do not fit into any of the traditional groups.

The collagenases are capable of degrading triple-helical fibrillar collagens into distinctive ¾ and ¼ fragments. These collagens are the major components of bone and cartilage, and MMPs are the only known mammalian enzymes capable of degrading them. Traditionally, the collagenases are: MMP-1 (Interstitial collagenase), MMP-8 (Neutrophil collagenase), MMP-13 (Collagenase 3), MMP-18 (Collagenase 4, xco14, xenopus collagenase. No known human orthologue), MMP-14 (MT1-MMP) has also been shown to cleave fibrillar collagen, and more controversially there is evidence that MMP-2 is capable of collagenolysis.

The stromelysins display a broad ability to cleave extracellular matrix proteins but are unable to cleave the triple-helical fibrillar collagens. The three canonical members of this group are: MMP-3 (Stromelysin 1), MMP-10 (Stromelysin 2), and MMP-11 (Stromelysin 3). MMP-11 shows more similarity to the MT-MMPs, is convertase-activatable and is secreted therefore usually associated to convertase-activatable MMPs.

The matrilysins include MMP-7 (Matrilysin, PUMP) and MMP-26 (Matrilysin-2, endometase).

The main substrates of gelatinasese are type IV collagen and gelatin, and these enzymes are distinguished by the presence of an additional domain inserted into the catalytic domain. This gelatin-binding region is positioned immediately before the zinc binding motif, and forms a separate folding unit which does not disrupt the structure of the catalytic domain. The two members of this sub-group are: MMP-2 (72 kDa gelatinase, gelatinase-A) and MMP-9 (92 kDa gelatinase, gelatinase-B).

The secreted MMPs include MMP-11 (Stromelysin 3), MMP-21 (X-MMP), and MMP-28 (Epilysin).

The membrane-bound MMPs include: the type-II transmembrane cysteine array MMP-23, the glycosyl phosphatidylinositol-attached MMPs 17 and 25 (MT4-MMP and MT6-MMP respectively), and the type-I transmembrane MMPs 14, 15, 16, 24 (MT1-MMP, MT2-MMP, MT3-MMP, and MT5-MMP respectively).

All 6 MT-MMPs have a furin cleavage site in the pro-peptide, which is a feature also shared by MMP-11.

Other MMPs include MMP-12 (Macrophage metalloelastase), MMP-19 (RASI-1, occasionally referred to as stromelysin-4), Enamelysin (MMP-20), and MMP-27 (MMP-22, C-MMP), MMP-23A (CA-MMP), and MMP-23B.

MMPs can be used in combination with each other and with other biomarkers in the disclosed methods and as indicators of LVH and CHF. MMPs can be combined with each other and with any other biomarker or combination of biomarkers.

2. TIMPs

The MMPs are inhibited by specific endogenous tissue inhibitor of metalloproteinases (TIMPs), which comprise a family of four protease inhibitors: TIMP-1, TIMP-2, TIMP-3 and TIMP-4. Overall, all MMPs are inhibited by TIMPs once they are activated but the gelatinases (MMP-2 and MMP-9) can form complexes with TIMPs when the enzymes are in the latent form. The complex of latent MMP-2 (pro-MMP-2) with TIMP-2 serves to facilitate the activation of pro-MMP-2 at the cell surface by MT1-MMP (MMP-14), a membrane-anchored MMP.

TIMPs can be used in combination with each other and with other biomarkers in the disclosed methods and as indicators of LVH and CHF. TIMPs can be combined with each other and with any other biomarker or combination of biomarkers.

3. Collagen Peptides

i. Propeptide for Collagen I (PINP)

Collagen is a family of fibrous proteins that are the major components of the extracellular matrix. It is the most abundant protein in mammals, constituting nearly 25% of the total protein in the body. Collagen plays a major structural role in the formation of bone, tendon, skin, cornea, cartilage, blood vessels, and teeth (Stryer, Biochemistry. 1988. W.H. Freeman, New York). The fibrillar types of collagen I, II, III, IV, V, and XI are all synthesized as larger trimeric precursors, called procollagens, in which the central uninterrupted triple-helical domain consisting of hundreds of “G-X-Y” repeats (or glycine repeats) is flanked by non-collagenous domains (NC), the N-propeptide and the C-propeptide (Stryer, Biochemistry. 1988. W.H. Freeman, New York). Both the C- and N-terminal extensions are processed proteolytically upon secretion of the procollagen, an event that triggers the assembly of the mature protein into collagen fibrils which forms an insoluble cell matrix (Prockop et al. J Struct Biol. 1998, 122, 111-118. Review).

Type I, IV, V and XI collagens are mainly assembled into heterotrimeric forms consisting of either two α-1 chains and one α-2 chain (for Type I, IV, V), or three different a chains (for Type XI), which are highly homologous in sequence.

The type II and III collagens are both homotrimers of α-1 chain. For type I collagen, the most abundant form of collagen, stable α-1 (1) homotrimer is also formed and is present at variable levels (Alvares et al., Biochemistry. 1999, 38, 5401-5411) in different tissues.

Reagents for detecting PINP are available from IDS Ltd. Kits include UniQ™ PINP RIA (Intact N-terminal Propeptide of Type I Procollagen) (OD-67034).

PINP can be used in combination with other biomarkers in the disclosed methods and as indicators of LVH and CHF. PINP can be combined with any other biomarker or combination of biomarkers.

ii. Propeptide for Collagen III (PIIINP)

Collagen III gene encodes a fibrillar collagen that is found in extensible connective tissues such as skin, lung, and the vascular system, and is also frequently in association with type I collagen. Propeptide for collagen III (PIIINP) occurs as a trimer consisting of three identical monomeric PIIINP subunits that are linked by intermolecular disulfide bridges. PIIINP in turn is structurally divided into three domains. Structural considerations and site-directed mutagenesis experiments with a collagen mini-gene (Lees and Bulleid, Biol. Chem. 1994, 269, 24354-24360) have led to the conclusion that at least 4 and probably 6 cystine residues are involved in intramolecular disulfide bridge formation and that only cystines 51 and 68 are involved in intermolecular disulfide bridge formation. It has been observed, however, that the region around this intermolecular cystine bridge is critical for the correct formation of intramolecular disulfide bridges and that a Cys to Ser mutation in that region also leads to impaired intramolecular disulfide bridge formation. On the other hand it has been observe that the trimerization of collagen (III) fibrils can proceed even when interchain cystine bridge formation has become impossible by Cys to Ser mutations in position CysS I (relative to the C-proteinase cleavage site) (Lees and Bulleid, Biol. Chem. 1994, 269, 24354-24360). The most N-terminally located domain (Col1) consists of a globular structure linked by several intramolecular cystine bridges. Col3 is the intermediate domain and possesses a collagen-like structure characterized by periodic Gly and Pro residues. This domain assembles into a characteristic triple-helical collagen-like structure characterizing the Col3 domain. The Col2 domain encompasses the parts of the procollagen telopeptide region proximal to the N-proteinase cleavage site. The three monomeric PIIINP subunits are assembled as parallel peptide strands in this domain. Characteristically, the Col2 domain contains two cystein residues that are both involved in intermolecular disulfide bridge formation and that are solely responsible for the trimeric structure of PIIINP (Kuhn et al., Connect Tissue Res. 1982, 10, 5-10). Furthermore, The N-terminal procollagen (III) propeptide (PIIINP) molecule is a proteolytic fragment emanating from the specific cleavage of procollagen (III) by N-proteinase after exocytosis.

Trimeric PIIINP emanating from collagen synthesis and cleaved by N-protease is usually the most abundant PIIINP species but its relative proportion is not constant (Niemela et at, Clin Chim Act. 1982, 1, 39-44).

It has been firmly established that the nucleation of triple helix formation in collagens I and III starts from the C-terminal region of the procollagen molecules and proceeds in a more or less “zipper-like” fashion towards the N-terminus (Engel and Prockop, Annu Rev Biophys Biophys Chem. 1991, 20, 137-152). The molecular interactions governing the association of PIIIP to form the al (III) homotrimer are so far poorly understood.

From molecular sieve experiments with patient sera it has been shown that serum assays recognizing PIIINP detect three different molecular weight forms. The fraction containing lower molecular weight species consists of monomeric Col1 domains. The absolute amount of this circulating Col1 domain is relatively constant in healthy volunteers as well as in patients with chronic active hepatitis and acute alcoholic hepatitis. In addition to the circulating Col1 domain fragment, the antibodies also recognize higher than trimeric PIIINP species in the sera. The exact molecular nature of these high molecular weight species is not known.

A number of patents have dealt with the problem of PIIINP determinations from patient sera and with methods to improve the diagnostic validity of these determinations. For the measurement of PIIINP in sera of patients with liver diseases several different PIIINP radioimmunoassays have been reported. EP 0004940A1 by Timpl et al., 1979, describes a non-equilibrium inhibition radioimmunoassay based on a bovine antigen-antibody system which shows cross-reactivity with human PIIINP.

Reagents for detecting PIIINP are available from IDS Ltd. Kits include UniQ™ PIIINP RIA (Intact N-terminal Propeptide of Type III Procollagen) (OD-06098).

PIIINP can be used in combination with other biomarkers in the disclosed methods and as indicators of LVH and CHF. PIIINP can be combined with any other biomarker or combination of biomarkers.

iii. C-Telopeptide for Type-I Collagen (CITP)

Secreted trimeric C-propeptide of type I collagen is found in the blood of normal people at a concentration in the range of 100-600 ng/mL, with children having a higher level which is indicative with active bone formation. Most of these collagen C-propeptide chains can self-assemble into homotrimers, when over-expressed alone in a cell. Although the N-propeptide domains are synthesized first, molecular assembly into trimeric collagen begins with the in-register association of the C-propeptides.

Reagents for detecting CITP are available from IDS Ltd. Kits include UniQ™ ICTP EIA (C-terminal Telopeptide of Type I Collagen) (OD-06096)

CITP can be used in combination with other biomarkers in the disclosed methods and as indicators of LVH and CHF. CITP can be combined with any other biomarker or combination of biomarkers.

4. Soluble Receptor for Advanced Glycated End Products (sRAGE)

Incubation of proteins or lipids with aldose sugars results in nonenzymatic glycation and oxidation of amino groups on proteins to form Amadori adducts. Over time, the adducts undergo additional rearrangements, dehydrations, and cross-linking with other proteins to form complexes known as Advanced Glycosylation End Products (AGEs). Factors which promote formation of AGEs included delayed turnover (e.g. as in amyloidoses), accumulation of macromolecules having high lysine content protein and high blood glucose levels (e.g. as in diabetes) (Hori et al., J. Biol. Chem. 270: 25752-761, (1995)). AGEs have implicated in a variety of disorders including complications associated with diabetes and normal aging. AGEs display specific and saturable binding to cell surface receptors on endothelial cells of the microvasculature, monocytes and macrophages, smooth muscle cells, mesengial cells, and neurons. The Receptor for Advanced Glycated Endproducts (RAGE) is a member of the immunoglobulin super family of cell surface molecules. The extracellular (N-terminal) domain of RAGE includes three immunoglobulin-type regions, one V (variable) type domain followed by two C-type (constant) domains (Neeper et al., J. Biol. Chem. 267: 14998-15004 (1992)). A single transmembrane spanning domain and a short, highly charged cytosolic tail follow the extracellular domain. The N-terminal, extracellular domain can be isolated by proteolysis of RAGE to generate soluble RAGE (sRAGE) comprised of the V and C domains. RAGE is expressed in most tissues, and in particular, is found in cortical neurons during embryogenesis (Hori et al., J. Biol. Chem. 270: 25752-761 (1995)). Increased levels of RAGE are also found in aging tissues (Schleicher et al., J. Clin. Invest. 99 (3): 457-468 (1997)), and the diabetic retina, vasculature and kidney (Schmidt et al., Nature Med. 1: 1002-1004 (1995)). Activation of RAGE in different tissues and organs leads to a number of pathophysiological consequences. RAGE has been implicated in a variety of conditions including: acute and chronic inflammation (Hofmann et al., Cell 97: 889-901 (1999)), the development of diabetic late complications such as increased vascular permeability (Wautier et al., J. Clin. Invest. 97: 238-243 (1995)), nephropathy.

Assays and reagents for measuring sRAGE are described in Falcone et al., “Plasma Levels of Soluble Receptor for Advanced Glycation End Products and Coronary Artery Disease in Nondiabetic Men,” Arteriosclerosis, Thrombosis, and Vascular Biology (2005) 25:1032; and Kalousová et al., “Receptor for advanced glycation end products—soluble form and gene polymorphisms in chronic haemodialysis patients,” Nephrology Dialysis Transplantation (2007) 1-7.

sRAGE can be used in combination with each other and with other biomarkers in the disclosed methods and as indicators of LVH and CHF. sRAGE can be combined with each other and with any other biomarker or combination of biomarkers.

5. Cardiotrophin

Cardiotrophin 1 (CT-1) is a 201 amino acid member of the interleukin-6 superfamily. It was identified by its ability to induce hypertrophic response in cardiac. It is a cardiac hypertrophic factor of 21.5 kDa and a protein member of the interleukin-6 cytokine and leukaemia inhibitory factor family (Wollert et al. J. Biol. Chem. 1996, 271, 9535-9545). The receptors for each of these factors contain a common expressed surface polypeptide known as gp130 (glycoprotein 130) and also a second receptor component known as leukaemia inhibitory factor receptor unit β (Pennica et al. J. Biol. Chem. 1995, 270, 10915). CT 1 activates gp130 dependent signaling and stimulates the Janus kinase/signal transducers and activators of transcription (JAK/STAT) pathway to transduce hypertrophic and cytoprotective signals in cardiac myocytes. CT-1 is associated with the pathophysiology of heart diseases, including hypertension, myocardial infarction, valvular heart disease, and congestive heart failure. In addition, CT-1 activates phosphatidylinositol 3-kinase (PI-3 kinase) in cardiac myocytes and enhances transcription factor NF-κB DNA-binding activities. The genes encoding both human and mouse CT-1 have been cloned (Pennica, D., et al., (1995) Cytokine, 8: 1.83-189, Pennica, I)., et al., (1995) Proc. Natl. Acad. Sci. USA, 92: 1142-1146). CT-1 is highly expressed in the heart, skeletal muscle, prostate and ovary and to lower levels in lung, kidney, pancreas, thymus, testis and small intestine. In the heart, CT-1 is primarily expressed in myocardial cells, and not in endocardial cushion or outflow tract tissues. CT-1 can play an autocrine role during cardiac chamber growth and morphogenesis by promoting the survival and proliferation of immature myocytes via a gp130-dependent signaling pathway (Sheng et al. Development, 1996, 122, 419-428). CT-1 has its use in the diagnosis and treatment of cancer (LJ. S. Patent. Application 20020146707). Protection of adult rat hearts from injury by administration of CT-1 prior to ischaemia has also been reported (Liao, Z., et al., (2002) Cardiovasc. Res., 53: 902-910).

Reagents for detecting cardiotrophin are available from R&D Systems. Kits include Biotinylated Anti-mouse Cardotrophpin-1 Antibody (BAF438).

Cardiotrophin can be used in combination with other biomarkers in the disclosed methods and as indicators of LVH and CHF. Cardiotrophin can be combined with any other biomarker or combination of biomarkers.

6. Osteopontin

Secreted phosphoprotein 1 (osteopontin, bone sialoprotein I, early T-lymphocyte activation 1), also known as SPP1 and commonly referred to as osteopontin, is a human gene (Du et al. J. Mol. Biol. 2008, 382, 835.) Osteopontin is a glycoprotein first identified in 1986 in osteoblasts. The prefix of the word “osteo” indicates that the protein is expressed in bone. The suffix “-pontin” is derived from “pons,” the Latin word for bridge, and signifies osteopontin's role as a linking protein. Osteopontin is an extracellular structural protein and therefore an organic component of bone. Synonyms for this protein include sialoprotein 1 and 44K BPP (bone phosphoprotein). The gene has 7 exons, spans 5 kilobases in length and is located on the long arm of human chromosome 4q. Osteopontin is a single-chain polypeptide with a molecular weight of approximately 32,600 (Oldberg et al. Proc Natl Acad Sci, 1986, 83, 8819-8823). The protein is composed of ˜300 amino acids residues and has ˜30 carbohydrate residues attached including 10 sialic acid residues. The carbohydrate residues are attached to the protein during its residence in the Golgi apparatus. The protein is rich in acidic residues: 30-36% are either aspartic or glutamic acid. Osteopontin is a secreted, extracellular matrix-associated protein and has diverse biological activities many of which make it of great interest for study in relation to insulin resistance, and type 2 diabetes (Wai, P. Y., et al. 2004. J Surg Res 121:228-24), Osteopontin binds to osteoclasts in vitro via the avb3 integrin (Flores et al., Exp Cell Res. 1992, 201,:526-30). In the bone it is much enriched at the site where osteoclasts are attached to the underlying mineral surface. i.e. in the clear zone attachment area of the plasma membrane (Reinholt et al. Proc Natl Acad. Sci. 1990 87, 4473-5). The ligand avb3 integrin of the osteoclast is also much enriched in the corresponding location on the clear zone region of the membrane (Reinholt et al. Proc Natl Acad. Sci. 1990 87, 4473-5). Addition of RGD-integrin binding peptides is able to interfere with bone turnover (Horton et al. Exp Cell Res. 1991, 195, 368-75) indicating a key role for osteopontin. OPN dephosphorylated with TRAP (tatrate-resistant acid phosphatase) secreted by the osteoclasts has been shown in vitro not to support binding of osteoclasts (Reinholt et al. Proc Natl Acad. Sci. 1990 87, 4473-5), indicating a potential release mechanism for these cells.

Reagents for detecting cardiotrophin are available from IBL-America. Kits include Human Osteopontin Assay Kit (L) (Code No. 27158).

Osteopontin can be used in combination with other biomarkers in the disclosed methods and as indicators of LVH and CHF. Osteopontin can be combined with any other biomarker or combination of biomarkers.

7. N-Terminal Pro-B-Type Natriuretic Peptide (NTBNP)

Human B-type Natriuretic Peptide (BNP), a member of the cardiac natriuretic peptide family, is a 32 amino acid peptide with potent natriuretic, diuretic and vasodilatatory endocrine functions. The BNP gene is predominantly expressed in the myocytes of the failing heart with BNP increasingly secreted into the circulation in patients with congestive heart failure. Consequently, the diagnostic use of plasma BNP measurements has been studied (Mair et al., Clin Chem Lab Med. 2001, 39, 571-588). Increased plasma concentrations of BNP are associated with impaired function of the left ventricle disregarding the underlying cause and are therefore valuable in the primary diagnosis of heart failure.

The BNP gene encodes preproBNP, a 134 amino acid residue precursor in which proBNP contains 108 amino acid residues and the bioactive BNP-32 sequence constitutes the C-terminus. In 1995, Hum et al., Biochem Biophys Res Commun. 1995, 214, 1175-1183, showed that a fragment N-terminal of the active peptide also circulates in plasma and that the concentration increases in heart failure patients. Chromatographic studies have at least indicated the presence of a high molecular weight proBNP peptide (known in the art as proBNP or BNP1 108) as well as a shorter N-terminal fragment, most likely to be a 1-76 fragment (known in the art as proBNP, 76), in plasma from patients with congestive heart failure, however, a complete understanding of the molecular heterogeneity of proBNP-derived peptides in plasma is yet to be realized (Hunt et al. Biochem Biophys Res Commun. 1995, 214, 1175-1183, and 1997a; Schultz et al., 2001).

Several assays directed against both the N-terminal portions of proBNP and bioactive BNP-32 have now been developed (Hunt et al., Biochem Biophys Res Commun. 1995, 214, 1175-1183, and 1997a, b; Schultz et al., 2001; Karl et al., 1999; Hughes et al., Clin Sci (Loud). 1999, 96, 373-380; Campbell et al., J Card Fail. 2000 6, 130-139; U.S. Pat. No. 6,124,430; U.S. Pat. No. 5,786,163) and generally, the plasma concentrations of these portions, like bioactive BNP-32, have been reported to be elevated in patients with heart failure.

Reagents for detecting CITP are available from Bio-Stat Diagnostic Systems (PATHFAST™-NTproBNP) and BioMérieux (VIDAS NT-proBNP).

NTBNP can be used in combination with other biomarkers in the disclosed methods and as indicators of LVH and CHF. NTBNP can be combined with any other biomarker or combination of biomarkers.

8. Ratios

One of the unique characteristics for the disclosed profiling in hypertensive heart disease is to utilize ratios of biomarkers as indicators of LVH, CHF, DHF and/or the risk of LVH, CHF, and DHF. For example, the cardiac specific TIMP, TIMP-4, can be used and placed in context with an MMP which changes in greater magnitude in myocardial infarction and hypertensive patients. Also disclosed are ratios of an MMP, such as MMP-9 or MMP-13, to a TIMP, such as TIMP-1, TIMP-2, or TIMP-4. Also disclosed are ratios of a peptide for collagen, such as PIIINP, to another peptide for collagen, such as PINP or CITP. These ratios are used herein as diagnostic differentials and for identifying patients with distinctly different disease states.

Ratios of biomarkers can be used in combination with each other and with other biomarkers in the disclosed methods and as indicators of LVH and CHF. Ratios of biomarkers can be combined with each other and with any other biomarker or combination of biomarkers.

9. Gender

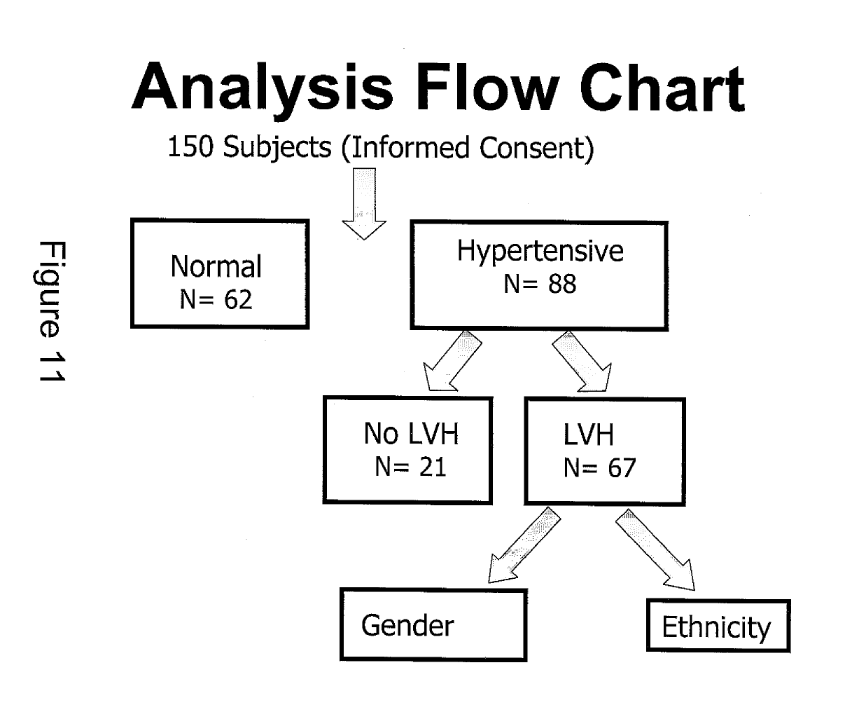

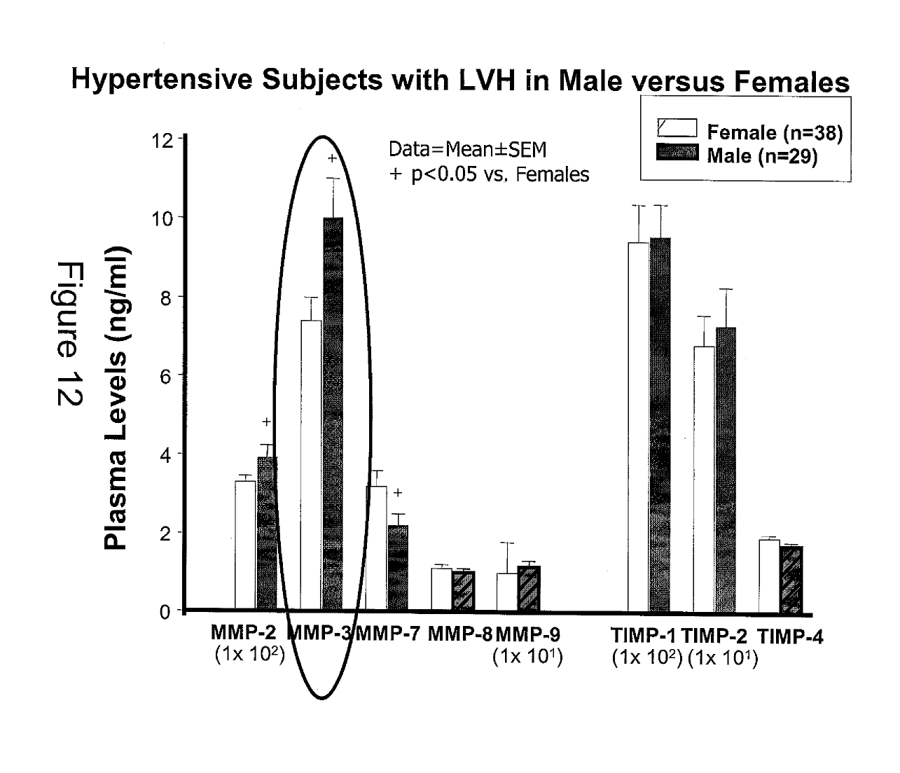

Gender can serve as an independent variable in the disclosed biomarker profiling. As examples, it has been discovered that gender influences MMP-7 levels independently with respect to LVH, and that MMP-2 levels are differentially regulated in males and females with LVH. Example data regarding gender as a biomarker is described in Example 6 and in FIGS. 11 and 12. Thus, the gender of the subject can be used in combination with other biomarkers in the disclosed methods and as indicators of LVH and CHF. Gender can be combined with any other biomarker or combination of biomarkers.

As an example, where the subject is male, an amount of MMP-3 that is greater than the reference amount (for example, the amount in a normal subject) can indicate the presence or risk of congestive heart failure in the subject. The amount of MMP-3 can be at least about 50% greater than the reference amount. As another example, where the subject is male, an amount of MMP-2 that is greater than the reference amount (for example, the amount in a normal subject) can indicate the presence or risk of congestive heart failure in the subject. The amount of MMP-2 can be at least about 10% greater than the reference amount.

As another example, where the subject is female, an amount of MMP-7 that is greater than the reference amount (for example, the amount in a normal subject) can indicate the presence or risk of congestive heart failure in the subject. The amount of MMP-7 can be at least about 30% greater than the reference amount. As another example, where the subject is female, an amount of MMP-8 that is greater than the reference amount (for example, the amount in a normal subject) can indicate the presence or risk of congestive heart failure in the subject. The amount of MMP-8 can be at least about 50% greater than the reference amount.

10. Ethnicity

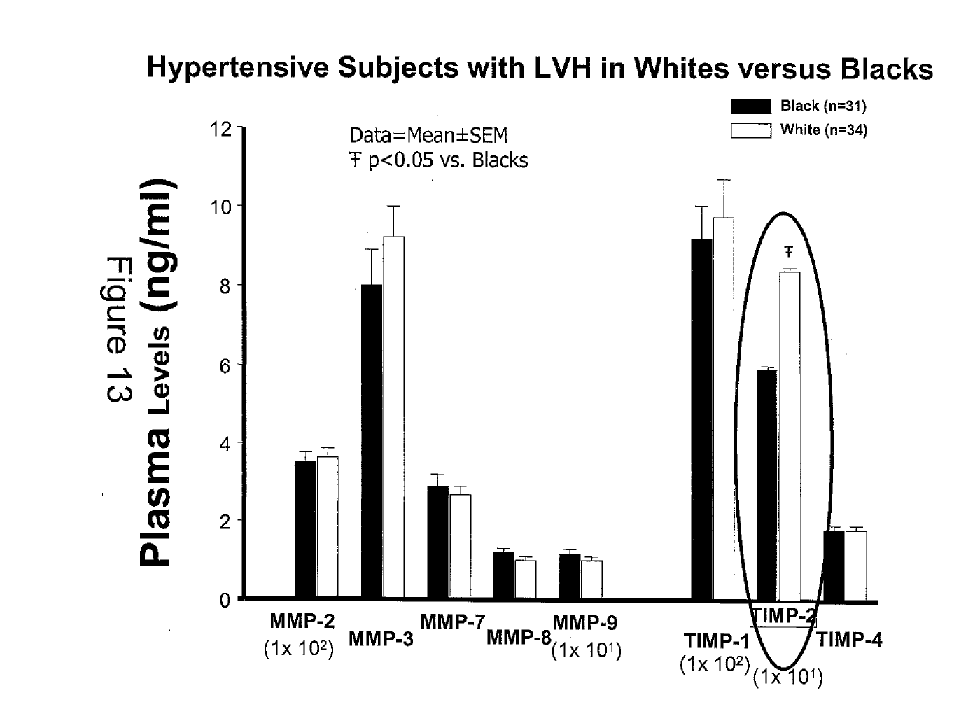

Ethnicity can serve as an independent variable in the disclosed biomarker profiling. As an example, it has been discovered that ethnicity influences TIMP-2 levels independently with respect to LVH. Example data regarding gender as a biomarker is described in Example 6 and in FIGS. 11 and 13. Thus, the ethnicity of the subject can be used in combination with other biomarkers in the disclosed methods and as indicators of LVH and CHF. Ethnicity can be combined with any other biomarker or combination of biomarkers.

As an example, where the subject is black, an amount of TIMP-2 that is less than the reference amount (for example, the amount in a normal subject, the amount in a subject with hypertension, or the amount in a subject with left ventricular hypertrophy) indicates the presence or risk of congestive heart failure in the subject. The amount of TIMP-2 can be at least about 10% less than the reference amount.

### B. Antibodies

Antibodies specific for MMPs and TIMPs and the other biomarkers are known and commercially available. Examples of antibodies are provided in Table 2.

TABLE 2
MMP/TIMP Antibodies

| Analyte | Assay | Catalog # | Vendor |
|---|---|---|---|
| MMP-1 | | IM52 | Oncogene |
| | | PC311 | Oncogene |
| | | IM35L | Oncogene |
| | | AB806 | Chemicon |
| | MSA | LMP901 | RnD Systems |
| MMP-2 | | AB19015 | Chemicon |
| | | PC342 | Oncogene |
| | | IM33L | Oncogene |
| | | MAB3308 | Chemicon |
| | | AB19015 | Chemicon |
| | | MAB13405 | Chemicon |
| | | AB809 | Chemicon |
| | MSA | LMP902 | RnD Systems |
| MMP-3 | | PC310 | Oncogene |
| | | AB810 | Chemicon |
| | | AB811 | Chemicon |
| | | IM36L | Oncogene |
| | MSA | LMP513 | RnD Systems |
| MMP-7 | | PC492 | Oncogene |
| | | AB8118 | Chemicon |
| | | AB8117 | Chemicon |
| | MSA | LMP907 | RnD Systems |
| MMP-8 | | 3528-100 | BioVision |
| | | PC493 | Oncogene |
| | | IM38L | Oncogene |
| | MSA | LMP908 | RnD Systems |
| MMP-9 | | AB19047 | Chemicon |
| | | IM09 | Oncogene |
| | | PC309 | Oncogene |
| | | AB804 | Chemicon |
| | MSA | LMP911 | RnD Systems |
| MMP-11 | | PC467 | Oncogene |
| MMP-12 | | AB19051 | Chemicon |
| | | RPI-MMP-12 | TriplePointBiologics |
| | | PC494 | Oncogene |
| | MSA | LMP919 | RnD Systems |
| MMP-13 | | AB8114 | Chemicon |
| | | PC542 | Oncogene |
| | | 3533-100 | BioVision |
| | | AB19055 | Chemicon |
| | MSA | LMP511 | RnD Systems |
| MMP-14 | | AB815 | Chemicon |
| | | AB8102 | Chemicon |
| | | RDI-MMP14 | Res. Diagnostics, Inc. |
| | | MAB3317 | Chemicon |
| | | AB8221 | Chemicon |
| | | AB8103 | Chemicon |
| MMP-15 | | AB850 | Chemicon |
| | | MAB3320 | Chemicon |
| | | AB855 | Chemicon |
| TIMP-1 | | OPA1-08512 | ABR |
| | | AB8122 | Chemicon |
| | | AB770 | Chemicon |
| | | AB8116 | Chemicon |
| | | PC500 | Oncogene |
| | MSA | LKT003 | RnD Systems |
| TIMP-2 | | AB801 | Chemicon |
| | | RP2T2 | Triple Point Biologics |
| | | IM11L | Oncogene |
| | | CL1T2 | CedarLane |
| | | MAB3310 | Chemicon |
| | | AB8107 | Chemicon |
| | MSA | LKT003 | RnD Systems |
| TIMP-3 | | CL2T3 | CedarLane |
| | | IM43L | Oncogene |
| | | H-TIMP-3 | Triple Point Biologics |
| | MSA | LKT003 | RnD Systems |
| TIMP-4 | | AB816 | Chemicon |
| | | MAB974 | R&D Systems |
| | | Ab19087 | Chemicon |
| | MSA | LKT003 | RnD Systems |
| PINP | RIA | 67034 | Orion Diagnostics |
| PIIINP | RIA | 06098 | Orion Diagnostics |
| CITP | EIA | 06099 | Orion Diagnostics |
| Osteopontin | ELISA | DOST00 | RnD Systems |
| Cardiotrophin | ELISA | HCT-1 | Biovendor |
| sRAGE | ELISA | SRG00 | RnD Systems |

MSA = Multiplex Suspension Array
RIA = Radio-immunoassay
EIA = Enzyme Immunosorbent Assay
ELISA = Enzyme-linked Immunosorbent Assay

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with MMPs, TIMPs, or other biomarkers. The antibodies can be tested for their desired activity using the in vitro assays described herein, or by analogous methods, after which their in vivo therapeutic and/or prophylactic activities are tested according to known clinical testing methods.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855 (1984)).

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent.

The monoclonal antibodies may also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

### Methods

### A. Heart Failure

Congestive heart failure (CHF), also called congestive cardiac failure (CCF) or just heart failure, is a condition that can result from any structural or functional cardiac disorder that impairs the ability of the heart to fill with or pump a sufficient amount of blood throughout the body. Thus, the disclosed method can be used to treat any form of heart failure.

Because not all patients have volume overload at the time of initial or subsequent evaluation, the term “heart failure” is preferred over the older term “congestive heart failure”. Causes and contributing factors to congestive heart failure include the following (with specific reference to left (L) or right (R) sides): Genetic family history of CHF, Ischemic heart disease/Myocardial infarction (coronary artery disease), Infection, Alcohol ingestion, Heartworms, Anemia, Thyrotoxicosis (hyperthyroidism), Arrhythmia, Hypertension (L), Coarctation of the aorta (L), Aortic stenosis/regurgitation (L), Mitral regurgitation (L), Pulmonary stenosis/Pulmonary hypertension/Pulmonary embolism all leading to cor pulmonale (R), and Mitral valve disease (L).

There are many different ways to categorize heart failure, including: the side of the heart involved, (left heart failure versus right heart failure), whether the abnormality is due to contraction or relaxation of the heart (systolic heart failure vs. diastolic heart failure), and whether the abnormality is due to low cardiac output or low systemic vascular resistance (low-output heart failure vs. high-output heart failure).

Congestive heart failure (CHF) is a constellation of signs and symptoms (i.e. shortness of breath, fluid accumulation) due to an underlying disorder in cardiac performance—notably left ventricular (LV) function. The causes of CHF can be diverse, but fall into 3 main categories: following a heart attack (myocardial infarction), with hypertensive heart disease, and with intrinsic muscle disease generically called cardiomyopathy. It has been difficult to identify the underlying causes of CHF such as that caused by hypertensive heart disease, and this is focus of the present methods. Specifically, hypertensive heart disease causes growth of the LV muscle—called hypertrophy. LV hypertrophy (LVH) in and of itself can cause defects in cardiac performance, but a blood test to identify LVH quickly and accurately has not been available previously. This application identifies a new and validated approach to identify patients with, or at risk of, LVH. If the LVH process continues, or is not adequately treated, then patients will develop signs and symptoms of CHF primarily due to diastolic heart failure (DHF). However it has been difficult up to the present time to identify patients that suffer from CHF that primarily have DHF, and it has not been possible to identify these patients with a simple and rapid blood test. This application identifies a new and validated approach to identify patients that not only have the presence of LVH, but also those that will be at risk for the development of DHF, and identification of those that have DHF. Thus, the disclosed methods provide a means to detect the presence or risk of LVH, predict those patients that will be at risk for development of CHF and/or DHF, and to identify those patients with CHF and/or DHF. Through the use of, for example, a small sample of bodily fluid, and for the example identified below, a blood sample, it will be possible to perform, 4 independent, but not necessarily exclusive, applications of this method: screening, prediction/prognosis, diagnosis, and treatment monitoring.

Thus, disclosed is a method to, for example, diagnose a subject with left ventricular hypertrophy (LVH, HCM or HOCM), at risk for left ventricular hypertrophy, at risk for congestive heart failure and/or diastolic heart failure, or with congestive heart failure and/or diastolic heart failure. For example, provided is a method of detecting LVH in a subject, comprising identifying a profile of matrix metalloproteinases (MMPs), tissue inhibitors of matrix metalloproteinases (TIMPs), and/or other biomarkers (such as propeptide for collagen I (PINP), propeptide for collagen III (PIIINP), C-telopeptide for type-I collagen (CITP), cardiotrophin, soluble receptor for advanced glycated end products (sRAGE), osteopontin, and N-terminal pro-B-type natriuretic peptide (NTBNP)) from a body fluid of the subject that is associated as described herein with the existence or risk of left ventricular hypertrophy (LVH), congestive heart failure (CHF) and/or diastolic heart failure (DHF). Also provided is a method of, for example, predicting congestive heart failure and/or diastolic heart failure in a subject, comprising identifying a profile of matrix metalloproteinases (MMPs), tissue inhibitors of matrix metalloproteinases (TIMPs), and/or other biomarkers (such as propeptide for collagen I (PINP), propeptide for collagen III (PIIINP), C-telopeptide for type-I collagen (CITP), cardiotrophin, soluble receptor for advanced glycated end products (sRAGE), osteopontin, and N-terminal pro-B-type natriuretic peptide (NTBNP)) from a body fluid of the subject that is associated as described herein with the likely development of congestive heart failure (CHF) and/or diastolic heart failure (DHF).

Also disclosed is a method to, for example, determine the presence or risk of congestive heart failure in a subject, comprising measuring the amount of two or more biomarkers in a body fluid from a subject, wherein the amount of two or more of the two or more biomarkers compared to a reference amount for the biomarker (for example, the amount in a normal subject, the amount in a subject with hypertension, or the amount in a subject with left ventricular hypertrophy) indicates the presence or risk of congestive heart failure in the subject.

Also disclosed is a method to, for example, determine the presence or risk of left ventricular hypertrophy in a subject, comprising measuring the amount of two or more biomarkers in a body fluid from a subject, wherein the amount of two or more of the two or more biomarkers compared to a reference amount for the biomarker (for example, the amount in a normal subject, the amount in a subject with hypertension, or the amount in a subject with left ventricular hypertrophy) indicates the presence or risk of congestive heart failure in the subject.

The two or more biomarkers can be propeptide for collagen I (PINP), propeptide for collagen III (PIIINP), C-telopeptide for type-I collagen (CITP), cardiotrophin, soluble receptor for advanced glycated end products (sRAGE), osteopontin, MMP-2, MMP-3, MMP-7, MMP-8, MMP-9, MMP-13, TIMP-1, TIMP-2, TIMP-4, N-terminal pro-B-type natriuretic peptide (NTBNP), gender, and ethnicity.

At least one of the two or more biomarkers can be PINP, PIIINP, CITP, cardiotrophin, sRAGE, osteopontin, gender, or ethnicity. At least one of the two or more biomarkers can be PINP, PIIINP, CITP, cardiotrophin, sRAGE, osteopontin, gender, or ethnicity. At least one of the two or more biomarkers can be PINP, PIIINP, CITP, cardiotrophin, sRAGE, osteopontin, gender, or ethnicity, and at least one other of the two or more biomarkers can be a MMP or TIMP.

An amount of PIIINP that is greater than the reference amount can indicate the presence or risk of congestive heart failure in the subject. An amount of osteopontin that is greater than the reference amount can indicate the presence or risk of congestive heart failure in the subject. An amount of cardiotrophin that is less than the reference amount can indicate the presence or risk of congestive heart failure in the subject. An amount of sRAGE that is less than the reference amount can indicate the presence or risk of congestive heart failure in the subject. An amount of CITP that is greater than the reference amount can indicate the presence or risk of congestive heart failure in the subject. An increase in the ratio of PIIINP to PINP compared to a reference ratio can indicate the presence or risk of congestive heart failure in the subject. An increase in the ratio of PIIINP to CITP compared to a reference ratio can indicate the presence or risk of congestive heart failure in the subject. An amount of MMP-2 that is greater than the reference amount can indicate the presence or risk of congestive heart failure in the subject. An amount of MMP-3 that is less than the reference amount can indicate the presence or risk of congestive heart failure in the subject. An amount of MMP-7 that is greater than the reference amount can indicate the presence or risk of congestive heart failure in the subject. An amount of MMP-8 that is greater than the reference amount can indicate the presence or risk of congestive heart failure in the subject. An amount of MMP-9 that is greater than the reference amount can indicate the presence or risk of congestive heart failure in the subject. An amount of less than 10 ng/mL of MMP-13 can indicate the presence or risk of congestive heart failure in the subject. An amount of TIMP-1 that is greater than the reference amount can indicate the presence or risk of congestive heart failure in the subject. An amount of TIMP-2 that is less than the reference amount can indicate the presence or risk of congestive heart failure in the subject. An amount of TIMP-4 that is greater than the reference amount can indicate the presence or risk of congestive heart failure in the subject. A reduction in the ratio of MMP-9 to TIMP-1 compared to a reference ratio can indicate the presence or risk of congestive heart failure in the subject. A reduction in the ratio of MMP-9 to TIMP-2 compared to a reference ratio can indicate the presence or risk of congestive heart failure in the subject. A reduction in the ratio of MMP-9 to TIMP-4 compared to a reference ratio can indicate the presence or risk of congestive heart failure in the subject. An amount of NTBNP that is greater than the reference amount can indicate the presence or risk of congestive heart failure in the subject.

The amount of PIIINP in a body fluid from the subject can be measured and an amount of PIIINP that is greater than the reference amount indicates the presence or risk of congestive heart failure in the subject. The amount of PIIINP can be at least about 10% greater than the reference amount. The amount of osteopontin in a body fluid from the subject can be measured and an amount of osteopontin that is greater than the reference amount indicates the presence or risk of congestive heart failure in the subject. The amount of osteopontin can be at least about 20% greater than the reference amount. The amount of cardiotrophin in a body fluid from the subject can be measured and an amount of cardiotrophin that is less than the reference amount indicates the presence or risk of congestive heart failure in the subject. The amount of cardiotrophin can be at least about 30% less than the reference amount. The amount of sRAGE in a body fluid from the subject can be measured and an amount of sRAGE that is less than the reference amount indicates the presence or risk of congestive heart failure in the subject. The amount of sRAGE can be at least about 10% less than the reference amount. The amount of MMP-2 in a body fluid from the subject can be measured and an amount of MMP-2 that is greater than the reference amount indicates the presence or risk of congestive heart failure in the subject. The amount of MMP-2 can be at least about 10% greater than the reference amount. The amount of MMP-2 in a body fluid from the subject can be measured and an amount of MMP-2 that is greater than the reference amount indicates the presence or risk of congestive heart failure in the subject. The amount of MMP-2 can be at least about 10% greater than the reference amount. The amount of MMP-7 in a body fluid from the subject can be measured and an amount of MMP-7 that is greater than the reference amount indicates the presence or risk of congestive heart failure in the subject. The amount of MMP-7 can be at least about 30% greater than the reference amount. The amount of TIMP-2 in a body fluid from the subject can be measured and an amount of TIMP-2 that is less than the reference amount indicates the presence or risk of congestive heart failure in the subject. The amount of TIMP-2 can be at least about 10% less than the reference amount. The amount of TIMP-2 in a body fluid from the subject can be measured and an amount of TIMP-2 that is less than the reference amount indicates the presence or risk of congestive heart failure in the subject. The amount of TIMP-2 can be at least about 10% less than the reference amount. The amount of CITP in a body fluid from the subject can be measured and an amount of CITP that is greater than the reference amount indicates the presence or risk of congestive heart failure in the subject. The amount of CITP can be at least about 20% greater than the reference amount. The amount of PIIINP and CITP in a body fluid from the subject can be measured and an increase in the ratio of PIIINP to CITP compared to a reference ratio indicates the presence or risk of congestive heart failure in the subject, wherein the reference ratio is the ratio in a normal subject. The reduction in the ratio of PIIINP to CITP can be at least about 10% compared to the reference ratio. The amount of PIIINP and PINP in a body fluid from the subject can be measured and an increase in the ratio of PIIINP to PINP compared to a reference ratio indicates the presence or risk of congestive heart failure in the subject, wherein the reference ratio is the ratio in a normal subject. The reduction in the ratio of PIIINP to PINP can be at least about 20% compared to the reference ratio.

The amount of MMP-13 in a body fluid from the subject can be measured and an amount of less than 10 ng/mL of MMP-13 indicates the presence or risk of congestive heart failure in the subject. The amount of TIMP-1 in a body fluid from the subject can be measured and an amount of TIMP-1 that is greater than the reference amount indicates the presence or risk of congestive heart failure in the subject. The amount of TIMP-1 can be at least about 20% greater than the reference amount. The amount of TIMP-4 in a body fluid from the subject can be measured and an amount of TIMP-4 that is greater than the reference amount indicates the presence or risk of congestive heart failure in the subject. The amount of TIMP-4 can be at least about 50% greater than the reference amount. The amount of MMP-8 in a body fluid from the subject can be measured and an amount of MMP-8 that is greater than the reference amount indicates the presence or risk of congestive heart failure in the subject. The amount of MMP-8 can be at least about 50% greater than the reference amount. The amount of MMP-9 in a body fluid from the subject can be measured and an amount of MMP-9 that is greater than the reference amount indicates the presence or risk of congestive heart failure in the subject. The amount of MMP-9 can be at least about 50% greater than the reference amount. The amount of MMP-9 and TIMP-1 in a body fluid from the subject can be measured and a reduction in the ratio of MMP-9 to TIMP-1 compared to a reference ratio indicates the presence or risk of congestive heart failure in the subject, wherein the reference ratio is the ratio in a normal subject. The reduction in the ratio of MMP-9 to TIMP-1 can be at least about 50% compared to the reference ratio. The amount of MMP-9 and TIMP-2 in a body fluid from the subject can be measured and a reduction in the ratio of MMP-9 to TIMP-2 compared to a reference ratio indicates the presence or risk of congestive heart failure in the subject, wherein the reference ratio is the ratio in a normal subject. The reduction in the ratio of MMP-9 to TIMP-2 can be at least about 50% compared to the reference ratio. The amount of MMP-9 and TIMP-4 in a body fluid from the subject can be measured and a reduction in the ratio of MMP-9 to TIMP-4 compared to a reference ratio indicates the presence or risk of congestive heart failure in the subject, wherein the reference ratio is the ratio in a normal subject. The reduction in the ratio of MMP-9 to TIMP-4 can be at least about 50% compared to the reference ratio. The amount of MMP-3 in a body fluid from the subject can be measured and an amount of MMP-3 that is greater than the reference amount indicates the presence or risk of congestive heart failure in the subject. The amount of MMP-3 can be at least about 50% greater than the reference amount. The amount of NTBNP in a body fluid from the subject can be measured and an amount of NTBNP that is greater than the reference amount indicates the presence or risk of congestive heart failure in the subject. The amount of NTBNP can be at least about 50% greater than the reference amount.

### B. Plasma Screening

The disclosed methods afford a more rapid and simplified process to identify from a tissue or bodily fluid a subject at risk for developing adverse LVH as well as identify patients in which this process is occurring at an accelerated pace. Thus, the disclosed methods can comprise the detection of biomarkers in bodily fluid of the subject, such as blood, urine, plasma, serum, tears, lymph, bile, cerebrospinal fluid, interstitial fluid, aqueous or vitreous humor, colostrum, sputum, amniotic fluid, saliva, anal and vaginal secretions, perspiration, semen, transudate, exudate, and synovial fluid.

Blood plasma is the liquid component of blood, in which the blood cells are suspended. Plasma is the largest single component of blood, making up about 55% of total blood volume. Serum refers to blood plasma in which clotting factors (such as fibrin) have been removed. Blood plasma contains many vital proteins including fibrinogen, globulins and human serum albumin Sometimes blood plasma can contain viral impurities which must be extracted through viral processing.

### C. Immunoassay

There are numerous methods for detecting analytes, such as proteins, such as MMPs, TIMPs, and other biomarkers, known or newly discovered in the art, which can be used in the disclosed methods. For example, MMPs, TIMPs, and other biomarkers can be detected using standard immunodetection methods. The steps of various useful immunodetection methods have been described in the scientific literature, such as, e.g., Maggio et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986), each of which is incorporated herein by reference in its entirety and specifically for its teaching regarding immunodetection methods. Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

In general, immunoassays involve contacting a sample suspected of containing a molecule of interest (such as the disclosed biomarkers) with an antibody to the molecule of interest or contacting an antibody to a molecule of interest (such as antibodies to the disclosed biomarkers) with a molecule that can be bound by the antibody, as the case may be, under conditions effective to allow the formation of immunocomplexes. Contacting a sample with the antibody to the molecule of interest or with the molecule that can be bound by an antibody to the molecule of interest under conditions effective and for a period of time sufficient to allow the formation of immune complexes (primary immune complexes) is generally a matter of simply bringing into contact the molecule or antibody and the sample and incubating the mixture for a period of time long enough for the antibodies to form immune complexes with, i.e., to bind to, any molecules (e.g., antigens) present to which the antibodies can bind. In many forms of immunoassay, the sample-antibody composition, such as a tissue section, ELISA plate, dot blot or Western blot, can then be washed to remove any non-specifically bound antibody species, allowing only those antibodies specifically bound within the primary immune complexes to be detected.

Immunoassays can include methods for detecting or quantifying the amount of a molecule of interest (such as the disclosed biomarkers or their antibodies) in a sample, which methods generally involve the detection or quantitation of any immune complexes formed during the binding process. In general, the detection of immunocomplex formation is well known in the art and can be achieved through the application of numerous approaches. These methods are generally based upon the detection of a label or marker, such as any radioactive, fluorescent, biological or enzymatic tags or any other known label. See, for example, U.S. Pat. Nos. 3,817,837; 3,850,752; 3,939,350; 3,996,345; 4,277,437; 4,275,149 and 4,366,241, each of which is incorporated herein by reference in its entirety and specifically for teachings regarding immunodetection methods and labels.

As used herein, a label can include a fluorescent dye, a member of a binding pair, such as biotin/streptavidin, a metal (e.g., gold), or an epitope tag that can specifically interact with a molecule that can be detected, such as by producing a colored substrate or fluorescence. Substances suitable for detectably labeling proteins include fluorescent dyes (also known herein as fluorochromes and fluorophores) and enzymes that react with colorometric substrates (e.g., horseradish peroxidase). The use of fluorescent dyes is generally preferred in the practice of the disclosed methods as they can be detected at very low amounts. Furthermore, in the case where multiple antigens are reacted with a single array, each antigen can be labeled with a distinct fluorescent compound for simultaneous detection. Labeled spots on the array are detected using a fluorimeter, the presence of a signal indicating an antigen bound to a specific antibody.

Fluorophores are compounds or molecules that luminesce. Typically fluorophores absorb electromagnetic energy at one wavelength and emit electromagnetic energy at a second wavelength. Representative fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs-AutoFluorescent Protein-(Quantum Biotechnologies) see sgGFP, sgBFP; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO-TAGT™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; BOBO™-1; BOBO™−3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson-; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM I Methylcoumarin; CTC; CTC Formazan; Cy2™; Cy3.1 8; Cy3.5™; Cy3™; Cy5.1 8; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydrorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (DilC18(5)); DIDS; Dihydrorhodamine 123 (DHR); Dil (DilC18(3)); 1Dinitrophenol; DiO (DiOC18(3)); DiR; DiR (DiC18(7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; Fluor X; FM 1-43™; FM 4-46; Fura Red™ (high pH); Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer; (CCF2); GFP (S65T); GFP red shifted (rsGFP); GFP wild type' non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-lndo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin E8G; Oregon Green™; Oregon Green™ 488; Oregon Green™ 500; Oregon Green™514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-I PRO-3; Primuline; Procion Yellow; Propidium lodid (P1); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO3; YOYO-1; YOYO-3; Sybr Green; Thiazole orange (interchelating dyes); semiconductor nanoparticles such as quantum dots; or caged fluorophore (which can be activated with light or other electromagnetic energy source), or a combination thereof.

Labeling can be either direct or indirect. In direct labeling, the detecting antibody (the antibody for the molecule of interest) or detecting molecule (the molecule that can be bound by an antibody to the molecule of interest) include a label. Detection of the label indicates the presence of the detecting antibody or detecting molecule, which in turn indicates the presence of the molecule of interest or of an antibody to the molecule of interest, respectively. In indirect labeling, an additional molecule or moiety is brought into contact with, or generated at the site of, the immunocomplex. For example, a signal-generating molecule or moiety such as an enzyme can be attached to or associated with the detecting antibody or detecting molecule. The signal-generating molecule can then generate a detectable signal at the site of the immunocomplex. For example, an enzyme, when supplied with suitable substrate, can produce a visible or detectable product at the site of the immunocomplex. ELISAs use this type of indirect labeling.

As another example of indirect labeling, an additional molecule (which can be referred to as a binding agent) that can bind to either the molecule of interest or to the antibody (primary antibody) to the molecule of interest, such as a second antibody to the primary antibody, can be contacted with the immunocomplex. The additional molecule can have a label or signal-generating molecule or moiety. The additional molecule can be an antibody, which can thus be termed a secondary antibody. Binding of a secondary antibody to the primary antibody can form a so-called sandwich with the first (or primary) antibody and the molecule of interest. The immune complexes can be contacted with the labeled, secondary antibody under conditions effective and for a period of time sufficient to allow the formation of secondary immune complexes. The secondary immune complexes can then be generally washed to remove any non-specifically bound labeled secondary antibodies, and the remaining label in the secondary immune complexes can then be detected. The additional molecule can also be or include one of a pair of molecules or moieties that can bind to each other, such as the biotin/avadin pair. In this mode, the detecting antibody or detecting molecule should include the other member of the pair.

Other modes of indirect labeling include the detection of primary immune complexes by a two step approach. For example, a molecule (which can be referred to as a first binding agent), such as an antibody, that has binding affinity for the molecule of interest or corresponding antibody can be used to form secondary immune complexes, as described above. After washing, the secondary immune complexes can be contacted with another molecule (which can be referred to as a second binding agent) that has binding affinity for the first binding agent, again under conditions effective and for a period of time sufficient to allow the formation of immune complexes (thus forming tertiary immune complexes). The second binding agent can be linked to a detectable label or signal-generating molecule or moiety, allowing detection of the tertiary immune complexes thus formed. This system can provide for signal amplification.

Immunoassays that involve the detection of as substance, such as a protein or an antibody to a specific protein, include label-free assays, protein separation methods (i.e., electrophoresis), solid support capture assays, or in vivo detection. Label-free assays are generally diagnostic means of determining the presence or absence of a specific protein, or an antibody to a specific protein, in a sample. Protein separation methods are additionally useful for evaluating physical properties of the protein, such as size or net charge. Capture assays are generally more useful for quantitatively evaluating the concentration of a specific protein, or antibody to a specific protein, in a sample. Finally, in vivo detection is useful for evaluating the spatial expression patterns of the substance, i.e., where the substance can be found in a subject, tissue or cell.

Provided that the concentrations are sufficient, the molecular complexes ([Ab-Ag]n) generated by antibody-antigen interaction are visible to the naked eye, but smaller amounts may also be detected and measured due to their ability to scatter a beam of light. The formation of complexes indicates that both reactants are present, and in immunoprecipitation assays a constant concentration of a reagent antibody is used to measure specific antigen ([Ab-Ag]n), and reagent antigens are used to detect specific antibody ([Ab-Ag]n). If the reagent species is previously coated onto cells (as in hemagglutination assay) or very small particles (as in latex agglutination assay), “clumping” of the coated particles is visible at much lower concentrations. A variety of assays based on these elementary principles are in common use, including Ouchterlony immunodiffusion assay, rocket immunoelectrophoresis, and immunoturbidometric and nephelometric assays. The main limitations of such assays are restricted sensitivity (lower detection limits) in comparison to assays employing labels and, in some cases, the fact that very high concentrations of analyte can actually inhibit complex formation, necessitating safeguards that make the procedures more complex. Some of these Group 1 assays date right back to the discovery of antibodies and none of them have an actual “label” (e.g. Ag-enz). Other kinds of immunoassays that are label free depend on immunosensors, and a variety of instruments that can directly detect antibody-antigen interactions are now commercially available. Most depend on generating an evanescent wave on a sensor surface with immobilized ligand, which allows continuous monitoring of binding to the ligand. Immunosensors allow the easy investigation of kinetic interactions and, with the advent of lower-cost specialized instruments, may in the future find wide application in immunoanalysis.

The use of immunoassays to detect a specific protein can involve the separation of the proteins by electophoresis. Electrophoresis is the migration of charged molecules in solution in response to an electric field. Their rate of migration depends on the strength of the field; on the net charge, size and shape of the molecules and also on the ionic strength, viscosity and temperature of the medium in which the molecules are moving. As an analytical tool, electrophoresis is simple, rapid and highly sensitive. It is used analytically to study the properties of a single charged species, and as a separation technique.

Generally the sample is run in a support matrix such as paper, cellulose acetate, starch gel, agarose or polyacrylamide gel. The matrix inhibits convective mixing caused by heating and provides a record of the electrophoretic run: at the end of the run, the matrix can be stained and used for scanning, autoradiography or storage. In addition, the most commonly used support matrices-agarose and polyacrylamide-provide a means of separating molecules by size, in that they are porous gels. A porous gel may act as a sieve by retarding, or in some cases completely obstructing, the movement of large macromolecules while allowing smaller molecules to migrate freely. Because dilute agarose gels are generally more rigid and easy to handle than polyacrylamide of the same concentration, agarose is used to separate larger macromolecules such as nucleic acids, large proteins and protein complexes. Polyacrylamide, which is easy to handle and to make at higher concentrations, is used to separate most proteins and small oligonucleotides that require a small gel pore size for retardation.

Proteins are amphoteric compounds; their net charge therefore is determined by the pH of the medium in which they are suspended. In a solution with a pH above its isoelectric point, a protein has a net negative charge and migrates towards the anode in an electrical field. Below its isoelectric point, the protein is positively charged and migrates towards the cathode. The net charge carried by a protein is in addition independent of its size—i.e., the charge carried per unit mass (or length, given proteins and nucleic acids are linear macromolecules) of molecule differs from protein to protein. At a given pH therefore, and under non-denaturing conditions, the electrophoretic separation of proteins is determined by both size and charge of the molecules.

Sodium dodecyl sulphate (SDS) is an anionic detergent which denatures proteins by “wrapping around” the polypeptide backbone—and SDS binds to proteins fairly specifically in a mass ratio of 1.4:1. In so doing, SDS confers a negative charge to the polypeptide in proportion to its length. Further, it is usually necessary to reduce disulphide bridges in proteins (denature) before they adopt the random-coil configuration necessary for separation by size; this is done with 2-mercaptoethanol or dithiothreitol (DTT). In denaturing SDS-PAGE separations therefore, migration is determined not by intrinsic electrical charge of the polypeptide, but by molecular weight.

Determination of molecular weight is done by SDS-PAGE of proteins of known molecular weight along with the protein to be characterized. A linear relationship exists between the logarithm of the molecular weight of an SDS-denatured polypeptide, or native nucleic acid, and its Rf. The Rf is calculated as the ratio of the distance migrated by the molecule to that migrated by a marker dye-front. A simple way of determining relative molecular weight by electrophoresis (Mr) is to plot a standard curve of distance migrated vs. log 10MW for known samples, and read off the log Mr of the sample after measuring distance migrated on the same gel.

In two-dimensional electrophoresis, proteins are fractionated first on the basis of one physical property, and, in a second step, on the basis of another. For example, isoelectric focusing can be used for the first dimension, conveniently carried out in a tube gel, and SDS electrophoresis in a slab gel can be used for the second dimension. One example of a procedure is that of O'Farrell, P. H., High Resolution Two-dimensional Electrophoresis of Proteins, J. Biol. Chem. 250:4007-4021 (1975), herein incorporated by reference in its entirety for its teaching regarding two-dimensional electrophoresis methods. Other examples include but are not limited to, those found in Anderson, L and Anderson, N G, High resolution two-dimensional electrophoresis of human plasma proteins, Proc. Natl. Acad. Sci. 74:5421-5425 (1977), Ornstein, L., Disc electrophoresis, L. Ann N.Y. Acad. Sci. 121:321349 (1964), each of which is herein incorporated by reference in its entirety for teachings regarding electrophoresis methods.

Laemmli, U.K., Cleavage of structural proteins during the assembly of the head of bacteriophage T4, Nature 227:680 (1970), which is herein incorporated by reference in its entirety for teachings regarding electrophoresis methods, discloses a discontinuous system for resolving proteins denatured with SDS. The leading ion in the Laemmli buffer system is chloride, and the trailing ion is glycine. Accordingly, the resolving gel and the stacking gel are made up in Tris-HCl buffers (of different concentration and pH), while the tank buffer is Tris-glycine. All buffers contain 0.1% SDS.

One example of an immunoassay that uses electrophoresis that is contemplated in the current methods is Western blot analysis. Western blotting or immunoblotting allows the determination of the molecular mass of a protein and the measurement of relative amounts of the protein present in different samples. Detection methods include chemiluminescence and chromagenic detection. Standard methods for Western blot analysis can be found in, for example, D. M. Bollag et al., Protein Methods (2d edition 1996) and E. Harlow & D. Lane, Antibodies, a Laboratory Manual (1988), U.S. Pat. No. 4,452,901, each of which is herein incorporated by reference in their entirety for teachings regarding Western blot methods. Generally, proteins are separated by gel electrophoresis, usually SDS-PAGE. The proteins are transferred to a sheet of special blotting paper, e.g., nitrocellulose, though other types of paper, or membranes, can be used. The proteins retain the same pattern of separation they had on the gel. The blot is incubated with a generic protein (such as milk proteins) to bind to any remaining sticky places on the nitrocellulose. An antibody is then added to the solution which is able to bind to its specific protein.

The attachment of specific antibodies to specific immobilized antigens can be readily visualized by indirect enzyme immunoassay techniques, usually using a chromogenic substrate (e.g. alkaline phosphatase or horseradish peroxidase) or chemiluminescent substrates. Other possibilities for probing include the use of fluorescent or radioisotope labels (e.g., fluorescein, ¹²⁵I). Probes for the detection of antibody binding can be conjugated anti-immunoglobulins, conjugated staphylococcal Protein A (binds IgG), or probes to biotinylated primary antibodies (e.g., conjugated avidin/streptavidin).

The power of the technique lies in the simultaneous detection of a specific protein by means of its antigenicity, and its molecular mass. Proteins are first separated by mass in the SDS-PAGE, then specifically detected in the immunoassay step. Thus, protein standards (ladders) can be run simultaneously in order to approximate molecular mass of the protein of interest in a heterogeneous sample.

The gel shift assay or electrophoretic mobility shift assay (EMSA) can be used to detect the interactions between DNA binding proteins and their cognate DNA recognition sequences, in both a qualitative and quantitative manner. Exemplary techniques are described in Ornstein L., Disc electrophoresis-I: Background and theory, Ann. NY Acad. Sci. 121:321-349 (1964), and Matsudiara, P T and D R Burgess, SDS microslab linear gradient polyacrylamide gel electrophoresis, Anal. Biochem. 87:386-396 (1987), each of which is herein incorporated by reference in its entirety for teachings regarding gel-shift assays.

In a general gel-shift assay, purified proteins or crude cell extracts can be incubated with a labeled (e.g., ³²P-radiolabeled) DNA or RNA probe, followed by separation of the complexes from the free probe through a nondenaturing polyacrylamide gel. The complexes migrate more slowly through the gel than unbound probe. Depending on the activity of the binding protein, a labeled probe can be either double-stranded or single-stranded. For the detection of DNA binding proteins such as transcription factors, either purified or partially purified proteins, or nuclear cell extracts can be used. For detection of RNA binding proteins, either purified or partially purified proteins, or nuclear or cytoplasmic cell extracts can be used. The specificity of the DNA or RNA binding protein for the putative binding site is established by competition experiments using DNA or RNA fragments or oligonucleotides containing a binding site for the protein of interest, or other unrelated sequence. The differences in the nature and intensity of the complex formed in the presence of specific and nonspecific competitor allows identification of specific interactions. Refer to Promega, Gel Shift Assay FAQ, available at <http://www.promega.com/faq/gelshfaq.html> (last visited Mar. 25, 2005), which is herein incorporated by reference in its entirety for teachings regarding gel shift methods.

Gel shift methods can include using, for example, colloidal forms of COOMASSIE (Imperial Chemicals Industries, Ltd) blue stain to detect proteins in gels such as polyacrylamide electrophoresis gels. Such methods are described, for example, in Neuhoff et al., Electrophoresis 6:427-448 (1985), and Neuhoff et al., Electrophoresis 9:255-262 (1988), each of which is herein incorporated by reference in its entirety for teachings regarding gel shift methods. In addition to the conventional protein assay methods referenced above, a combination cleaning and protein staining composition is described in U.S. Pat. No. 5,424,000, herein incorporated by reference in its entirety for its teaching regarding gel shift methods. The solutions can include phosphoric, sulfuric, and nitric acids, and Acid Violet dye.

Radioimmune Precipitation Assay (RIPA) is a sensitive assay using radiolabeled antigens to detect specific antibodies in serum. The antigens are allowed to react with the serum and then precipitated using a special reagent such as, for example, protein A sepharose beads. The bound radiolabeled immunoprecipitate is then commonly analyzed by gel electrophoresis. Radioimmunoprecipitation assay (RIPA) is often used as a confirmatory test for diagnosing the presence of HIV antibodies. RIPA is also referred to in the art as Farr Assay, Precipitin Assay, Radioimmune Precipitin Assay; Radioimmunoprecipitation Analysis; Radioimmunoprecipitation Analysis, and Radioimmunoprecipitation Analysis.

While the above immunoassays that utilize electrophoresis to separate and detect the specific proteins of interest allow for evaluation of protein size, they are not very sensitive for evaluating protein concentration. However, also contemplated are immunoassays wherein the protein or antibody specific for the protein is bound to a solid support (e.g., tube, well, bead, or cell) to capture the antibody or protein of interest, respectively, from a sample, combined with a method of detecting the protein or antibody specific for the protein on the support. Examples of such immunoassays include Radioimmunoassay (RIA), Enzyme-Linked Immunosorbent Assay (ELISA), Flow cytometry, protein array, multiplexed bead assay, and magnetic capture.

Radioimmunoassay (RIA) is a classic quantitative assay for detection of antigen-antibody reactions using a radioactively labeled substance (radioligand), either directly or indirectly, to measure the binding of the unlabeled substance to a specific antibody or other receptor system. Radioimmunoassay is used, for example, to test hormone levels in the blood without the need to use a bioassay. Non-immunogenic substances (e.g., haptens) can also be measured if coupled to larger carrier proteins (e.g., bovine gamma-globulin or human serum albumin) capable of inducing antibody formation. RIA involves mixing a radioactive antigen (because of the ease with which iodine atoms can be introduced into tyrosine residues in a protein, the radioactive isotopes ¹²⁵I or ¹³¹I are often used) with antibody to that antigen. The antibody is generally linked to a solid support, such as a tube or beads. Unlabeled or “cold” antigen is then adding in known quantities and measuring the amount of labeled antigen displaced. Initially, the radioactive antigen is bound to the antibodies. When cold antigen is added, the two compete for antibody binding sites—and at higher concentrations of cold antigen, more binds to the antibody, displacing the radioactive variant. The bound antigens are separated from the unbound ones in solution and the radioactivity of each used to plot a binding curve. The technique is both extremely sensitive, and specific.

Enzyme-Linked Immunosorbent Assay (ELISA), or more generically termed EIA (Enzyme ImmunoAssay), is an immunoassay that can detect an antibody specific for a protein. In such an assay, a detectable label bound to either an antibody-binding or antigen-binding reagent is an enzyme. When exposed to its substrate, this enzyme reacts in such a manner as to produce a chemical moiety which can be detected, for example, by spectrophotometric, fluorometric or visual means. Enzymes which can be used to detectably label reagents useful for detection include, but are not limited to, horseradish peroxidase, alkaline phosphatase, glucose oxidase, β-galactosidase, ribonuclease, urease, catalase, malate dehydrogenase, staphylococcal nuclease, asparaginase, yeast alcohol dehydrogenase, α-glycerophosphate dehydrogenase, triose phosphate isomerase, glucose-6-phosphate dehydrogenase, glucoamylase and acetylcholinesterase. For descriptions of ELISA procedures, see Voller, A. et al., J. Clin. Pathol. 31:507-520 (1978); Butler, J. E., Meth. Enzymol. 73:482-523 (1981); Maggio, E. (ed.), Enzyme Immunoassay, CRC Press, Boca Raton, 1980; Butler, J. E., In: Structure of Antigens, Vol. 1 (Van Regenmortel, M., CRC Press, Boca Raton, 1992, pp. 209-259; Butler, J. E., In: van Oss, C. J. et al., (eds), Immunochemistry, Marcel Dekker, Inc., New York, 1994, pp. 759-803; Butler, J. E. (ed.), Immunochemistry of Solid-Phase Immunoassay, CRC Press, Boca Raton, 1991); Crowther, “ELISA: Theory and Practice,” In: Methods in Molecule Biology, Vol. 42, Humana Press; New Jersey, 1995; U.S. Pat. No. 4,376,110, each of which is incorporated herein by reference in its entirety and specifically for teachings regarding ELISA methods.

Variations of ELISA techniques are know to those of skill in the art. In one variation, antibodies that can bind to proteins can be immobilized onto a selected surface exhibiting protein affinity, such as a well in a polystyrene microtiter plate. Then, a test composition suspected of containing a marker antigen can be added to the wells. After binding and washing to remove non-specifically bound immunocomplexes, the bound antigen can be detected. Detection can be achieved by the addition of a second antibody specific for the target protein, which is linked to a detectable label. This type of ELISA is a simple “sandwich ELISA.” Detection also can be achieved by the addition of a second antibody, followed by the addition of a third antibody that has binding affinity for the second antibody, with the third antibody being linked to a detectable label.

Another variation is a competition ELISA. In competition ELISAs, test samples compete for binding with known amounts of labeled antigens or antibodies. The amount of reactive species in the sample can be determined by mixing the sample with the known labeled species before or during incubation with coated wells. The presence of reactive species in the sample acts to reduce the amount of labeled species available for binding to the well and thus reduces the ultimate signal.

Regardless of the format employed, ELISAs have certain features in common, such as coating, incubating or binding, washing to remove non-specifically bound species, and detecting the bound immunecomplexes. Antigen or antibodies can be linked to a solid support, such as in the form of plate, beads, dipstick, membrane or column matrix, and the sample to be analyzed applied to the immobilized antigen or antibody. In coating a plate with either antigen or antibody, one will generally incubate the wells of the plate with a solution of the antigen or antibody, either overnight or for a specified period of hours. The wells of the plate can then be washed to remove incompletely adsorbed material. Any remaining available surfaces of the wells can then be “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, a secondary or tertiary detection means rather than a direct procedure can also be used. Thus, after binding of a protein or antibody to the well, coating with a non-reactive material to reduce background, and washing to remove unbound material, the immobilizing surface is contacted with the control clinical or biological sample to be tested under conditions effective to allow immunecomplex (antigen/antibody) formation. Detection of the immunecomplex then requires a labeled secondary binding agent or a secondary binding agent in conjunction with a labeled third binding agent.

“Under conditions effective to allow immunecomplex (antigen/antibody) formation” means that the conditions include diluting the antigens and antibodies with solutions such as BSA, bovine gamma globulin (BGG) and phosphate buffered saline (PBS)/Tween so as to reduce non-specific binding and to promote a reasonable signal to noise ratio.

The suitable conditions also mean that the incubation is at a temperature and for a period of time sufficient to allow effective binding. Incubation steps can typically be from about 1 minute to twelve hours, at temperatures of about 20° to 30° C., or can be incubated overnight at about 0° C. to about 10° C.

Following all incubation steps in an ELISA, the contacted surface can be washed so as to remove non-complexed material. A washing procedure can include washing with a solution such as PBS/Tween or borate buffer. Following the formation of specific immunecomplexes between the test sample and the originally bound material, and subsequent washing, the occurrence of even minute amounts of immunecomplexes can be determined

To provide a detecting means, the second or third antibody can have an associated label to allow detection, as described above. This can be an enzyme that can generate color development upon incubating with an appropriate chromogenic substrate. Thus, for example, one can contact and incubate the first or second immunecomplex with a labeled antibody for a period of time and under conditions that favor the development of further immunecomplex formation (e.g., incubation for 2 hours at room temperature in a PBS-containing solution such as PBS-Tween).

After incubation with the labeled antibody, and subsequent to washing to remove unbound material, the amount of label can be quantified, e.g., by incubation with a chromogenic substrate such as urea and bromocresol purple or 2,2′-azido-di-(3-ethyl-benzthiazoline-6-sulfonic acid [ABTS] and H₂O₂, in the case of peroxidase as the enzyme label. Quantitation can then be achieved by measuring the degree of color generation, e.g., using a visible spectra spectrophotometer.

Protein arrays are solid-phase ligand binding assay systems using immobilized proteins on surfaces which include glass, membranes, microtiter wells, mass spectrometer plates, and beads or other particles. The assays are highly parallel (multiplexed) and often miniaturized (microarrays, protein chips). Their advantages include being rapid and automatable, capable of high sensitivity, economical on reagents, and giving an abundance of data for a single experiment. Bioinformatics support is important; the data handling demands sophisticated software and data comparison analysis. However, the software can be adapted from that used for DNA arrays, as can much of the hardware and detection systems.

One of the chief formats is the capture array, in which ligand-binding reagents, which are usually antibodies but can also be alternative protein scaffolds, peptides or nucleic acid aptamers, are used to detect target molecules in mixtures such as plasma or tissue extracts. In diagnostics, capture arrays can be used to carry out multiple immunoassays in parallel, both testing for several analytes in individual sera for example and testing many serum samples simultaneously. In proteomics, capture arrays are used to quantitate and compare the levels of proteins in different samples in health and disease, i.e. protein expression profiling. Proteins other than specific ligand binders are used in the array format for in vitro functional interaction screens such as protein-protein, protein-DNA, protein-drug, receptor-ligand, enzyme-substrate, etc. The capture reagents themselves are selected and screened against many proteins, which can also be done in a multiplex array format against multiple protein targets.

For construction of arrays, sources of proteins include cell-based expression systems for recombinant proteins, purification from natural sources, production in vitro by cell-free translation systems, and synthetic methods for peptides. Many of these methods can be automated for high throughput production. For capture arrays and protein function analysis, it is important that proteins should be correctly folded and functional; this is not always the case, e.g. where recombinant proteins are extracted from bacteria under denaturing conditions. Nevertheless, arrays of denatured proteins are useful in screening antibodies for cross-reactivity, identifying autoantibodies and selecting ligand binding proteins.

Protein arrays have been designed as a miniaturization of familiar immunoassay methods such as ELISA and dot blotting, often utilizing fluorescent readout, and facilitated by robotics and high throughput detection systems to enable multiple assays to be carried out in parallel. Commonly used physical supports include glass slides, silicon, microwells, nitrocellulose or PVDF membranes, and magnetic and other microbeads. While microdrops of protein delivered onto planar surfaces are the most familiar format, alternative architectures include CD centrifugation devices based on developments in microfluidics (Gyros, Monmouth Junction, N.J.) and specialised chip designs, such as engineered microchannels in a plate (e.g., The Living Chip™, Biotrove, Woburn, Mass.) and tiny 3D posts on a silicon surface (Zyomyx, Hayward Calif.). Particles in suspension can also be used as the basis of arrays, providing they are coded for identification; systems include colour coding for microbeads (Luminex, Austin, Tex.; Bio-Rad Laboratories) and semiconductor nanocrystals (e.g., QDots™, Quantum Dot, Hayward, Calif.), and barcoding for beads (UltraPlex™, SmartBead Technologies Ltd, Babraham, Cambridge, UK) and multimetal microrods (e.g., Nanobarcodes™ particles, Nanoplex Technologies, Mountain View, Calif.). Beads can also be assembled into planar arrays on semiconductor chips (LEAPS technology, BioArray Solutions, Warren, N.J.).

Immobilization of proteins involves both the coupling reagent and the nature of the surface being coupled to. A good protein array support surface is chemically stable before and after the coupling procedures, allows good spot morphology, displays minimal nonspecific binding, does not contribute a background in detection systems, and is compatible with different detection systems. The immobilization method used are reproducible, applicable to proteins of different properties (size, hydrophilic, hydrophobic), amenable to high throughput and automation, and compatible with retention of fully functional protein activity. Orientation of the surface-bound protein is recognized as an important factor in presenting it to ligand or substrate in an active state; for capture arrays the most efficient binding results are obtained with orientated capture reagents, which generally require site-specific labeling of the protein.

Both covalent and noncovalent methods of protein immobilization are used and have various pros and cons. Passive adsorption to surfaces is methodologically simple, but allows little quantitative or orientational control; it may or may not alter the functional properties of the protein, and reproducibility and efficiency are variable. Covalent coupling methods provide a stable linkage, can be applied to a range of proteins and have good reproducibility; however, orientation may be variable, chemical derivatization may alter the function of the protein and requires a stable interactive surface. Biological capture methods utilizing a tag on the protein provide a stable linkage and bind the protein specifically and in reproducible orientation, but the biological reagent must first be immobilized adequately and the array may require special handling and have variable stability.

Several immobilization chemistries and tags have been described for fabrication of protein arrays. Substrates for covalent attachment include glass slides coated with amino- or aldehyde-containing silane reagents. In the Versalinx™ system (Prolinx, Bothell, Wash.) reversible covalent coupling is achieved by interaction between the protein derivatised with phenyldiboronic acid, and salicylhydroxamic acid immobilized on the support surface. This also has low background binding and low intrinsic fluorescence and allows the immobilized proteins to retain function. Noncovalent binding of unmodified protein occurs within porous structures such as HydroGel™ (PerkinElmer, Wellesley, Mass.), based on a 3-dimensional polyacrylamide gel; this substrate is reported to give a particularly low background on glass microarrays, with a high capacity and retention of protein function. Widely used biological coupling methods are through biotin/streptavidin or hexahistidine/Ni interactions, having modified the protein appropriately. Biotin may be conjugated to a poly-lysine backbone immobilised on a surface such as titanium dioxide (Zyomyx) or tantalum pentoxide (Zeptosens, Witterswil, Switzerland).

Array fabrication methods include robotic contact printing, ink-jetting, piezoelectric spotting and photolithography. A number of commercial arrayers are available [e.g. Packard Biosciences] as well as manual equipment [V & P Scientific]. Bacterial colonies can be robotically gridded onto PVDF membranes for induction of protein expression in situ.

At the limit of spot size and density are nanoarrays, with spots on the nanometer spatial scale, enabling thousands of reactions to be performed on a single chip less than 1 mm square. BioForce Laboratories have developed nanoarrays with 1521 protein spots in 85 sq microns, equivalent to 25 million spots per sq cm, at the limit for optical detection; their readout methods are fluorescence and atomic force microscopy (AFM).

Fluorescence labeling and detection methods are widely used. The same instrumentation as used for reading DNA microarrays is applicable to protein arrays. For differential display, capture (e.g., antibody) arrays can be probed with fluorescently labeled proteins from two different cell states, in which cell lysates are directly conjugated with different fluorophores (e.g. Cy-3, Cy-5) and mixed, such that the color acts as a readout for changes in target abundance. Fluorescent readout sensitivity can be amplified 10-100 fold by tyramide signal amplification (TSA) (PerkinElmer Lifesciences). Planar waveguide technology (Zeptosens) enables ultrasensitive fluorescence detection, with the additional advantage of no intervening washing procedures. High sensitivity can also be achieved with suspension beads and particles, using phycoerythrin as label (Luminex) or the properties of semiconductor nanocrystals (Quantum Dot). A number of novel alternative readouts have been developed, especially in the commercial biotech arena. These include adaptations of surface plasmon resonance (HTS Biosystems, Intrinsic Bioprobes, Tempe, Ariz.), rolling circle DNA amplification (Molecular Staging, New Haven Conn.), mass spectrometry (Intrinsic Bioprobes; Ciphergen, Fremont, Calif.), resonance light scattering (Genicon Sciences, San Diego, Calif.) and atomic force microscopy [BioForce Laboratories].

Capture arrays form the basis of diagnostic chips and arrays for expression profiling. They employ high affinity capture reagents, such as conventional antibodies, single domains, engineered scaffolds, peptides or nucleic acid aptamers, to bind and detect specific target ligands in high throughput manner.

Antibody arrays have the required properties of specificity and acceptable background, and some are available commercially (BD Biosciences, San Jose, Calif.; Clontech, Mountain View, Calif.; BioRad; Sigma, St. Louis, Mo.). Antibodies for capture arrays are made either by conventional immunization (polyclonal sera and hybridomas), or as recombinant fragments, usually expressed in E. coli, after selection from phage or ribosome display libraries (Cambridge Antibody Technology, Cambridge, UK; BioInvent, Lund, Sweden; Affitech, Walnut Creek, Calif.; Biosite, San Diego, Calif.). In addition to the conventional antibodies, Fab and scFv fragments, single V-domains from camelids or engineered human equivalents (Domantis, Waltham, Mass.) may also be useful in arrays.

The term “scaffold” refers to ligand-binding domains of proteins, which are engineered into multiple variants capable of binding diverse target molecules with antibody-like properties of specificity and affinity. The variants can be produced in a genetic library format and selected against individual targets by phage, bacterial or ribosome display. Such ligand-binding scaffolds or frameworks include ‘Affibodies’ based on Staph. aureus protein A (Affibody, Bromma, Sweden), ‘Trinectins’ based on fibronectins (Phylos, Lexington, Mass.) and ‘Anticalins’ based on the lipocalin structure (Pieris Proteolab, Freising-Weihenstephan, Germany). These can be used on capture arrays in a similar fashion to antibodies and may have advantages of robustness and ease of production.

Nonprotein capture molecules, notably the single-stranded nucleic acid aptamers which bind protein ligands with high specificity and affinity, are also used in arrays (SomaLogic, Boulder, Colo.). Aptamers are selected from libraries of oligonucleotides by the Selex™ procedure and their interaction with protein can be enhanced by covalent attachment, through incorporation of brominated deoxyuridine and UV-activated crosslinking (photoaptamers). Photocrosslinking to ligand reduces the crossreactivity of aptamers due to the specific steric requirements. Aptamers have the advantages of ease of production by automated oligonucleotide synthesis and the stability and robustness of DNA; on photoaptamer arrays, universal fluorescent protein stains can be used to detect binding.

Protein analytes binding to antibody arrays may be detected directly or via a secondary antibody in a sandwich assay. Direct labelling is used for comparison of different samples with different colours. Where pairs of antibodies directed at the same protein ligand are available, sandwich immunoassays provide high specificity and sensitivity and are therefore the method of choice for low abundance proteins such as cytokines; they also give the possibility of detection of protein modifications. Label-free detection methods, including mass spectrometry, surface plasmon resonance and atomic force microscopy, avoid alteration of ligand. What is required from any method is optimal sensitivity and specificity, with low background to give high signal to noise. Since analyte concentrations cover a wide range, sensitivity has to be tailored appropriately; serial dilution of the sample or use of antibodies of different affinities are solutions to this problem. Proteins of interest are frequently those in low concentration in body fluids and extracts, requiring detection in the pg range or lower, such as cytokines or the low expression products in cells.

An alternative to an array of capture molecules is one made through ‘molecular imprinting’ technology, in which peptides (e.g., from the C-terminal regions of proteins) are used as templates to generate structurally complementary, sequence-specific cavities in a polymerizable matrix; the cavities can then specifically capture (denatured) proteins that have the appropriate primary amino acid sequence (ProteinPrint™, Aspira Biosystems, Burlingame, Calif.).

Another methodology which can be used diagnostically and in expression profiling is the ProteinChip® array (Ciphergen, Fremont, Calif.), in which solid phase chromatographic surfaces bind proteins with similar characteristics of charge or hydrophobicity from mixtures such as plasma or tumour extracts, and SELDI-TOF mass spectrometry is used to detection the retained proteins.

Large-scale functional chips have been constructed by immobilizing large numbers of purified proteins and used to assay a wide range of biochemical functions, such as protein interactions with other proteins, drug-target interactions, enzyme-substrates, etc. Generally they require an expression library, cloned into E. coli, yeast or similar from which the expressed proteins are then purified, e.g. via a His tag, and immobilized. Cell free protein transcription/translation is a viable alternative for synthesis of proteins which do not express well in bacterial or other in vivo systems.

For detecting protein-protein interactions, protein arrays can be in vitro alternatives to the cell-based yeast two-hybrid system and may be useful where the latter is deficient, such as interactions involving secreted proteins or proteins with disulphide bridges. High-throughput analysis of biochemical activities on arrays has been described for yeast protein kinases and for various functions (protein-protein and protein-lipid interactions) of the yeast proteome, where a large proportion of all yeast open-reading frames was expressed and immobilised on a microarray. Large-scale ‘proteome chips’ promise to be very useful in identification of functional interactions, drug screening, etc. (Proteometrix, Branford, Conn.).

As a two-dimensional display of individual elements, a protein array can be used to screen phage or ribosome display libraries, in order to select specific binding partners, including antibodies, synthetic scaffolds, peptides and aptamers. In this way, ‘library against library’ screening can be carried out. Screening of drug candidates in combinatorial chemical libraries against an array of protein targets identified from genome projects is another application of the approach.

A multiplexed bead assay, such as, for example, the BD™ Cytometric Bead Array, is a series of spectrally discrete particles that can be used to capture and quantitate soluble analytes. The analyte is then measured by detection of a fluorescence-based emission and flow cytometric analysis. Multiplexed bead assay generates data that is comparable to ELISA based assays, but in a “multiplexed” or simultaneous fashion. Concentration of unknowns is calculated for the cytometric bead array as with any sandwich format assay, i.e. through the use of known standards and plotting unknowns against a standard curve. Further, multiplexed bead assay allows quantification of soluble analytes in samples never previously considered due to sample volume limitations. In addition to the quantitative data, powerful visual images can be generated revealing unique profiles or signatures that provide the user with additional information at a glance.

The biomarker profiles disclosed herein are based on measurements of individual biomarkers. The amounts of these can be measured by any means known to provide an acceptable indication of how much of any of these is present in the sample being analyzed. An example of a means of measuring is provided in the Examples. The process of measuring an amount of an analyte (e.g., MPP, TIMP, PINP, PIIINP, CITP, cardiotrophin, sRAGE, osteopontin) includes measurement of no amount or an undetectable amount of the analyte.

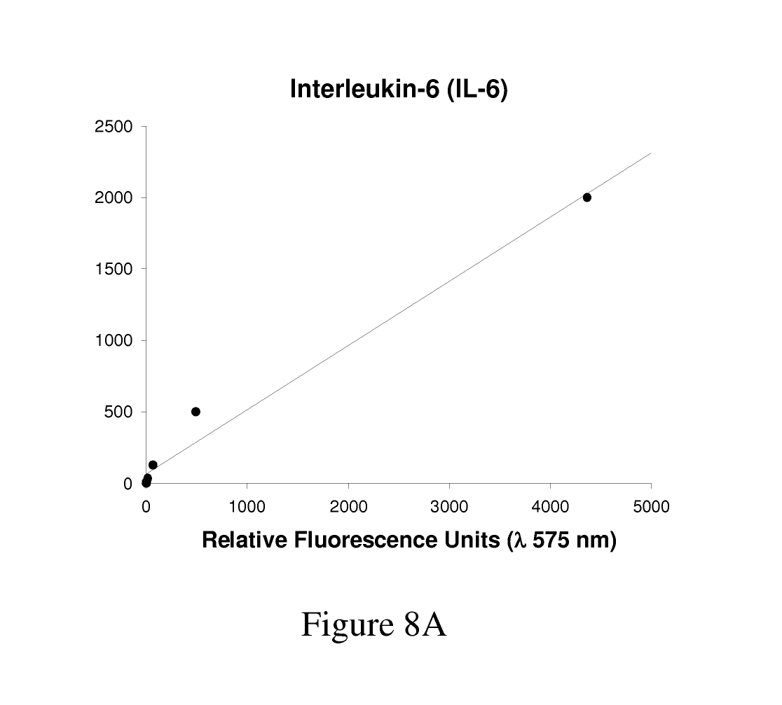

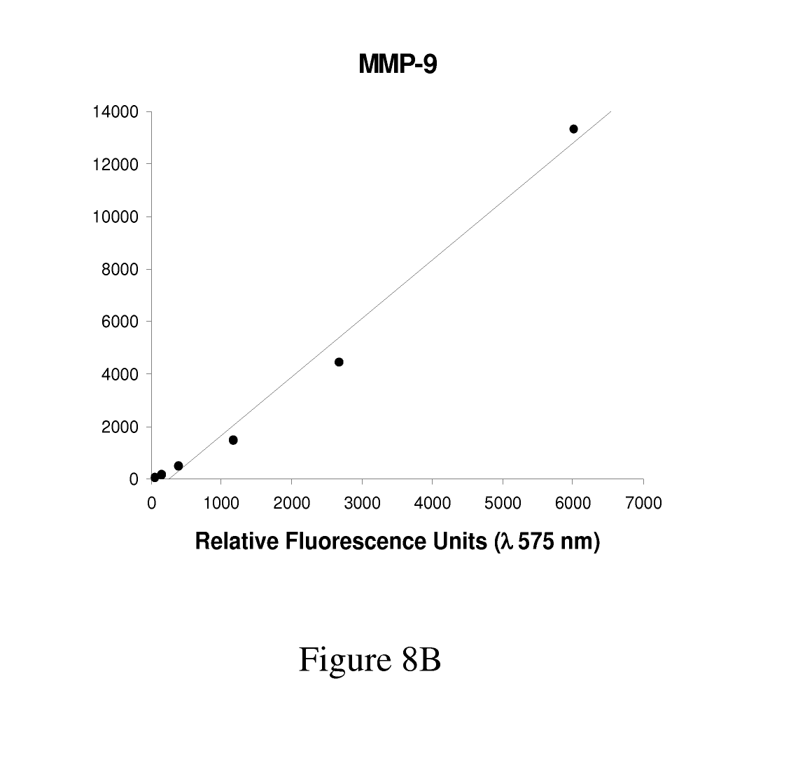

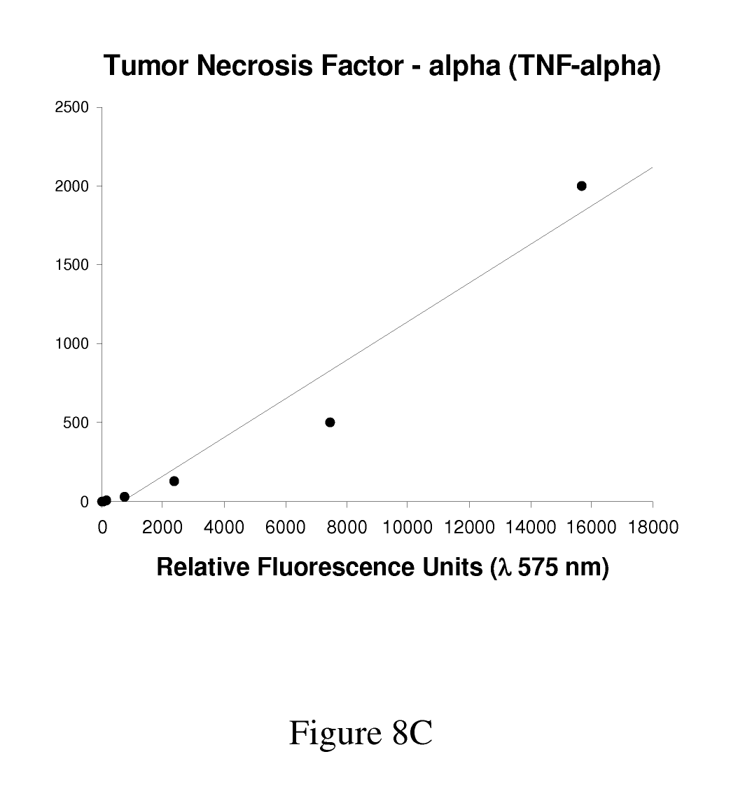

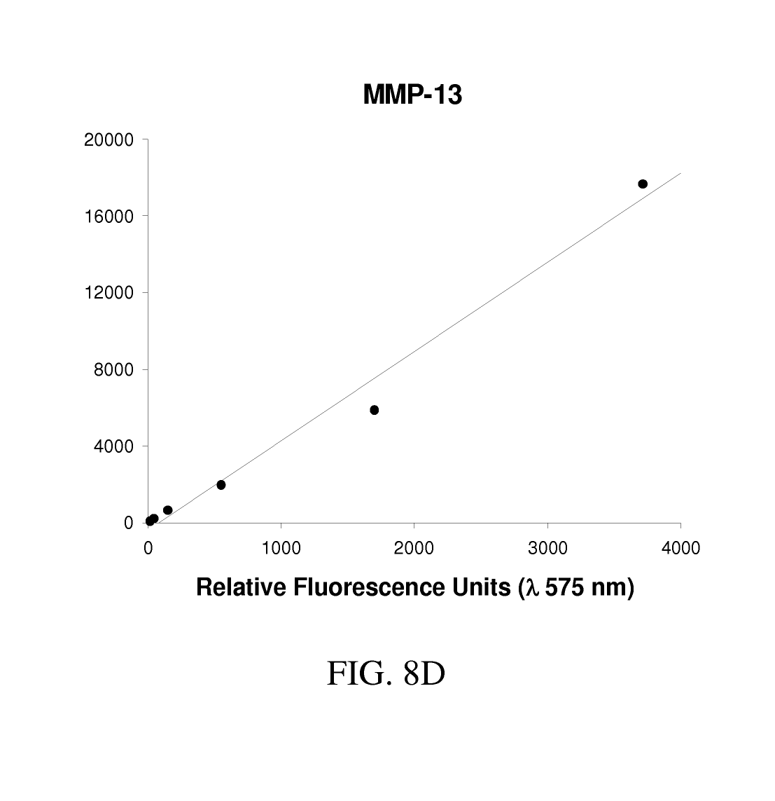

The techniques and approaches for measuring biomarkers which can be used with the disclosed methods can be based upon high sensitivity immunoassays. The immunoassay approach which was standardized for providing the measurements shown in Table 1 was performed by an enzyme linked immuno-assay (ELISA). However, other more sensitive and rapid methods for measuring blood levels of MMPs, TIMPs, and other biomarkers can also be used and these include the use of a multiplex assay system. In this example, multiple analytes in volume-limited samples, such as plasma or other biological samples, can be measured using a bead-based multiplex sandwich immunoassay. This emergent technique for multiplex analysis is built on technology that combines the sensitivity of ELISA with flow cytometric detection, allowing for the specific measurement of up to 100 different analytes within a single sample of less than 50 μl. This approach allows for the measurement of multiple biomarkers in a small blood sample. This type of approach is well-suited for the diagnostic, prognostic, predictive and therapeutic monitoring applications that are described herein. Specifically, to measure analyte concentrations simultaneously, the microbeads are incubated with sample (e.g., blood sample) and allowed to form complexes with the specific analytes of interest (e.g., MMPs). Detection antibodies (biotinylated), specific for a second epitope on each analyte, are then added to the mixture and allowed to bind to the microbeads complexed with analyte. The mixture is then incubated with a fluorescent reporter molecule (streptavidin-phycoerythrin) and the entire sample is passed through a two-laser flow cytometric detector. One laser detects the precise fluorescence of the microbead which defines the specific analyte being examined, and the other laser detects the amount of reporter fluorescence which is directly proportional to the amount of analyte bound. This process has been applied to a number of MMPs and other analytes that are related to the CHF process and these are shown in FIG. 8 and Table 1. This is but one example of how single or multiple analytes can be measured with a very small blood sample. Other examples of measurements that have been performed with respect to MMPs, TIMPs, and other biomarkers include radioimmunoassay and immunoblotting assays. These approaches are also antibody based.

TABLE 1
Concentration range of analytes used for calibration and
linear regression statistics for calculated standard curves.

| Analyte | Range (pg/ml) | R² | P-value |
|---|---|---|---|
| MMP-1 | 14.1-3433.33 | 0.96 | 0.0004 |
| MMP-2 | 75.5-18333.33 | 0.99 | 0.0001 |
| MMP-3 | 13.0-3166.67 | 0.97 | 0.0002 |
| MMP-7 | 96.0-23333.33 | 0.98 | 0.0001 |
| MMP-8 | 83.7-20333.33 | 0.96 | 0.0004 |
| MMP-9 | 54.9-13333.33 | 0.98 | 0.0001 |
| MMP-12 | 12.8-31000.00 | 0.97 | 0.0003 |
| MMP-13 | 72.7-17666.70 | 0.98 | 0.0001 |
| TNF-alpha | 1.95-2000.0 | 0.95 | 0.0002 |
| IL-1 beta | 1.95-2000.0 | 0.94 | 0.0002 |
| IL-2 | 1.95-2000.0 | 0.98 | 0.0001 |
| IL-6 | 1.95-2000.0 | 0.98 | 0.0001 |
| IL-8 | 1.95-2000.0 | 0.91 | 0.0007 |
| IL-10 | 1.95-2000.0 | 0.97 | 0.0001 |
| G-CSF | 1.95-2000.0 | 0.99 | 0.0001 |
| INF-gamma | 1.95-2000.0 | 0.99 | 0.0001 |
| MCP-1 | 1.95-2000.0 | 0.96 | 0.0001 |
| MIP-beta | 1.95-2000.0 | 0.91 | 0.0008 |

### D. Reference Values

Provided are profiles of biomarkers that are indicative of the existence of LVH, CHF, and/or DHF or are predictive of the development of LVH, CHF, and/or DHF in a subject. The profiles that are indicative of the existence of LVH, CHF, and/or DHF or are predictive of the development of LVH, CHF, and/or DHF in a subject can be relative to a reference value. The reference value can be an amount or concentration of a biomarker. In such cases the reference value can be referred to as a reference amount. For example, the reference amount can be a normal value or amount, the amount in a normal subject, the amount in a subject with hypertension, or the amount in a subject with left ventricular hypertrophy. A normal value or amount for a given analyte can be, for example, a reference value or amount for an age matched subject that is confirmed to have no evidence of significant cardiovascular disease. Thus, the normal value or amount can be a population-based value derived from a significant number of healthy individuals. These reference normal values can be obtained from population based studies. There are large population based studies for example that have identified relative levels of TIMP-1 (Framingham Heart Study, Circulation 2004; 109:2850-2856) in a reference group to approximately 800 ng/mL which is consistent with the reference control values disclosed herein.

Reference values can differ based on the assay system and reagents used. For example, RIA, EISHA, MIA, etc. systems may provide different amounts for a given biomarker from the same sample or population of samples. Thus, it is understood that reference values generally should be determined using the same or similar assays and reagents as will be used for measuring amounts in samples when performing the disclosed methods. For this reason, it is contemplated and should be understood that the direction of the change, the magnitude of the change, or both, of a biomarker or ratio of biomarkers should be used to compare reference amounts and the assay amounts measured in samples (see, for example, Tables 24 and 25). However, also disclosed herein are reference amounts of biomarkers in actual terms (such as, for example, ng/ml) determined using certain assay systems. Such reference amounts can be used when performing the disclosed methods, preferably when the same or similar assay system is being used.

Alternatively, the normal value or amount can be a value that is considered normal for a given subject. For example, baseline measurements of the relevant analytes can be made for a healthy individual, and used for comparison against later-acquired measurements from that individual to identify current disease or progression toward hypertensive heart disease.

A discrete observation, e.g., for MMP-13, is where a continuous variable such as a plasma concentration of a given analyte is converted to a dichotomous variable. In this particular instance a +/− value would be assigned to MMP-13 where a value of greater than 10 ng/mL would be considered a detectable, or positive value and a value less than 10 ng/mL to be a negative value. Other discrete biomarkers are gender and ethnicity.

For example, provided is a method of diagnosing the absence of LVH associated with hypertensive heart disease in a subject comprising measuring biomarker levels in a tissue or bodily fluid of the subject and comparing the levels to reference values. Thus, normal values for MMP-2, MMP-3, MMP-9, MMP-7, MMP-13, MMP-8, TIMP-1, TIMP-2, TIMP-4, NTBNP, PIIINP, CITP, osteopontin, cardiotrophin, and/or sRAGE is an indication of the absence of left ventricular hypertrophy associated with hypertensive heart disease.

In some aspects, MMP-2 plasma levels within normal range is an indication of the absence of LVH associated with hypertensive heart disease. In some aspects, MMP-3 plasma levels within normal range is an indication of the absence of LVH associated with hypertensive heart disease. In some aspects, MMP-9 plasma levels within normal range is an indication of the absence of LVH associated with hypertensive heart disease. In some aspects, MMP-13 plasma levels within normal range is an indication of the absence of LVH associated with hypertensive heart disease. In some aspects, TIMP-1 plasma levels within normal range is an indication of the absence of LVH associated with hypertensive heart disease. In some aspects, TIMP-2 plasma levels within normal range is an indication of the absence of LVH associated with hypertensive heart disease. In some aspects, TIMP-4 plasma levels within normal range is an indication of the absence of LVH associated with hypertensive heart disease. In some aspects, NTBNP plasma levels within normal range is an indication of the absence of LVH associated with hypertensive heart disease. In some aspects, PIIINP plasma levels within normal range is an indication of the absence of LVH associated with hypertensive heart disease. In some aspects, CITP plasma levels within normal range is an indication of the absence of LVH associated with hypertensive heart disease. In some aspects, osteopontin plasma levels within normal range is an indication of the absence of LVH associated with hypertensive heart disease. In some aspects, cardiotrophin plasma levels within normal range is an indication of the absence of LVH associated with hypertensive heart disease. In some aspects, sRAGE plasma levels within normal range is an indication of the absence of LVH associated with hypertensive heart disease.

In some aspects, MMP-2 plasma levels greater than about 1000 ng/ml, including greater than about 1000, 1100, 1200, 1300, 1400, and 1500 ng/ml, is an indication of the absence of LVH associated with hypertensive heart disease.

In some aspects, MMP-3 plasma levels greater than about 10 ng/ml, including greater than about 10, 11, 12, 13, 14, and 15 ng/ml, is an indication of the absence of LVH associated with hypertensive heart disease.

In some aspects, MMP-9 plasma levels less than about 20 ng/ml, including less than about 20, 19, 18, 17, 16, 15, 14, 13, 12, 11, 10, 9, 8, 7, 6, 5, 4, 3, 2, or 1 ng/ml, is an indication of the absence of LVH associated with hypertensive heart disease.

In some aspects, detectable MMP-13 plasma levels greater than about 5 ng/ml, including less than about 5, 6, 7, 8, 9, 10, 11, 12, 13, 14, 15, 20 ng/ml, is an indication of the absence of LVH associated with hypertensive heart disease.

In some aspects, TIMP-1 plasma levels less than about 1000 ng/ml, including greater than about 1000, 900, 800, 700, 600, 500, 400, 300, 200, 100, 50, 40, 30, 20, or 10 ng/ml, is an indication of the absence of LVH associated with hypertensive heart disease.

In some aspects, TIMP-2 plasma levels less than about 50 ng/ml, including greater than about 50, 49, 48, 47, 46, 45, 44, 43, 42, 41, 40, 35, 30, 25, 20, 15, or 10 ng/ml, is an indication of the absence of LVH associated with hypertensive heart disease.

In some aspects, TIMP-4 plasma levels less than about 2 ng/ml, including greater than about 2.0, 1.9, 1.8, 1.7, 1.6, 1.5, 1.4, 1.3, 1.2, 1.1, 1.0, 0.5, or 0.1 ng/ml, is an indication of the absence of LVH associated with hypertensive heart disease.

In some aspects, PIIINP plasma levels less than about 8 ng/ml, including greater than about 8, 7.5, 7, 6.5, 6, and 5.5 ng/ml, is an indication of the absence of LVH associated with hypertensive heart disease.

In some aspects, CITP plasma levels less than about 2 ng/ml, including greater than about 2, 1.8, 1.6, 1.5, 1.4, and 1.3 ng/ml, is an indication of the absence of LVH associated with hypertensive heart disease.

In some aspects, NTBNP plasma levels less than about 150 ng/ml, including greater than about 150, 140, 130, 120, 110, 100, 95, and 90 ng/ml, is an indication of the absence of LVH associated with hypertensive heart disease.

In some aspects, osteopontin plasma levels less than about 80 ng/ml, including greater than about 80, 75, 70, 65, and 60 ng/ml, is an indication of the absence of LVH associated with hypertensive heart disease.

In some aspects, cardiotrophin plasma levels greater than about 20 ng/ml, including greater than about 20, 21, 22, 23, 24, 25, 26, 27, 28, and 29 ng/ml, is an indication of the absence of LVH associated with hypertensive heart disease.

In some aspects, sRAGE plasma levels greater than about 2.6 ng/ml, including greater than about 2.6, 2.7, 2.8, 2.9, and 3.0 ng/ml, is an indication of the absence of LVH associated with hypertensive heart disease.

The method can further comprise calculating the ratio of one or more of the biomarkers to other biomarkers. For example, the method can comprise calculating the ratio of MMP-9 to TIMP-1, TIMP-2 or TIMP-4 or the ratio of PIIINP to PINP or CITP.

For example, in some aspects, a ratio of MMP-9/TIMP-1 plasma levels greater than about 7×10³, including greater than about 7×10³, 8×10³, 9×10³, 10×10³, 11×10³, 12×10³, 13×10³, or 14×10³, is an indication of the absence of LVH associated with hypertensive heart disease.

In some aspects, a ratio of MMP-9/TIMP-2 plasma levels greater than about 10×10⁴, including greater than about 10×10⁴, 20×10⁴, 30×10⁴, or 40×10⁴, is an indication of the absence of LVH associated with hypertensive heart disease.

In some aspects, a ratio of MMP-9/TIMP-4 plasma levels greater than about 1, including greater than about 1, 2, 3, 4, 5, 6, 7, 8, or 9, is an indication of the absence of LVH associated with hypertensive heart disease.

In some aspects, a ratio of PIIINP/PINP plasma levels of greater than the normal value is an indication of LVH. For example, a ratio of PIIINP/PINP greater than at least about 0.28 is an indication of LVH. In some aspects, a ratio of PIIINP/PINP greater than at least about 0.28, 0.29, 0.30, 0.31, 0.32, 0.34, 0.36, 0.38, 0.40, 0.5, 0.6, 0.8, and 1.0 is an indication of LVH.

In some aspects, a ratio of PIIINP/CITP plasma levels of greater than the normal value is an indication of LVH. For example, a ratio of PIIINP/CITP greater than at least about 5.5 is an indication of LVH. In some aspects, a ratio of PIIINP/PINP greater than at least about 5.5, 5.6, 5.8, 6.0, 6.2, 6.5, 7.0, 7.5, 8.0, and 9.0 is an indication of LVH.

Some reference normal values and those measured at screening in hypertensive patients is shown in Table 3. In this instance, MMP-2 values may be reduced in hypertensive patients with LVH with no change in MMP-7 values. However, a discrete observation for MMP-13 will occur in that this will not be detected in hypertensive patients with LVH. Therefore a cutpoint of below 10 ng/mL would be considered a diagnostic criteria for hypertension and heart failure. TIMP-1 and TIMP-4 levels will be 50% higher in hypertensive patients with LVH compared to reference control values. The MMP-9/TIMP-4 ratio will be reduced by over 50% in hypertensive patients with LVH when compared to reference normal values.

TABLE 3
MMP and TIMP Data; Reference Normal Values and
Hypertensive Heart Disease; Diagnostic Percent Cutpoints

| | | Hypertension and | |
|---|---|---|---|
| | Normal | Failure | % change |
| MMP-2 (ng/mL) | 1387 ± 39 | 1205 ± 44* | ▾ 20% or |
| | | | greater |
| MMP-7 (ng/mL) | 2.5 ± 0.2 | similar to normal | NC |
| MMP-13 (ng/mL) | Detectable | Non-Detectable (<10) | Discrete value |
| MMP-9 (ng/mL) | 13 ± 3 | 26 ± 3* | ▴ 50% or |
| | | | greater |
| TIMP-1 (ng/mL) | 997 ± 36 | 1291 ± 70* | ▴ 50% or |
| | | | greater |
| TIMP-2 (ng/mL) | 44 ± 4 | 58 ± 7 | NC |
| TIMP-4 (ng/mL) | 1.9 ± 0.1 | 3.8 ± 0.1 | ▴ 50% or |
| | | | greater |
| MMP-9/TIMP-1 | 14 ± 3 | 15 ± 5 | NC |
| (×10⁻³) | | | |
| MMP-9/TIMP-2 | 388 ± 88 | 350 ± 250 | NC |
| (×10⁻³) | | | |
| MMP-9/TIMP-4 | 7.8 ± 1.6 | 2.52 ± 0.4* | ▾ 50% or |
| | | | greater |

NC = no change from Normal
*p < 0.05 vs. Normal

### E. Biomarker Profiles and Matrices

The disclosed biomarkers can be used in the disclosed methods in various combinations. Such combinations can be referred to as biomarker profiles and biomarker matrices. The disclosed methods benefit from using biomarker profiles and matrices because the concurrence of multiple factors indicating LVH, CHF and/or DHF or a risk of LVH, CHF and/or DHF can increase the reliability and accuracy of the indication and conclusion. Any combination of the disclosed biomarkers can be used. Further, any additional biomarkers or factors can also be used in combination with the disclosed biomarkers, combinations of biomarkers, and biomarker profiles and matrices. Any combination of two or more of the following biomarkers (and the following indications) can be used.

1. Examples of Biomarkers and Indications for Use in Biomarker Profiles and Matrices

The amount of PIIINP in a body fluid from the subject can be measured and an amount of PIIINP that is greater than the reference amount indicates the presence or risk of congestive heart failure in the subject. The amount of PIIINP can be at least about 10% greater than the reference amount. The amount of osteopontin in a body fluid from the subject can be measured and an amount of osteopontin that is greater than the reference amount indicates the presence or risk of congestive heart failure in the subject. The amount of osteopontin can be at least about 20% greater than the reference amount. The amount of cardiotrophin in a body fluid from the subject can be measured and an amount of cardiotrophin that is less than the reference amount indicates the presence or risk of congestive heart failure in the subject. The amount of cardiotrophin can be at least about 30% less than the reference amount. The amount of sRAGE in a body fluid from the subject can be measured and an amount of sRAGE that is less than the reference amount indicates the presence or risk of congestive heart failure in the subject. The amount of sRAGE can be at least about 10% less than the reference amount. The amount of MMP-2 in a body fluid from the subject can be measured and an amount of MMP-2 that is greater than the reference amount indicates the presence or risk of congestive heart failure in the subject. The amount of MMP-2 can be at least about 10% greater than the reference amount. The amount of MMP-2 in a body fluid from the subject can be measured and an amount of MMP-2 that is greater than the reference amount indicates the presence or risk of congestive heart failure in the subject. The amount of MMP-2 can be at least about 10% greater than the reference amount. The amount of MMP-7 in a body fluid from the subject can be measured and an amount of MMP-7 that is greater than the reference amount indicates the presence or risk of congestive heart failure in the subject. The amount of MMP-7 can be at least about 30% greater than the reference amount. The amount of TIMP-2 in a body fluid from the subject can be measured and an amount of TIMP-2 that is less than the reference amount indicates the presence or risk of congestive heart failure in the subject. The amount of TIMP-2 can be at least about 10% less than the reference amount. The amount of TIMP-2 in a body fluid from the subject can be measured and an amount of TIMP-2 that is less than the reference amount indicates the presence or risk of congestive heart failure in the subject. The amount of TIMP-2 can be at least about 10% less than the reference amount. The amount of CITP in a body fluid from the subject can be measured and an amount of CITP that is greater than the reference amount indicates the presence or risk of congestive heart failure in the subject. The amount of CITP can be at least about 20% greater than the reference amount. The amount of PIIINP and CITP in a body fluid from the subject can be measured and an increase in the ratio of PIIINP to CITP compared to a reference ratio indicates the presence or risk of congestive heart failure in the subject, wherein the reference ratio is the ratio in a normal subject. The reduction in the ratio of PIIINP to CITP can be at least about 10% compared to the reference ratio. The amount of PIIINP and PINP in a body fluid from the subject can be measured and an increase in the ratio of PIIINP to PINP compared to a reference ratio indicates the presence or risk of congestive heart failure in the subject, wherein the reference ratio is the ratio in a normal subject. The reduction in the ratio of PIIINP to PINP can be at least about 20% compared to the reference ratio.

The amount of MMP-13 in a body fluid from the subject can be measured and an amount of less than 10 ng/mL of MMP-13 indicates the presence or risk of congestive heart failure in the subject. The amount of TIMP-1 in a body fluid from the subject can be measured and an amount of TIMP-1 that is greater than the reference amount indicates the presence or risk of congestive heart failure in the subject. The amount of TIMP-1 can be at least about 20% greater than the reference amount. The amount of TIMP-4 in a body fluid from the subject can be measured and an amount of TIMP-4 that is greater than the reference amount indicates the presence or risk of congestive heart failure in the subject. The amount of TIMP-4 can be at least about 50% greater than the reference amount. The amount of MMP-8 in a body fluid from the subject can be measured and an amount of MMP-8 that is greater than the reference amount indicates the presence or risk of congestive heart failure in the subject. The amount of MMP-8 can be at least about 50% greater than the reference amount. The amount of MMP-9 in a body fluid from the subject can be measured and an amount of MMP-9 that is greater than the reference amount indicates the presence or risk of congestive heart failure in the subject. The amount of MMP-9 can be at least about 50% greater than the reference amount. The amount of MMP-9 and TIMP-1 in a body fluid from the subject can be measured and a reduction in the ratio of MMP-9 to TIMP-1 compared to a reference ratio indicates the presence or risk of congestive heart failure in the subject, wherein the reference ratio is the ratio in a normal subject. The reduction in the ratio of MMP-9 to TIMP-1 can be at least about 50% compared to the reference ratio. The amount of MMP-9 and TIMP-2 in a body fluid from the subject can be measured and a reduction in the ratio of MMP-9 to TIMP-2 compared to a reference ratio indicates the presence or risk of congestive heart failure in the subject, wherein the reference ratio is the ratio in a normal subject. The reduction in the ratio of MMP-9 to TIMP-2 can be at least about 50% compared to the reference ratio. The amount of MMP-9 and TIMP-4 in a body fluid from the subject can be measured and a reduction in the ratio of MMP-9 to TIMP-4 compared to a reference ratio indicates the presence or risk of congestive heart failure in the subject, wherein the reference ratio is the ratio in a normal subject. The reduction in the ratio of MMP-9 to TIMP-4 can be at least about 50% compared to the reference ratio. The amount of MMP-3 in a body fluid from the subject can be measured and an amount of MMP-3 that is greater than the reference amount indicates the presence or risk of congestive heart failure in the subject. The amount of MMP-3 can be at least about 50% greater than the reference amount. The amount of NTBNP in a body fluid from the subject can be measured and an amount of NTBNP that is greater than the reference amount indicates the presence or risk of congestive heart failure in the subject. The amount of NTBNP can be at least about 50% greater than the reference amount.

2. Examples of Biomarkers Combinations, Profiles, and Matrices

The method can further comprise measuring plasma levels of two or more biomarkers. For example, the method can comprise measuring two, three, four, five, six, seven, eight, nine, ten, eleven, twelve, thirteen, fourteen, fifteen, or sixteen of MMP-2, MMP-3, MMP-9, MMP-7, MMP-13, MMP-8, TIMP-1, TIMP-2, TIMP-4, PINP, PIIINP, CITP, cardiotrophin, sRAGE, osteopontin, and NTBNP. Thus, the method can comprise measuring, for example, MMP-2 and MMP-9, MMP-2 and MMP-3, MMP-2 and MMP-7, MMP-2 and MMP-13, MMP-2 and MMP-8, MMP-2 and TIMP-1, MMP-2 and TIMP-2, MMP-2 and TIMP-4, MMP-2 and PIIINP, MMP-2 and sRAGE, MMP-2 and cardiotrophin, MMP-2 and osteopontin, MMP-2 and NTBNP, MMP-2 and CITP, MMP-2 and PINP, MMP-9 and MMP-3, MMP-9 and MMP-7, MMP-9 and MMP-13, MMP-9 and MMP-8, MMP-9 and TIMP-1, MMP-9 and TIMP-2, MMP-9 and TIMP-4, MMP-9 and PIIINP, MMP-9 and sRAGE, MMP-9 and cardiotrophin, MMP-9 and osteopontin, MMP-9 and NTBNP, MMP-9 and CITP, MMP-9 and PINP, MMP-7 and MMP-3, MMP-7 and MMP-13, MMP-7 and MMP-8, MMP-7 and TIMP-1, MMP-7 and TIMP-2, MMP-7 and TIMP-4, MMP-7 and PIIINP, MMP-7 and sRAGE, MMP-7 and cardiotrophin, MMP-7 and osteopontin, MMP-7 and NTBNP, MMP-7 and CITP, MMP-7 and PINP, MMP-13 and MMP-3, MMP-13 and MMP-8, MMP-13 and TIMP-1, MMP-13 and TIMP-13, MMP-13 and TIMP-4, MMP-13 and PIIINP, MMP-13 and sRAGE, MMP-13 and cardiotrophin, MMP-13 and osteopontin, MMP-13 and NTBNP, MMP-13 and CITP, MMP-13 and PINP, MMP-8 and MMP-3, MMP-8 and TIMP-1, MMP-8 and TIMP-2, MMP-8 and TIMP-4, MMP-8 and PIIINP, MMP-8 and sRAGE, MMP-8 and cardiotrophin, MMP-8 and osteopontin, MMP-8 and NTBNP, MMP-8 and CITP, MMP-8 and PINP, TIMP-1 and MMP-3, TIMP-1 and TIMP-2, TIMP-1 and TIMP-4, TIMP-1 and PIIINP, TIMP-1 and sRAGE, TIMP-1 and cardiotrophin, TIMP-1 and osteopontin, TIMP-1 and NTBNP, TIMP-1 and CITP, TIMP-1 and PINP, TIMP-2 and MMP-3, TIMP-2 and TIMP-4, TIMP-2 and PIIINP, TIMP-2 and sRAGE, TIMP-2 and cardiotrophin, TIMP-2 and osteopontin, TIMP-2 and NTBNP, TIMP-2 and CITP, TIMP-2 and PINP, TIMP-4 and MMP-3, TIMP-4 and PIIINP, TIMP-4 and sRAGE, TIMP-4 and cardiotrophin, TIMP-4 and osteopontin, TIMP-4 and NTBNP, TIMP-4 and CITP, TIMP-4 and PINP, PIIINP and MMP-3, PIIINP and sRAGE, PIIINP and cardiotrophin, PIIINP and osteopontin, PIIINP and NTBNP, PIIINP and CITP, PIIINP and PINP, sRAGE and MMP-3, sRAGE and cardiotrophin, sRAGE and osteopontin, sRAGE and NTBNP, sRAGE and CITP, sRAGE and PINP, cardiotrophin and MMP-3, cardiotrophin and osteopontin, cardiotrophin and NTBNP, cardiotrophin and CITP, cardiotrophin and PINP, osteopontin and MMP-3, osteopontin and NTBNP, osteopontin and CITP, osteopontin and PINP, NTBNP and MMP-3, NTBNP and CITP, NTBNP and PINP, CITP and MMP-3, CITP and PINP, and PINP and MMP-3.

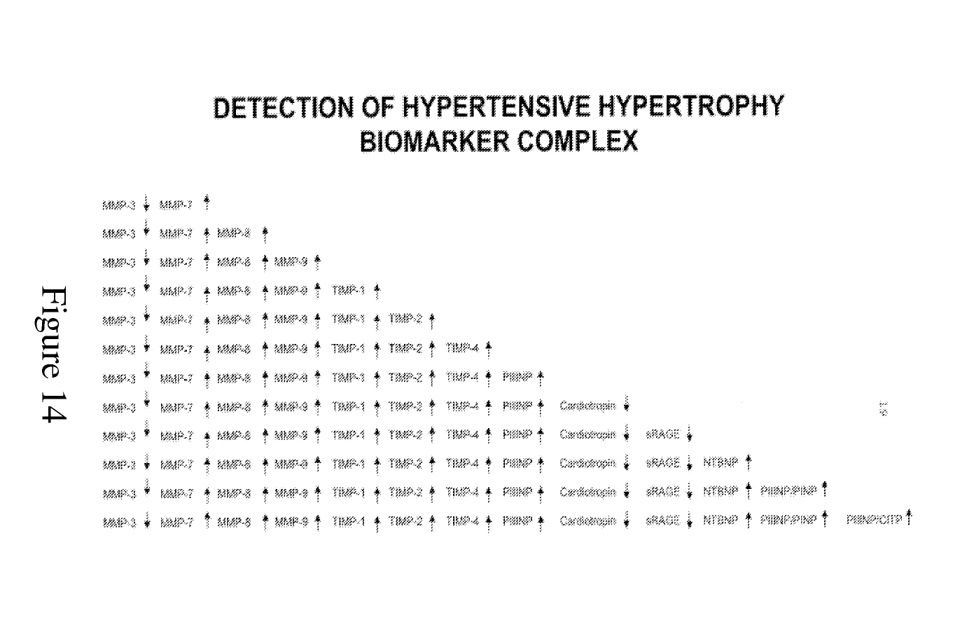

Thus, the method can comprise measuring MMP-2, MMP-13 and TIMP-1; MMP-2, MMP-13 and TIMP-2; MMP-2, MMP-13 and TIMP-4; MMP-13, TIMP-1, and TIMP-2; MMP-13, TIMP-1, and TIMP-4; MMP-13, TIMP-2, and TIMP-4. Thus, the method can comprise measuring MMP-2, MMP-13, TIMP-1, and TIMP-2; MMP-2, MMP-13, TIMP-1, and TIMP-4; MMP-2, MMP-13, TIMP-2, and TIMP-4; MMP-13, TIMP-1, TIMP-2, and TIMP-4; MMP-2, TIMP-1, TIMP-2, and TIMP-4. Thus, the method can comprise measuring MMP-2, MMP-13, TIMP-1, TIMP-2, and TIMP-4. Other combinations of these analytes are contemplated and disclosed herein. An example of a matrix of biomarkers to be used together is shown in FIG. 14.

### F. Rapid Screening for LVH

Provided is a rapid yes/no result that can be obtained by testing levels for one particular MMP, MMP-13. A set point, which can be adjusted based upon population statistics as well as age adjusted, would be used as the effective read-out. As an example, an MMP-13 level below a threshold setting of 1, 2, 3, 4, 5, 6, 7, 8, 9, or 10 ng/mL, would justify a more intensive plasma screening portfolio and additional cardiovascular imaging studies. In other words, this rapid screening test could be applied to any large population, which would then identify those subjects that would warrant more careful testing and follow-up. There are currently no available rapid screening tests to identify patients with LVH.

Provided is a method of predicting congestive heart failure and/or diastolic heart failure in a subject, comprising measuring the amount of MMP-13 in a body fluid from the subject, an amount of less than 1, 2, 3, 4, 5, 6, 7, 8, 9, or 10 ng/mL or undetectable indicating the presence of LVH and being predictive of CHF and/or DHF. When combined with abnormal measurements of other relevant analytes disclosed herein, this measurement can detect CHF and/or DHF.

Plasma profiling at a primary care or medical screening encounter can be performed. This screening measurement can be made for one or more of the biomarkers. If the one or more measurements falls outside reference values, additional measurements can be performed. For example, MMP-13 can be used for an initial screening such that if MMP-13 is non-detectable, then a second assay can be performed on the plasma sample. Likewise, MMP-9 and TIMP-1, TIMP-2, and/or TIMP-4 can be used for an initial screening such that if the ratio of MMP-9 to TIMP-1, TIMP-2, or TIMP-4 is less than normal limits using an established threshold, then a second assay can be performed on the plasma sample. This second test can be for the full profile shown in, for example, Table 3, Table 15, Table 16, Table 20, Table 21, Table 24, Table 25, FIG. 14, or a subset thereof. If this profile meets the criteria for hypertensive heart disease, then the patient can be evaluated by more aggressive tests which could include echocardiography, catheterization, nuclear imaging as appropriate. The patient can also be evaluated for more aggressive medical management.

### G. Diagnosis

Also provided is a diagnostic method that can be used, for example, with a subject that presents with signs and symptoms of CHF, but the underlying cause for this presentation is difficult to determine. This occurs quite frequently; where a patient has CHF, but whether LVH and DHF exists, and is contributory for the exacerbation of the CHF process, cannot be easily determined. The use of a simple and rapid blood test to “rule in” or “rule out” the presence of LVH and DHF, as described in this application, would provide this needed diagnostic approach. Specifically, a blood sample could be measured for, for example, MMP-13, MMP-9, MMP-2, TIMP-1, and/or TIMP-4. The obtained values would be compared to the normal reference values disclosed herein. If the values differ from the normal limits by the thresholds identified herein, then a patient can be identified to have DHF.

As other examples, the disclosed diagnostic method can be used with subjects who present no symptoms of hypertension or CHF, with subject with hypertension, with subjects with hypertension but no symptoms of CHF, with subjects with LVH, with subjects with LVH but no symptoms of CHF, with subjects with hypertension and LVH, with subjects with hypertension and LVH but no symptoms of CHF. This can serve to determine if the subject has or is at risk for CHF. Further, for subjects without diagnosed LVH, the disclosed diagnostic method can be used to determine if the subject has or is at risk for LVH.

For example, provided is a method of diagnosing LVH in a subject comprising measuring biomarker levels in a tissue or bodily fluid of the subject and comparing said levels to reference values.

In some aspects, MMP-2 plasma levels less than the normal value is an indication of hypertensive heart disease. For example, an amount of MMP-2 at least about 20% less than the normal mean value can be an indication of hypertensive heart disease. In some aspects, MMP-2 plasma levels less than about 1000 ng/ml, including less than about 1000, 990, 980, 970, 960, 950, 940, 930, 920, 920, 900, 890, 880, 870, 860, 850, 840, 830, 820, 810, 800, 790, 780, 770, 760, 750, 740, 730, 720, 710, 700, 650, 600, 550, 500, 450, 400, 350, 300, 250, 200, 250, or 100 ng/ml, is an indication of hypertensive heart disease.

In some aspects, MMP-9 plasma levels greater than the normal value is an indication of hypertensive heart disease. For example, an amount of MMP-9 at least about 50% greater than the normal mean value can be an indication of hypertensive heart disease. In some aspects, MMP-9 plasma levels greater than about 20 ng/ml, including greater than about 20, 21, 22, 23, 24, 15, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 ng/ml, is an indication of hypertensive heart disease.

In some aspects, undetectable MMP-13 plasma levels is an indication of LVH. In some aspects, MMP-13 plasma levels less than about 10 ng/ml, including less than about 10, 9, 8, 7, 6, 5, 4, 3, 2, or 1 ng/ml, is an indication of LVH.

In some aspects, TIMP-1 plasma levels greater than the normal value is an indication of hypertensive heart disease. For example, an amount of TIMP-1 at least about 50% greater than the normal mean value can be an indication of LVH. In some aspects, TIMP-1 plasma levels greater than about 1000 ng/ml, including greater than about 1000, 1010, 1020, 1030, 1040, 1050, 1060, 1070, 1080, 1090, 1100, 1150, 1200, 1250, 1300, 1350, 1400, or 1500 ng/ml, is an indication of LVH.

In some aspects, TIMP-2 plasma levels greater than the normal value is an indication of LVH. For example, an amount of TIMP-2 at least about 50% greater than the normal mean value can be an indication of LVH. In some aspects, TIMP-2 plasma levels greater than about 50 ng/ml, including greater than about 50, 55, 60, 65, 70, 75, 80, 85, 90, 95, or 100 ng/ml, is an indication of LVH.

In some aspects, TIMP-4 plasma levels greater than the normal value is an indication of LVH. For example, an amount of TIMP-4 at least about 50% greater than the normal mean value can be an indication of LVH. In some aspects, TIMP-4 plasma levels greater than about 2 ng/ml, including greater than about 2, 3, 4, 5, 6, 7, 8, 9, or 10 ng/ml, is an indication of LVH.

In some aspects, MMP-7 plasma levels within normal range is an indication of LVH. In some aspects, MMP-8 plasma levels within normal range is an indication of LVH.

In some aspects, MMP-3 plasma levels less than the normal value is an indication of LVH. For example, an amount of MMP-3 at least about 50% greater than the reference amount is an indication of LVH. In some aspects, MMP-3 plasma levels less than about 10 ng/ml, including less than about 10, 9.5, 9.0, 8.5, 8.0, 7.5, 7.0, and 6.5 ng/ml, is an indication of LVH.

In some aspects, PIIINP plasma levels greater than the normal value is an indication of LVH. For example, an amount of PIIINP at least about 10% greater than the reference amount is an indication of LVH. In some aspects, PIIINP plasma levels greater than about 8 ng/ml, including greater than about 8.0, 8.5, 9.0, 9.5, 10, 10.5, 11.0, 11.5, 12.0, and 12.5 ng/ml, is an indication of LVH.

In some aspects, CITP plasma levels greater than the normal value is an indication of LVH. For example, an amount of CITP at least about 20% greater than the reference amount is an indication of LVH. In some aspects, CITP plasma levels greater than about 2.2 ng/ml, including greater than about 2.2, 2.3, 2.4, 2.5, 2.6, 2.7, 2.8, 2.9, 3.0, 3.2, 3.4, 3.6, 3.8, and 4.0 ng/ml, is an indication of LVH.

In some aspects, NTBNP plasma levels greater than the normal value is an indication of LVH. For example, an amount of NTBNP at least about 50% greater than the reference amount is an indication of LVH. In some aspects, NTBNP plasma levels greater than about 150 ng/ml, including greater than about 150, 160, 170, 180, 190, 200, 220, 240, 260, 280, and 300 ng/ml, is an indication of LVH.

In some aspects, osteopontin plasma levels greater than the normal value is an indication of LVH. For example, an amount of osteopontin at least about 20% greater than the reference amount is an indication of LVH. In some aspects, osteopontin plasma levels greater than about 75 ng/ml, including greater than about 75, 80, 85, 90, 95, 100, 110, and 120 ng/ml, is an indication of LVH.

In some aspects, cardiotrophin plasma levels less than the normal value is an indication of LVH. For example, an amount of cardiotrophin at least about 30% less than the reference amount is an indication of LVH. In some aspects, cardiotrophin plasma levels less than about 20 ng/ml, including less than about 20, 19, 18, 17, 16, 15, 14, 13, 12, and 11 ng/ml, is an indication of LVH.

In some aspects, sRAGE plasma levels less than the normal value is an indication of LVH. For example, an amount of sRAGE at least about 10% less than the reference amount is an indication of LVH. In some aspects, sRAGE plasma levels less than about 2.6 ng/ml, including less than about 2.6, 2.5, 2.4, 2.3, 2.2, 2.1, 2.0, 1.8, 1.6, 1.4 and 1.2 ng/ml, is an indication of LVH.

As described more fully elsewhere herein, biomarker combinations, profiles, and matrices can be used in the disclosed methods. For example, the method can further comprise measuring plasma levels of two or more biomarkers. For example, the method can comprise measuring two, three, four, five, six, seven, eight, nine, ten, eleven, twelve, thirteen, fourteen, fifteen, or sixteen of MMP-2, MMP-3, MMP-9, MMP-7, MMP-13, MMP-8, TIMP-1, TIMP-2, TIMP-4, PINP, PIIINP, CITP, cardiotrophin, sRAGE, osteopontin, and NTBNP. Thus, the method can comprise measuring, for example, MMP-2 and MMP-9, MMP-2 and MMP-3, MMP-2 and MMP-7, MMP-2 and MMP-13, MMP-2 and MMP-8, MMP-2 and TIMP-1, MMP-2 and TIMP-2, MMP-2 and TIMP-4, MMP-2 and PIIINP, MMP-2 and sRAGE, MMP-2 and cardiotrophin, MMP-2 and osteopontin, MMP-2 and NTBNP, MMP-2 and CITP, MMP-2 and PINP, MMP-9 and MMP-3, MMP-9 and MMP-7, MMP-9 and MMP-13, MMP-9 and MMP-8, MMP-9 and TIMP-1, MMP-9 and TIMP-2, MMP-9 and TIMP-4, MMP-9 and PIIINP, MMP-9 and sRAGE, MMP-9 and cardiotrophin, MMP-9 and osteopontin, MMP-9 and NTBNP, MMP-9 and CITP, MMP-9 and PINP, MMP-7 and MMP-3, MMP-7 and MMP-13, MMP-7 and MMP-8, MMP-7 and TIMP-1, MMP-7 and TIMP-2, MMP-7 and TIMP-4, MMP-7 and PIIINP, MMP-7 and sRAGE, MMP-7 and cardiotrophin, MMP-7 and osteopontin, MMP-7 and NTBNP, MMP-7 and CITP, MMP-7 and PINP, MMP-13 and MMP-3, MMP-13 and MMP-8, MMP-13 and TIMP-1, MMP-13 and TIMP-13, MMP-13 and TIMP-4, MMP-13 and PIIINP, MMP-13 and sRAGE, MMP-13 and cardiotrophin, MMP-13 and osteopontin, MMP-13 and NTBNP, MMP-13 and CITP, MMP-13 and PINP, MMP-8 and MMP-3, MMP-8 and TIMP-1, MMP-8 and TIMP-2, MMP-8 and TIMP-4, MMP-8 and PIIINP, MMP-8 and sRAGE, MMP-8 and cardiotrophin, MMP-8 and osteopontin, MMP-8 and NTBNP, MMP-8 and CITP, MMP-8 and PINP, TIMP-1 and MMP-3, TIMP-1 and TIMP-2, TIMP-1 and TIMP-4, TIMP-1 and PIIINP, TIMP-1 and sRAGE, TIMP-1 and cardiotrophin, TIMP-1 and osteopontin, TIMP-1 and NTBNP, TIMP-1 and CITP, TIMP-1 and PINP, TIMP-2 and MMP-3, TIMP-2 and TIMP-4, TIMP-2 and PIIINP, TIMP-2 and sRAGE, TIMP-2 and cardiotrophin, TIMP-2 and osteopontin, TIMP-2 and NTBNP, TIMP-2 and CITP, TIMP-2 and PINP, TIMP-4 and MMP-3, TIMP-4 and PIIINP, TIMP-4 and sRAGE, TIMP-4 and cardiotrophin, TIMP-4 and osteopontin, TIMP-4 and NTBNP, TIMP-4 and CITP, TIMP-4 and PINP, PIIINP and MMP-3, PIIINP and sRAGE, PIIINP and cardiotrophin, PIIINP and osteopontin, PIIINP and NTBNP, PIIINP and CITP, PIIINP and PINP, sRAGE and MMP-3, sRAGE and cardiotrophin, sRAGE and osteopontin, sRAGE and NTBNP, sRAGE and CITP, sRAGE and PINP, cardiotrophin and MMP-3, cardiotrophin and osteopontin, cardiotrophin and NTBNP, cardiotrophin and CITP, cardiotrophin and PINP, osteopontin and MMP-3, osteopontin and NTBNP, osteopontin and CITP, osteopontin and PINP, NTBNP and MMP-3, NTBNP and CITP, NTBNP and PINP, CITP and MMP-3, CITP and PINP, and PINP and MMP-3. Thus, the method can comprise measuring MMP-2, MMP-13 and TIMP-1; MMP-2, MMP-13 and TIMP-2; MMP-2, MMP-13 and TIMP-4; MMP-13, TIMP-1, and TIMP-2; MMP-13, TIMP-1, and TIMP-4; MMP-13, TIMP-2, and TIMP-4. Thus, the method can comprise measuring MMP-2, MMP-13, TIMP-1, and TIMP-2; MMP-2, MMP-13, TIMP-1, and TIMP-4; MMP-2, MMP-13, TIMP-2, and TIMP-4; MMP-13, TIMP-1, TIMP-2, and TIMP-4; MMP-2, TIMP-1, TIMP-2, and TIMP-4. Thus, the method can comprise measuring MMP-2, MMP-13, TIMP-1, TIMP-2, and TIMP-4. Other combinations of these analytes are contemplated and disclosed herein.

For example, when combined with a reduced level of MMP-13, increased TIMP-1 (e.g., TIMP-1>1200 ng/mL) can detect CHF and/or DHF. As another example, when combined with a reduced level of MMP-13 and increased TIMP-1, an amount of TIMP-4 greater than 3 ng/mL indicates LVH and predicts CHF and/or DHF. Thus, a method of detecting LVH and predicting congestive heart failure and/or diastolic heart failure in a subject, comprises measuring in a body fluid from the subject the profiles of MMP-13, TIMP-1, and TIMP-4. The profiles wherein the amount of MMP-13 is undetectable, the amount of TIMP-1 is about 50% greater than normal value (or greater than 1200 ng/mL) and the amount of TIMP-4 is at least about 50% greater than normal value (or greater than 3 ng/mL) are predictive of CHF and/or DHF.

The method can further comprise calculating the ratio of one or more of the biomarkers to other biomarkers. For example, the method can comprise calculating the ratio of MMP-9 to TIMP-1, TIMP-2 or TIMP-4 or the ratio of PIIINP to PINP or CITP.

In some aspects, a ratio of MMP-9/TIMP-1 plasma levels less than the normal value is an indication of LVH. For example, a ratio of MMP-9/TIMP-1 at least about 50% less than the normal mean value can be an indication of LVH. For example, in some aspects, a ratio of MMP-9/TIMP-1 plasma levels less than about 7×10³, including less than about 7×10³, 6×10³, 5×10³, 4×10³, 5×10³, 6×10³, 1×10³, is an indication of LVH.

In some aspects, a ratio of MMP-9/TIMP-2 plasma levels less than the normal value is an indication of LVH. For example, a ratio of MMP-9/TIMP-2 at least about 50% less than the normal mean value can be an indication of LVH. In some aspects, a ratio of MMP-9/TIMP-2 plasma levels less than about 100×10³, including less than about 100×10³, 90×10³, 80×10³, 70×10³, 60×10³, 50×10³, 40×10³, 30×10³, 20×10³, or 10×10³, is an indication of LVH.

In some aspects, a ratio of MMP-9/TIMP-4 plasma levels less than the normal value is an indication of LVH. For example, a ratio of MMP-9/TIMP-4 at least about 50% less than the normal mean value can be an indication of LVH. In some aspects, a ratio of MMP-9/TIMP-4 plasma levels less than about 3, including less than about 3.0, 2.5, 2.0, 1.5, 1.0, 0.9, 0.8, 0.7, 0.6, 0.5, 0.4, 0.3, 0.2, 0.1, 0.05, or 0.01, is an indication of LVH.

In some aspects, a ratio of MMP-9/TIMP-1 plasma levels less than about 5×10³, a ratio of MMP-9/TIMP-2 plasma levels less than about 100×10³ and a ratio of MMP-9/TIMP-4 plasma levels less than about 1 is an indication of LVH.

In some aspects, MMP-2 plasma levels less than about 1000 ng/ml, MMP-13 plasma levels less than about 5 ng/ml, a ratio of MMP-9/TIMP-1 plasma levels less than about 5×10³ a ratio of MMP-9/TIMP-2 plasma levels less than about 100×10³ and a ratio of MMP-9/TIMP-4 plasma levels less than about 1 is an indication of LVH.

In some aspects, a ratio of PIIINP/PINP plasma levels of greater than the normal value is an indication of LVH. For example, a ratio of PIIINP/PINP greater than at least about 0.28 is an indication of LVH. In some aspects, a ratio of PIIINP/PINP greater than at least about 0.28, 0.29, 0.30, 0.31, 0.32, 0.34, 0.36, 0.38, 0.40, 0.5, 0.6, 0.8, and 1.0 is an indication of LVH.

In some aspects, a ratio of PIIINP/CITP plasma levels of greater than the normal value is an indication of LVH. For example, a ratio of PIIINP/CITP greater than at least about 5.5 is an indication of LVH. In some aspects, a ratio of PIIINP/PINP greater than at least about 5.5, 5.6, 5.8, 6.0, 6.2, 6.5, 7.0, 7.5, 8.0, and 9.0 is an indication of LVH.

### H. Prognosis

Also provided is a method of prognosis of congestive heart failure and/or diastolic heart failure that can be used, for example, with a subject who has been picked up on screening and then through a further plasma profile, is confirmed to have severe LVH and be at risk for developing CHF and/or DHF. In this case, for example, the MMP-13 level can be quantified as well as other biomarker levels. For example, a low/undetectable MMP-13 level (0-5 ng/mL) coupled with high TIMP levels (such as TIMP-1>1200 ng/mL, TIMP-2>700 ng/mL, and/or TIMP-4>3 ng/mL) in comparison to reference normal subjects coupled with TIMP levels will likely yield critical insight into the degree of myocardial fibrosis and diastolic dysfunction. This holds prognostic value as to the progression of symptoms and hospitalization. Specifically, these patients can be more aggressively treated with hypertensive medications, and have more regular cardiovascular imaging studies.

For example, provided is a method of identifying a subject at increased risk for developing congestive heart failure (CHF) and/or diastolic heart failure (DHF), comprising measuring biomarker levels in a tissue or bodily fluid of the subject and comparing said levels to reference values.

In some aspects, MMP-2 plasma levels less than the normal value is an indication of increased risk for developing congestive heart failure and/or diastolic heart failure. For example, an amount of MMP-2 at least about 20% less than the normal mean value can be an indication of increased risk for developing congestive heart failure and/or diastolic heart failure. In some aspects, MMP-2 plasma levels less than about 500 ng/ml, including less than about 500, 450, 400, 350, 300, 250, 200, 250, or 100 ng/ml, is an indication of increased risk for developing congestive heart failure and/or diastolic heart failure.

In some aspects, undetectable MMP-13 plasma levels is an indication of increased risk for developing congestive heart failure and/or diastolic heart failure. In some aspects, MMP-13 plasma levels less than about 10 ng/ml, including less than about 10, 9, 8, 7, 6, 5, 4, 3, 2, or 1 ng/ml, is an indication of increased risk for developing congestive heart failure and/or diastolic heart failure.

In some aspects, TIMP-1 plasma levels greater than the normal value is an indication of increased risk for developing congestive heart failure and/or diastolic heart failure. For example, an amount of TIMP-1 at least about 50% greater than the normal mean value can be an indication of increased risk for developing congestive heart failure and/or diastolic heart failure. In some aspects, TIMP-1 plasma levels greater than about 1000 ng/ml, including greater than about 1000, 1010, 1020, 1030, 1040, 1050, 1060, 1070, 1080, 1090, 1100, 1150, 1200, 1250, 1300, 1350, 1400, 1500, 1600, 1700, 1800, 1900, or 2000 ng/ml, is an indication of increased risk for developing congestive heart failure and/or diastolic heart failure.

In some aspects, TIMP-2 plasma levels greater than the normal value is an indication of increased risk for developing congestive heart failure and/or diastolic heart failure. For example, an amount of TIMP-2 at least about 50% greater than the normal mean value can be an indication of increased risk for developing congestive heart failure and/or diastolic heart failure. In some aspects, TIMP-2 plasma levels greater than about 50 ng/ml, including greater than about 50, 55, 60, 65, 70, 75, 80, 85, 90, 95, 100, 110, 120, 130, 140, 150, 160, 170, 180, 190, or 200 ng/ml, is an indication of increased risk for developing congestive heart failure and/or diastolic heart failure.

In some aspects, TIMP-4 plasma levels greater than the normal value is an indication of increased risk for developing congestive heart failure and/or diastolic heart failure. For example, an amount of TIMP-4 at least about 50% greater than the normal mean value can be an indication of increased risk for developing congestive heart failure and/or diastolic heart failure. In some aspects, TIMP-4 plasma levels greater than about 2 ng/ml, including greater than about 2, 3, 4, 5, 6, 7, 8, 9, 10, 15, 20, 25, 30, 35, 40, 45, or 50 ng/ml, is an indication of increased risk for developing congestive heart failure and/or diastolic heart failure.

In some aspects, MMP-9 plasma levels within normal range is an indication of increased risk for developing congestive heart failure and/or diastolic heart failure. In some aspects, MMP-7 plasma levels within normal range is an indication of increased risk for developing congestive heart failure and/or diastolic heart failure. In some aspects, MMP-8 plasma levels within normal range is an indication of increased risk for developing congestive heart failure and/or diastolic heart failure.

In some aspects, MMP-3 plasma levels less than the normal value is an indication of increased risk for developing congestive heart failure and/or diastolic heart failure. For example, an amount of MMP-3 at least about 50% greater than the reference amount is an indication of increased risk for developing congestive heart failure and/or diastolic heart failure. In some aspects, MMP-3 plasma levels less than about 10 ng/ml, including less than about 10, 9.5, 9.0, 8.5, 8.0, 7.5, 7.0, and 6.5 ng/ml, is an indication of increased risk for developing congestive heart failure and/or diastolic heart failure.

In some aspects, PIIINP plasma levels greater than the normal value is an indication of increased risk for developing congestive heart failure and/or diastolic heart failure. For example, an amount of PIIINP at least about 10% greater than the reference amount is an indication of increased risk for developing congestive heart failure and/or diastolic heart failure. In some aspects, PIIINP plasma levels greater than about 8 ng/ml, including greater than about 8.0, 8.5, 9.0, 9.5, 10, 10.5, 11.0, 11.5, 12.0, and 12.5 ng/ml, is an indication of increased risk for developing congestive heart failure and/or diastolic heart failure.

In some aspects, CITP plasma levels greater than the normal value is an indication of increased risk for developing congestive heart failure and/or diastolic heart failure. For example, an amount of CITP at least about 20% greater than the reference amount is an indication of increased risk for developing congestive heart failure and/or diastolic heart failure. In some aspects, CITP plasma levels greater than about 2.2 ng/ml, including greater than about 2.2, 2.3, 2.4, 2.5, 2.6, 2.7, 2.8, 2.9, 3.0, 3.2, 3.4, 3.6, 3.8, and 4.0 ng/ml, is an indication of increased risk for developing congestive heart failure and/or diastolic heart failure.

In some aspects, NTBNP plasma levels greater than the normal value is an indication of increased risk for developing congestive heart failure and/or diastolic heart failure. For example, an amount of NTBNP at least about 50% greater than the reference amount is an indication of increased risk for developing congestive heart failure and/or diastolic heart failure. In some aspects, NTBNP plasma levels greater than about 150 ng/ml, including greater than about 150, 160, 170, 180, 190, 200, 220, 240, 260, 280, and 300 ng/ml, is an indication of increased risk for developing congestive heart failure and/or diastolic heart failure.

In some aspects, osteopontin plasma levels greater than the normal value is an indication of increased risk for developing congestive heart failure and/or diastolic heart failure. For example, an amount of osteopontin at least about 20% greater than the reference amount is an indication of increased risk for developing congestive heart failure and/or diastolic heart failure. In some aspects, osteopontin plasma levels greater than about 75 ng/ml, including greater than about 75, 80, 85, 90, 95, 100, 110, and 120 ng/ml, is an indication of increased risk for developing congestive heart failure and/or diastolic heart failure.

In some aspects, cardiotrophin plasma levels less than the normal value is an indication of increased risk for developing congestive heart failure and/or diastolic heart failure. For example, an amount of cardiotrophin at least about 30% less than the reference amount is an indication of increased risk for developing congestive heart failure and/or diastolic heart failure. In some aspects, cardiotrophin plasma levels less than about 20 ng/ml, including less than about 20, 19, 18, 17, 16, 15, 14, 13, 12, and 11 ng/ml, is an indication of increased risk for developing congestive heart failure and/or diastolic heart failure.

In some aspects, sRAGE plasma levels less than the normal value is an indication of increased risk for developing congestive heart failure and/or diastolic heart failure. For example, an amount of sRAGE at least about 10% less than the reference amount is an indication of increased risk for developing congestive heart failure and/or diastolic heart failure. In some aspects, sRAGE plasma levels less than about 2.6 ng/ml, including less than about 2.6, 2.5, 2.4, 2.3, 2.2, 2.1, 2.0, 1.8, 1.6, 1.4 and 1.2 ng/ml, is an indication of increased risk for developing congestive heart failure and/or diastolic heart failure.

As described more fully elsewhere herein, biomarker combinations, profiles, and matrices can be used in the disclosed methods. For example, the method can further comprise measuring plasma levels of two or more biomarkers. For example, the method can comprise measuring two, three, four, five, six, seven, eight, nine, ten, eleven, twelve, thirteen, fourteen, fifteen, or sixteen of MMP-2, MMP-3, MMP-9, MMP-7, MMP-13, MMP-8, TIMP-1, TIMP-2, TIMP-4, PINP, PIIINP, CITP, cardiotrophin, sRAGE, osteopontin, and NTBNP. Thus, the method can comprise measuring, for example, MMP-2 and MMP-9, MMP-2 and MMP-3, MMP-2 and MMP-7, MMP-2 and MMP-13, MMP-2 and MMP-8, MMP-2 and TIMP-1, MMP-2 and TIMP-2, MMP-2 and TIMP-4, MMP-2 and PIIINP, MMP-2 and sRAGE, MMP-2 and cardiotrophin, MMP-2 and osteopontin, MMP-2 and NTBNP, MMP-2 and CITP, MMP-2 and PINP, MMP-9 and MMP-3, MMP-9 and MMP-7, MMP-9 and MMP-13, MMP-9 and MMP-8, MMP-9 and TIMP-1, MMP-9 and TIMP-2, MMP-9 and TIMP-4, MMP-9 and PIIINP, MMP-9 and sRAGE, MMP-9 and cardiotrophin, MMP-9 and osteopontin, MMP-9 and NTBNP, MMP-9 and CITP, MMP-9 and PINP, MMP-7 and MMP-3, MMP-7 and MMP-13, MMP-7 and MMP-8, MMP-7 and TIMP-1, MMP-7 and TIMP-2, MMP-7 and TIMP-4, MMP-7 and PIIINP, MMP-7 and sRAGE, MMP-7 and cardiotrophin, MMP-7 and osteopontin, MMP-7 and NTBNP, MMP-7 and CITP, MMP-7 and PINP, MMP-13 and MMP-3, MMP-13 and MMP-8, MMP-13 and TIMP-1, MMP-13 and TIMP-13, MMP-13 and TIMP-4, MMP-13 and PIIINP, MMP-13 and sRAGE, MMP-13 and cardiotrophin, MMP-13 and osteopontin, MMP-13 and NTBNP, MMP-13 and CITP, MMP-13 and PINP, MMP-8 and MMP-3, MMP-8 and TIMP-1, MMP-8 and TIMP-2, MMP-8 and TIMP-4, MMP-8 and PIIINP, MMP-8 and sRAGE, MMP-8 and cardiotrophin, MMP-8 and osteopontin, MMP-8 and NTBNP, MMP-8 and CITP, MMP-8 and PINP, TIMP-1 and MMP-3, TIMP-1 and TIMP-2, TIMP-1 and TIMP-4, TIMP-1 and PIIINP, TIMP-1 and sRAGE, TIMP-1 and cardiotrophin, TIMP-1 and osteopontin, TIMP-1 and NTBNP, TIMP-1 and CITP, TIMP-1 and PINP, TIMP-2 and MMP-3, TIMP-2 and TIMP-4, TIMP-2 and PIIINP, TIMP-2 and sRAGE, TIMP-2 and cardiotrophin, TIMP-2 and osteopontin, TIMP-2 and NTBNP, TIMP-2 and CITP, TIMP-2 and PINP, TIMP-4 and MMP-3, TIMP-4 and PIIINP, TIMP-4 and sRAGE, TIMP-4 and cardiotrophin, TIMP-4 and osteopontin, TIMP-4 and NTBNP, TIMP-4 and CITP, TIMP-4 and PINP, PIIINP and MMP-3, PIIINP and sRAGE, PIIINP and cardiotrophin, PIIINP and osteopontin, PIIINP and NTBNP, PIIINP and CITP, PIIINP and PINP, sRAGE and MMP-3, sRAGE and cardiotrophin, sRAGE and osteopontin, sRAGE and NTBNP, sRAGE and CITP, sRAGE and PINP, cardiotrophin and MMP-3, cardiotrophin and osteopontin, cardiotrophin and NTBNP, cardiotrophin and CITP, cardiotrophin and PINP, osteopontin and MMP-3, osteopontin and NTBNP, osteopontin and CITP, osteopontin and PINP, NTBNP and MMP-3, NTBNP and CITP, NTBNP and PINP, CITP and MMP-3, CITP and PINP, and PINP and MMP-3. Thus, the method can comprise measuring MMP-2, MMP-13 and TIMP-1; MMP-2, MMP-13 and TIMP-2; MMP-2, MMP-13 and TIMP-4; MMP-13, TIMP-1, and TIMP-2; MMP-13, TIMP-1, and TIMP-4; MMP-13, TIMP-2, and TIMP-4. Thus, the method can comprise measuring MMP-2, MMP-13, TIMP-1, and TIMP-2; MMP-2, MMP-13, TIMP-1, and TIMP-4; MMP-2, MMP-13, TIMP-2, and TIMP-4; MMP-13, TIMP-1, TIMP-2, and TIMP-4; MMP-2, TIMP-1, TIMP-2, and TIMP-4. Thus, the method can comprise measuring MMP-2, MMP-13, TIMP-1, TIMP-2, and TIMP-4. Other combinations of these analytes are contemplated and disclosed herein.

For example, provided is a method of detecting congestive heart failure and/or diastolic heart failure in a subject, comprising measuring in a body fluid from the subject an amount of MMP-13, TIMP-1, TIMP-4 and MMP-9. Also provided is a method of predicting congestive heart failure and/or diastolic heart failure in a subject, comprising measuring in a body fluid from the subject an amount of MMP-13, TIMP-1, TIMP-4 and MMP-9. In these methods, the profiles can show an amount of MMP-13 that is undetectable (or less than 10 ng/mL), an amount of TIMP-1 that is about 50% greater than normal value or greater than 1200 ng/mL, an amount of TIMP-4 that is at least about 50% greater than normal value or greater than 3 ng/mL and an amount of MMP-9 that is at least about 50% greater than normal value can detect LVH, CHF and/or DHF.

Also provided is a method of detecting congestive heart failure and/or diastolic heart failure in a subject, comprising measuring in a body fluid from the subject an amount of MMP-13, TIMP-1, TIMP-4 and MMP-2. Also provided is a method of predicting congestive heart failure and/or diastolic heart failure in a subject, comprising measuring in a body fluid from the subject an amount of MMP-13, TIMP-1, TIMP-4 and MMP-2. In these methods, the profiles can show an amount of MMP-13 that is undetectable (or less than 10 ng/mL), an amount of TIMP-1 that is about 50% greater than normal value (or greater than 1200 ng/mL), an amount of TIMP-4 that is at least about 50% greater than normal value (or greater than 3 ng/mL) and the amount of MMP-2 is at least about 20% less than normal value (or less than 1200 ng/mL).

The method can further comprise calculating the ratio of one or more of the biomarkers to other biomarkers. For example, the method can comprise calculating the ratio of MMP-9 to TIMP-1, TIMP-2 or TIMP-4 or the ratio of PIIINP to PINP or CITP.

In some aspects, a ratio of MMP-9/TIMP-1 plasma levels less than the normal value is an indication of LVH. For example, a ratio of MMP-9/TIMP-1 at least about 50% less than the normal mean value can be an indication of increased risk for developing congestive heart failure and/or diastolic heart failure. For example, in some aspects, a ratio of MMP-9/TIMP-1 plasma levels less than about 7×10³, including less than about 7×10³, 6×10³, 5×10³, 4×10³, 5×10³, 6×10³, 1×10³, 9×10², 8×10², 7×10², 6×10², 5×10², 4×10², 3×10², 2×10², or 1×10², is an indication of increased risk for developing congestive heart failure and/or diastolic heart failure.

In some aspects, a ratio of MMP-9/TIMP-2 plasma levels less than the normal value is an indication of LVH. For example, a ratio of MMP-9/TIMP-2 at least about 50% less than the normal mean value can be an indication of LVH. In some aspects, a ratio of MMP-9/TIMP-2 plasma levels less than about 100×10³, including less than about 100×10³, 90×10³, 80×10³, 70×10³, 60×10³, 50×10³, 40×10³, 30×10³, 20×10³, 10×10³, 9×10³, 8×10³, 7×10³, 6×10³, 5×10³, 4×10³, 3×10³, 2×10³, or 1×10³, is an indication of LVH.

In some aspects, a ratio of MMP-9/TIMP-4 plasma levels less than the normal value is an indication of LVH. For example, a ratio of MMP-9/TIMP-4 at least about 100% less than the normal mean value can be an indication of LVH. In some aspects, a ratio of MMP-9/TIMP-4 plasma levels less than about 1, including less than about 1, 0.9, 0.8, 0.7, 0.6, 0.5, 0.4, 0.3, 0.2, 0.25, 0.2, 0.15, 0.10, 0.05, or 0.01, is an indication of LVH.

Thus, provided is a method of detecting or predicting congestive heart failure and/or diastolic heart failure in a subject, comprising detecting a reduction in the ratio of MMP-9 to TIMP-4 in a body fluid from the subject compared to the normal ratio is provided. The method involves measuring a reduction in the ratio of at least about 50% compared to the normal ratio.

In some aspects, a ratio of MMP-9/TIMP-1 plasma levels less than about 5×10³, a ratio of MMP-9/TIMP-2 plasma levels less than about 100×10³ and a ratio of MMP-9/TIMP-4 plasma levels less than about 1 is an indication of LVH.

In some aspects, MMP-2 plasma levels less than about 1000 ng/ml, MMP-13 plasma levels less than about 5 ng/ml, a ratio of MMP-9/TIMP-1 plasma levels less than about 5×10³ a ratio of MMP-9/TIMP-2 plasma levels less than about 100×10³ and a ratio of MMP-9/TIMP-4 plasma levels less than about 1 is an indication of LVH.

In some aspects, a ratio of PIIINP/PINP plasma levels of greater than the normal value is an indication of LVH. For example, a ratio of PIIINP/PINP greater than at least about 0.28 is an indication of LVH. In some aspects, a ratio of PIIINP/PINP greater than at least about 0.28, 0.29, 0.30, 0.31, 0.32, 0.34, 0.36, 0.38, 0.40, 0.5, 0.6, 0.8, and 1.0 is an indication of LVH.

In some aspects, a ratio of PIIINP/CITP plasma levels of greater than the normal value is an indication of LVH. For example, a ratio of PIIINP/CITP greater than at least about 5.5 is an indication of LVH. In some aspects, a ratio of PIIINP/PINP greater than at least about 5.5, 5.6, 5.8, 6.0, 6.2, 6.5, 7.0, 7.5, 8.0, and 9.0 is an indication of LVH.

### I. Guiding Therapeutic Interventions

With respect to treatment, biomarker levels, such as low MMP-13 and high TIMP levels, can be monitored as an indicator of pharmacological efficacy. Thus, disclosed are methods comprising measuring the amount of two or more biomarkers in a body fluid from a subject, wherein the subject has hypertension, wherein measuring the amount of the two or more biomarkers in a body fluid from a subject is performed in order to monitor the effect or effectiveness of a treatment of the subject for hypertension, left ventricular hypertrophy, congestive heart failure, or a combination.

Also disclosed are methods comprising measuring the amount of two or more biomarkers in a body fluid from a subject, wherein measuring the amount of the two or more biomarkers in a body fluid from a subject is performed in order to monitor the subject for the development of left ventricular hypertrophy, congestive heart failure, or both.

There are several relevant clinical scenarios for which this would be highly applicable. For example, while a hypertensive patient may have blood pressure within “normal limits”, MMP-13 remains suppressed and TIMP levels are increased. Up titration of certain hypertension medications could then be utilized to “normalize” these biological markers of myocardial fibrosis, congestive heart failure and diastolic heart failure. This approach is to serially measure blood values of biomarkers (for example, the MMPs and TIMPs shown in Table 3), and to increase medication in order to bring these profiles to within the normal reference range.

In hypertensive patients that have been identified to have increased heart mass (size) due to high blood pressure, biomarker profiles can be utilized to follow the adequacy of treatment. The specific profiles identified as disclosed herein can be monitored and efficacy of treatment determined as these biomarker profiles moved towards the normal range.

The biomarker profiles are based on measurements of individual biomarkers. The amounts of these can be measured by any means known to provide an acceptable indication of how much of any of these is present in the sample being analyzed. An example of a means of measuring is provided in the Examples. The process of measuring an amount of an analyte (e.g., MMPs, TIMPs, PINP, PIIINP, CITP, cardiotrophin, sRAGE, osteopontin) includes a measurement of no amount or an undetectable amount of the analyte. The techniques and approaches for measuring biomarkers can be based upon high sensitivity immunoassays. Several of these immunoassays were developed (e.g., TIMP-4 assay measurements).

The immunoassay approach which was standardized for providing the measurements shown in Table 1 were performed by an enzyme linked immuno-assay (ELISA). However, other more sensitive and rapid methods for measuring blood levels of biomarkers have been performed and these include the use of a multiplex assay system. In this example, multiple analytes in volume-limited samples, such as plasma or other biological samples, can be measured using a bead-based multiplex sandwich immunoassay. This emergent technique for multiplex analysis is built on technology that combines the sensitivity of ELISA with flow cytometric detection, allowing for the specific measurement of up to 100 different analytes within a single sample of less than 50 μl. This approach will allow for the measurement of multiple biomarkers in a small blood sample. This type of approach can be used for the diagnostic, prognostic, predictive and therapeutic monitoring applications that are described herein. Specifically, to measure analyte concentrations simultaneously, the microbeads are incubated with sample (i.e. blood sample) and allowed to form complexes with the specific analytes of interest (e.g., MMPs, TIMPs, PINP, PIIINP, CITP, cardiotrophin, sRAGE, osteopontin). Detection antibodies (biotinylated), specific for a second epitope on each analyte, are then added to the mixture and allowed to bind to the microbeads complexed with analyte. The mixture is then incubated with a fluorescent reporter molecule (streptavidin-phycoerythrin) and the entire sample is passed through a two-laser flow cytometric detector. One laser detects the precise fluorescence of the microbead which defines the specific analyte being examined, and the other laser detects the amount of reporter fluorescence which is directly proportional to the amount of analyte bound. This process has been applied to a number of biomarkers that are relevant to the CHF process and some of these are shown in FIG. 8, and Table 1. This is but one example of how single or multiple analytes can be measured with a very small blood sample. Other examples of measurements that have been performed with respect to biomarkers include radioimmunoassay and immunoblotting assays. These approaches are also antibody based. These and other biomarkers and biomarker measurements are shown in Tables 3, 15, 16, 20, 21, 24, 25, and FIG. 14.

### J. Monitoring Development of Heart Disease

Also disclosed are methods of monitoring the development of heart disease. For example, disclosed are methods of monitoring the transition from hypertension without LVH to LVH and identifying subjects with hypertension who are developing or are at risk of developing LVH. Also disclosed are methods of monitoring the transition from hypertension and/or LVH to CHF and/or DHF and identifying subjects with hypertension and/or LVH who are developing or are at risk of developing CHF and/or DHF.

Accordingly, disclosed is a method of, for example, monitoring the transition from hypertension without LVH to LVH and identifying subjects with hypertension who are developing or are at risk of developing LVH, comprising measuring the amount of two or more biomarkers in a body fluid from a subject, wherein the subject has hypertension, wherein the amount of two or more of the two or more biomarkers compared to a reference amount (for example, the amount in a normal subject, the amount in a subject with hypertension, or the amount in a subject with left ventricular hypertrophy) for the biomarker indicates the development or risk of development of left ventricular hypertrophy in the subject.

The amount of osteopontin in a body fluid from the subject can be measured, wherein the reference amount can be the amount in a subject with hypertension, wherein an amount of osteopontin that is greater than the reference amount indicates the development or risk of development of left ventricular hypertrophy in the subject. The amount of osteopontin can be at least about 20% greater than the reference amount.

The amount of cardiotrophin in a body fluid from the subject can be measured, wherein the reference amount can be the amount in a subject with hypertension, wherein an amount of cardiotrophin that is less than the reference amount indicates the development or risk of development of left ventricular hypertrophy in the subject. The amount of cardiotrophin can be at least about 30% less than the reference amount.

The amount of osteopontin and cardiotropin in a body fluid from the subject can be measured. The amount of osteopontin can be at least about 20% greater than the reference amount, and the amount of cardiotrophin can be at least about 30% less than the reference amount.

Also disclosed is a method of, for example, monitoring the transition from hypertension and/or LVH to CHF and/or DHF and identifying subjects with hypertension and/or LVH who are developing or are at risk of developing CHF and/or DHF, comprising measuring the amount of two or more biomarkers in a body fluid from a subject, wherein the subject has hypertension, wherein the amount of two or more of the two or more biomarkers compared to a reference amount (for example, the amount in a normal subject, the amount in a subject with hypertension, or the amount in a subject with left ventricular hypertrophy) for the biomarker indicates the development or risk of development of congestive heart failure in the subject.

Also disclosed is a method of, for example, monitoring the transition from hypertension and/or LVH to CHF and/or DHF and identifying subjects with hypertension and/or LVH who are developing or are at risk of developing CHF and/or DHF, comprising measuring the amount of two or more biomarkers in a body fluid from a subject, wherein the subject has left ventricular hypertrophy, wherein the amount of two or more of the two or more biomarkers compared to a reference amount (for example, the amount in a normal subject, the amount in a subject with hypertension, or the amount in a subject with left ventricular hypertrophy) for the biomarker indicates the development or risk of development of congestive heart failure in the subject.

The amount of MMP-2 in a body fluid from the subject can be measured, wherein the reference amount can be the amount in a subject with left ventricular hypertrophy, wherein an amount of MMP-2 that is greater than the reference amount indicates the development or risk of development of congestive heart failure in the subject. The amount of MMP-2 can be at least about 10% greater than the reference amount.

The amount of MMP-7 in a body fluid from the subject can be measured, wherein the reference amount can be the amount in a normal subject, wherein an amount of MMP-7 that is greater than the reference amount indicates the development or risk of development of congestive heart failure in the subject. The amount of MMP-7 can be at least about 30% greater than the reference amount.

The amount of TIMP-2 in a body fluid from the subject can be measured, wherein the reference amount can be the amount in a subject with left ventricular hypertrophy, wherein an amount of TIMP-2 that is less than the reference amount indicates the development or risk of development of congestive heart failure in the subject. The amount of TIMP-2 can be at least about 10% less than the reference amount.

The amount of sRAGE in a body fluid from the subject can be measured, wherein the reference amount can be the amount in a subject with left ventricular hypertrophy, wherein an amount of sRAGE that is greater than the reference amount indicates the development or risk of development of congestive heart failure in the subject. The amount of sRAGE can be at least about 10% greater than the reference amount.

The amount of CITP in a body fluid from the subject can be measured, wherein the reference amount can be the amount in a normal subject, wherein an amount of CITP that is greater than the reference amount indicates the development or risk of development of congestive heart failure in the subject. The amount of CITP can be at least about 20% greater than the reference amount.

The amount of PIIINP and CITP in a body fluid from the subject can be measured, wherein an increase in the ratio of PIIINP to CITP compared to a reference ratio indicates the presence or risk of congestive heart failure in the subject, wherein the reference ratio can be the ratio in a normal subject. The reduction in the ratio can be at least about 10% compared to the reference ratio.

The amount of PIIINP and PINP in a body fluid from the subject can be measured, wherein an increase in the ratio of PIIINP to PINP compared to a reference ratio indicates the presence or risk of congestive heart failure in the subject, wherein the reference ratio can be the ratio in a normal subject. The reduction in the ratio can be at least about 20% compared to the reference ratio.

### K. Combination

The herein disclosed methods can further comprise detecting other markers of heart failure. For example, the herein disclosed methods can further comprise measuring Troponin-I levels in a tissue or bodily fluid of the subject and comparing said levels to reference values.

### L. Timing of Measurements

As described below and elucidated in further examples for screening and therapeutic monitoring, the timing of measurements would be context specific. For screening, this can be anytime a subject is presenting for a medical examination. Examples of this would include annual physicals, health fairs, and screening through residential facilities. Thus, the disclosed diagnostic method can be used to diagnose a subject that presents with signs and symptoms of CHF, but the underlying cause for this presentation is difficult to determine

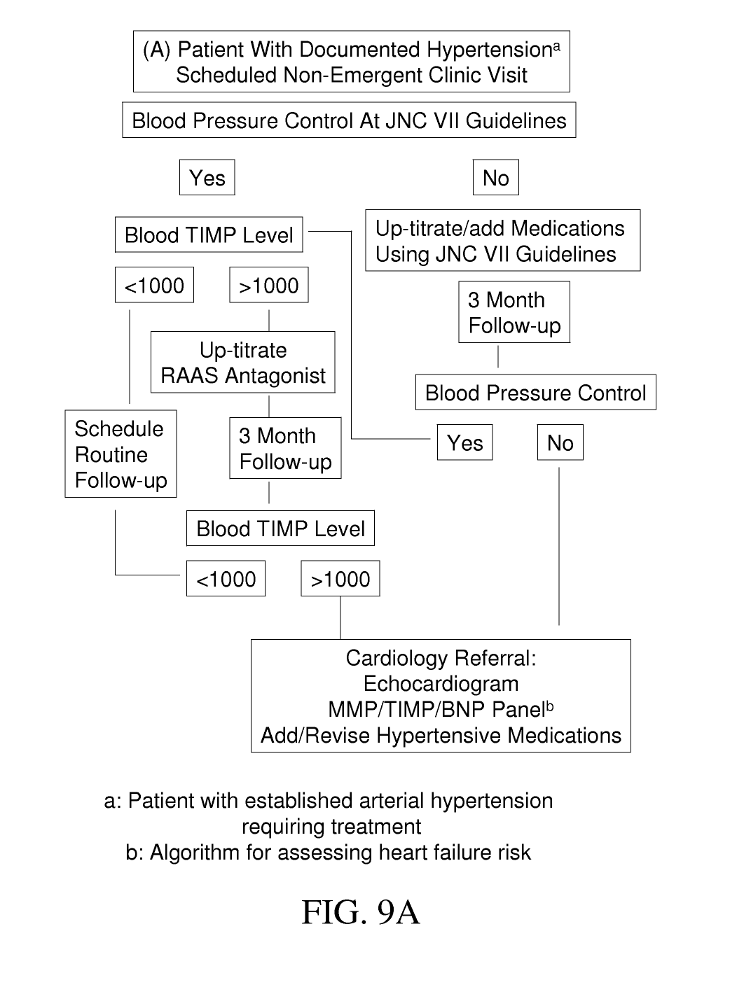

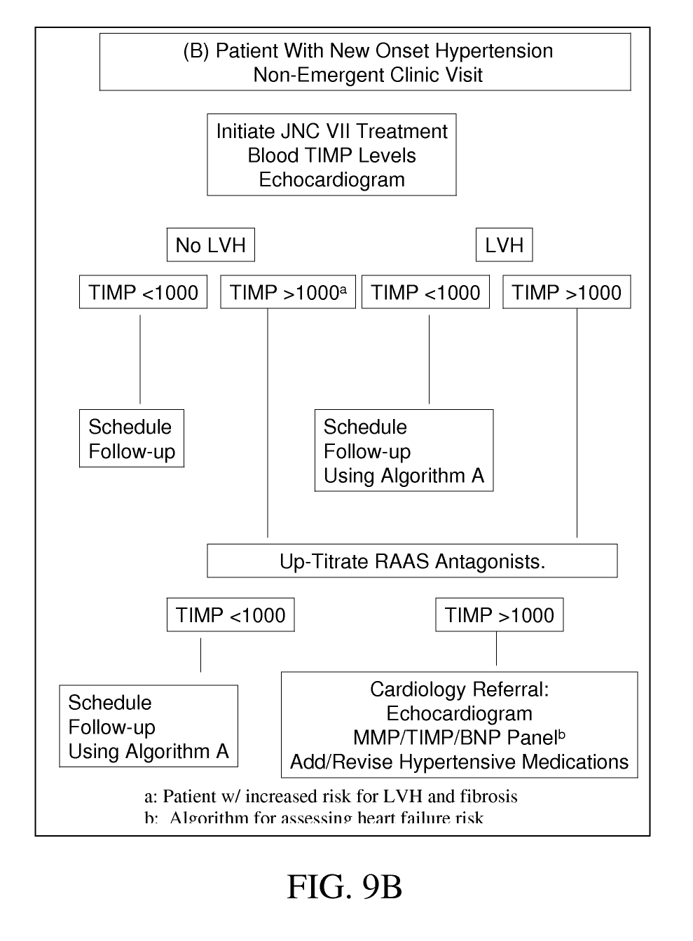

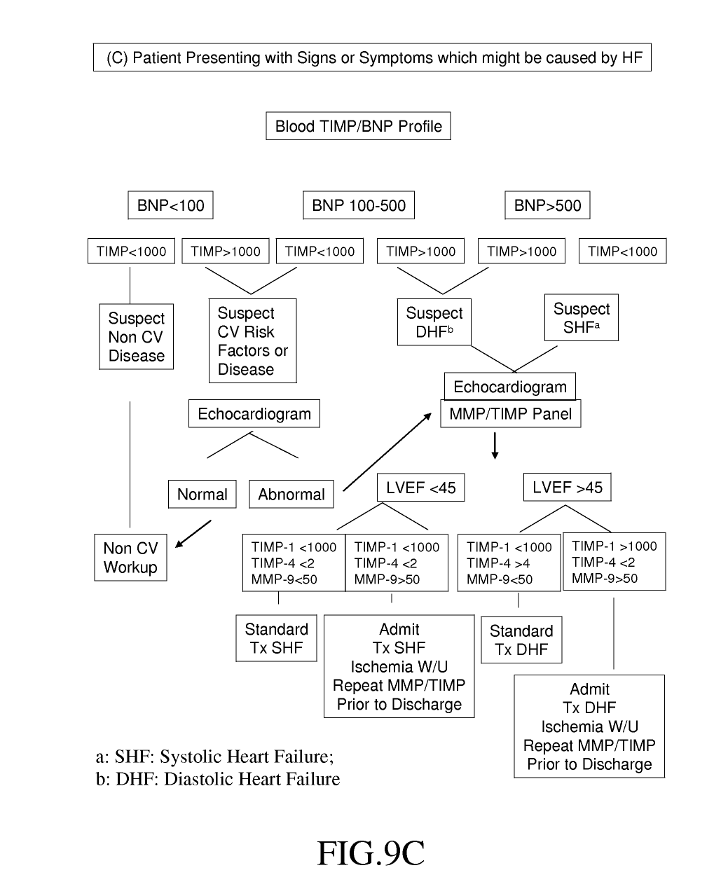

There are at least three initial time points for biomarker profiling for the methods disclosed herein. Initial measurements can be taken in a patient presenting for a routine clinic visit with history of established hypertension. Initial measurements can be taken at a health fair which would precipitate a clinic visit. Initial measurements can be taken in a patient presenting with symptoms due to hypertensive heart failure. The schematics in how the sampling and diagnostic approach for each of these scenarios is shown in FIGS. 9A-C for each of these cases.

Thus, the disclosed method of prognosis can be used to identify whether a subject that presents with high blood pressure (hypertension) has LVH or is at risk for developing DHF. The disclosed method of prognosis can also be used to identify whether a subject that presents with signs and symptoms of CHF has LVH and is at risk for developing of congestive heart failure (CHF) and/or diastolic heart failure (DHF). For example, the method can be used with a patient that presents to the physician with complaints consistent with CHF. The physician can then apply the blood tests to determine whether a biomarker profile consistent with LVH, CHF and/or DHF is present. This would guide the physician into further diagnostic testing and treatment plans.

Another example of timing of blood sampling would be when a patient has been identified to have established LVH, then serially monitoring biomarker profiles could be used as predictive tools for the progression of CHF and/or DHF. These tests could be applied only once as a screening tool, or applied multiple times and sequentially in any given subject.

### M. Kits

Disclosed herein are kits that are drawn to reagents that can be used in practicing the methods disclosed herein. The kits can include any reagent or combination of reagents discussed herein or that would be understood to be required or beneficial in the practice of the disclosed methods. For example, disclosed is a kit for assessing a subject's risk for developing DHF, in which components include components described in the previous section. For example, the components of a biomarker kit would include the necessary reagents for complexing to the relevant biomarker of interest (See Tables 3, 15, 16, 20, 21, 24, 25, and FIG. 14 for lists of relevant biomarkers) to a detection reagent. In the example of an immunoassay approach, a fluorescently labeled antibody against a specific biomarker would be incubated with the blood sample and following a washing and non-specific binding clearance step, the amount of antibody bound to the biomarker of interest would be computed by measuring the relative degree of fluorescence. This can be a very simple kit which could be used for screening, or a more complex system where multiple biomarkers are measured from a single sample. A rationale for a graduated approach for measuring one biomarker of interest to measuring multiple biomarkers simultaneously has been described elsewhere herein. For a screening assay (e.g., for MMP-13) the small blood sample would be processed into plasma (centrifugation) and the plasma mixed with the MMP-13 targeted antibody. The mixture would be centrifuged again, and the specifically bound antibody bound to MMP-13 would be read by a fluorimetry system. This equipment and measurement system could be easily fashioned into a small suitcase or table top system. The readout from the system would then indicate whether MMP-13 is below or above a specific threshold measurement (as defined in a previous section).

### EXAMPLES

### A. Example 1

### Matrix Metalloproteinases/Tissue Inhibitors of Metalloproteinases: Relationship Between Changes in Proteolytic Determinants of Matrix Compostition and Structural, Functional and Clinical Manifestations of Hypertensive Heart Disease

Summary of Methods and Results: Plasma MMP-2, -9, -13, and TIMP-1, -2 and Doppler echocardiography were obtained in 103 subjects divided into 4 groups: a) reference subjects (CTL) with no evidence of cardiovascular disease, b) hypertension (HTN), controlled blood pressure, and no LV hypertrophy, c) hypertension, controlled blood pressure, with LV hypertrophy (HTN&LVH), but no CHF, d) hypertension, controlled blood pressure, LVH, and CHF (HTN&LVH&CHF). Compared with CTL, patients with HTN had no significant changes in any MMP or TIMP. Patients with HTN&LVH had decreased MMP-2 and MMP-13, and increased MMP-9. Only patients with HTN&LVH&CHF had increased TIMP-1. TIMP-1>1200 ng/mL was predictive of CHF.

Conclusion: Patients with hypertension but normal LV structure and function had normal MMP/TIMP profiles. Changes in MMP profiles which favor decreased ECM degradation were associated with LV hypertrophy and diastolic dysfunction. Increased TIMP-1 predicted the presence of CHF. These data indicate that changes in MMP/TIMP balance play an important role in the structural, functional and clinical manifestations of hypertensive heart disease.

1. Methods

Subjects: Two groups of subjects were recruited into this study: reference controls and patients with LVH. Reference controls were identified from locally sponsored health fairs and volunteers from the Medical University of South Carolina staff. Of the reference controls screened, 35% were enrolled, 50% had one of the exclusion criteria listed below and 15% declined participation. LVH patients were identified from echocardiographic studies. Of the patient echocardiograms screened, 10% were enrolled, 75% had one of the exclusion criteria listed below and 15% declined participation. There were some exclusion criteria common to both groups:

1) history of myocardial infarction, 2) regional wall motion abnormality, 3) coronary revascularization surgery, 4) amyloidosis, sarcoidosis, HIV, hypertrophic obstructive cardiomyopathy, valvular heart disease, 5) ejection fraction<50%, 6) malignancy, 7) significant renal or hepatic dysfunction, 8) rheumatological disease, 9) blood pressure>140/90 mmHg.

One hundred and three subjects were enrolled in this study: 53 reference control subjects and 50 subjects with evidence of LVH [LV wall thickness of >1.2 cm and/or LV mass index≧125 gm/m² (Table 4)]. The reference control subjects were subdivided into two groups based on the presence or absence of hypertension; 39 control subjects (referred to as “Reference control without hypertension”), had no history of hypertension, no evidence of cardiovascular (CV) disease, no symptoms or physical evidence of cardiovascular disease, no cardiovascular medication, and all echocardiographic measurements within the normal range (Table 5); and 14 patients (referred to as “Reference control with hypertension”) had a history of arterial hypertension, controlled blood pressure (pharmacologically treated to meet JNC 7 criteria i.e., <140/90 mmHg), no left ventricular hypertrophy (Chobanian A V, et al. 2003) and, all echocardiographic measurements within the normal range (Table 5).

TABLE 4
Demographic, Left Ventricular Structure/Function
and MMP/TIMP Data

| | Reference Control | LVH |
|---|---|---|
| Number | 53 | 50 |
| Gender (male/female) | 20/33 | 24/26 |
| Age (years) | 59 ± 1 | 60 ± 2 |
| Systolic blood pressure (mmHg) | 127 ± 2 | 137 ± 3* |
| Diastolic blood pressure (mmHg) | 75 ± 1 | 76 ± 2 |
| End diastolic volume (ml/m²) | 51 ± 2 | 52 ± 2 |
| Ejection fraction (%) | 66 ± 1 | 72 ± 2* |
| LV mass (gm/m²) | 99 ± 3 | 162 ± 6* |
| Volume/mass ratio (ml/g) | 0.54 ± 0.02 | 0.32 ± 0.01* |
| Mitral E/A ratio | 0.95 ± 0.04 | 0.91 ± 0.05 |
| IVRT (msec) | 83 ± 2 | 91 ± 3* |
| E wave Deceleration time (msec) | 208 ± 8 | 234 ± 10* |
| Tissue doppler E′ (cm/sec) | 10.1 ± 0.4 | 7.4 ± 0.4* |
| PCWP (mmHg) | 10 ± 1 | 16 ± 1* |
| MMP-2 (ng/ml) | 1387 ± 39 | 1205 ± 44* |
| MMP-9 (ng/mL) | 13 ± 3 | 26 ± 3* |
| TIMP-1 (ng/mL) | 997 ± 36 | 1291 ± 70* |
| TIMP-2 (ng/mL) | 44 ± 4 | 58 ± 7 |

Abbreviations:
Data are mean + SEM,
LV = Left Ventricular,
LVH = patients with hypertensive left ventricular hypertrophy,
Reference control = subjects with no evidence of cardiovascular disease,
IVRT = isovolumic relaxation time,
PCWP = pulmonary capillary wedge pressure,
MMP = matrix metalloproteinase,
TIMP = tissue inhibitor of MMP,
*= p < 0.05 compared with reference control.

TABLE 5
Reference Controls with and without Hypertension,
LVH with and without CHF

| | Reference | Reference | | |
|---|---|---|---|---|
| | Control | Control | LVH | LVH |
| | without | with | without | with |
| | Hypertension | Hypertension | CHF | CHF |
| Number | 39 | 14 | 23 | 26 |
| Systolic Blood | 126 ± 3 | 131 ± 4 | 138 ± 3* | 133 ± 4 |
| Pressure (mmHg) | | | | |
| Diastolic Blood | 74 ± 2 | 77 ± 2 | 82 ± 2* | 72 ± 2Δ |
| Pressure (mmHg) | | | | |
| End Diastolic | 97 ± 3 | 94 ± 5 | 98 ± 6 | 104 ± 5 |
| Volume (ml) | | | | |
| Ejection Fraction | 65 ± 1 | 66 ± 1 | 70 ± 2* | 73 ± 2* |
| (%) | | | | |
| LV mass (gm/m2) | 94 ± 5 | 101 ± 3 | 160 ± 7*# | 164 ± 7*# |
| Mitral E/A ratio | 0.98 ± .05 | 0.85 ± 0.05 | 0.80 ± .09* | 0.97 ± 0.07Δ |
| Tissue Doppler E′ | 10.0 ± 0.4 | 9.8 ± 0.5 | 8.4 ± 0.4*# | 7.2 ± 0.5*#Δ |
| (cm/sec) | | | | |
| PCWP (mmHg) | 10 ± 1 | 11 ± 1 | 13 ± 2 | 17 ± 2*#Δ |
| PCWP/EDV | 0.09 ± 0.01 | 0.11 ± 0.01 | 0.12 ± 0.01 | 0.17 ± 0.01*#Δ |
| (mmHg/ml) | | | | |
| Ea (mmHg/ml) | 1.50 ± 0.05 | 1.61 ± 0.09 | 1.67 ± 0.10* | 1.45 ± 0.11Δ |
| MMP-2 (ng/ml) | 1383 ± 44 | 1399 ± 84 | 1119 ± 48*# | 1286 ± 73 |
| MMP-9 (ng/ml) | 13 ± 4 | 14 ± 5 | 27 ± 3*# | 24 ± 4*# |
| TIMP-1 (ng/ml) | 1000 ± 42 | 988 ± 76 | 1092 ± 77 | 1364 ± 86*#Δ |
| TIMP-2 (ng/ml) | 42 ± 4 | 48 ± 7 | 58 ± 11 | 59 ± 9 |

Abbreviations:
Data are mean + SEM,
LV = Left Ventricular,
PCWP = pulmonary capillary wedge pressure,
EDV = end diastolic volume,
Ea = effective arterial elastance,
MMP = matrix metalloproteinase,
TIMP = tissue inhibitor of MMP,
LVH = Left Ventricular Hypertrophy,
CHF = Chronic Heart Failure.
Significant differences amongst all 4 groups were analyzed using ANOVA and Tukey's multiple comparison tests,
*= p < 0.05 vs Reference control without Hypertension,
#= p < 0.05 vs Reference control with Hypertension,
Δ= p < 0.05 vs LVH without CHF.

LVH patients were subdivided into two groups based on the presence or absence of CHF. 23 patients with hypertension, controlled blood pressure, with LVH, but no CHF were referred to as “LVH without CHF” (Table 5). The second sub-group consisted of 26 patients with hypertension, controlled blood pressure, LVH, and CHF and was referred to as “LVH with CHF”. All these patients had evidence of CHF defined according to the Framingham criteria (Levy D, et al. 1996), evidence of abnormal relaxation (decreased E′), increased stiffness (increased PCWP and increased PCWP/EDV ratio), a markedly reduced 6 minute walk distance (979±86 feet in LVH with CHF group compared with 1839±60 feet, p<0.05 in the LVH without CHF group), EF≧50%, and therefore, had diastolic heart failure.

Medications used to treat the hypertension were chosen and monitored by the patient's primary physician and not the investigators. These included diuretics, renin-angiotensin-aldosterone antagonists (angiotensin converting enzyme inhibitors, angiotensin II receptor blockers, and aldosterone blockers), direct vasodilators (nitrates, hydralazine), alpha adrenergic blockers, central nervous system blockers, aspirin, beta adrenergic receptor blockers, and calcium channel blockers. The mean duration of antihypertensive treatment was 6.4±1.5 years.

Echocardiographic Methods: Echocardiograms were performed using a Sonos 5500 system with an S-4 MHz transducer. Measurements were made using American Society of Echocardiography criteria (Sahn D J, et al. 1978; Schiller N B, et al. 1989). LV and left atrial volumes were calculated using the method of discs (Schiller N B, et al. 19). LV mass was calculated using the formula of Reichek and Devereux (Devereux R B, et al. 1986). Doppler measurements of mitral inflow E and A wave velocity, the E/A ratio, E wave deceleration time, and isovolumic relaxation time (IVRT) were made. Tissue Doppler (lateral mitral annulus) measurement of mitral E′ and A′ wave velocity were made. Pulmonary capillary wedge pressure (PCWP) was calculated using the formula: 2+⅓ E/E′ (Nagueh S F, et al. 1998). Effective arterial elastance (Ea) was calculated using the formula: end systolic pressure/stroke volume.

MMP/TIMP Plasma Measurements: Gelatinases (MMP-2 and MMP-9), collagenase (MMP-13); and tissue inhibitors of MMPs (TIMP-1 and TIMP-2) were examined using 2-site enzyme-linked immunosorbent assay (ELISA) kits (Amersham Pharmacia Biotech, Buckimghamshire, UK). Plasma and the respective MMP standards were added to precoated wells containing the antibody to the MMP or TIMP of interest and washed. The resultant reaction was read at a wavelength of 450 nm (Labsystems Multiskan MCC/340, Helsinki, Finland). Because MMP-13 was found in very low levels in the plasma, the MMP-13 results were divided into detectable and non-detectable.

Statistical Analysis: MMP and TIMPs were measured every 2 hours for a 6 hour period in order to calculate a coefficient of variance for MMP/TIMP measurements between and within individual subjects in a subgroup of reference control subjects (n=20) using a one-way random effects ANOVA. Then the coefficient was calculated as the square root of the within person mean square error times 100. The intra-patient coefficient of variation for MMP-2=11.2±1.1%, TIMP-1=8.5±2.2% and TIMP-2=14.3±1.7%. An intra-assay coefficient of variation quantifying variation in the assay technique itself was less then 6% for all the MMP and TIMPs.

Initially, comparisons between reference controls versus LVH subjects were made using a 2-tailed Student t test. Subsequently, comparisons between all 4 groups (reference control with versus without hypertension versus LVH with versus without CHF) were analyzed using ANOVA and Tukey's multiple comparison tests. A p value of <0.05 was considered significant. Simple linear regression was used to examine the relationship between MMP and TIMP levels and measurements of LV structure and function. Mantel Hanzel chi square and receiver operating curves were used to evaluate the association between MMP-13 and TIMP-1 levels and presence of LVH and CHF. The potential effects of the medications on structure, function, or plasma data were examined first by a univariate then by a multivariate regression analysis. The structure, function, MMP, or TIMP measurement was the dependent variable with the medication entered as a dummy variable. A single drug was examined, and then drugs in combination were examined.

The research protocol used in this study was reviewed and approved by the institutional review board at the Medical University of South Carolina. Written informed consent was obtained from all participants. The authors had full access to the data and take responsibility for its integrity. All authors have read and agree to the manuscript as written.

2. Results

Reference Control Versus LVH

Structure/Function Data: The reference control subjects had a similar age and gender distribution as the LVH subjects (Tables 3 and 4). Compared to reference control, LVH had higher systolic blood pressure, significant concentric remodeling as evidenced by a 60% greater LV mass index, no difference in end diastolic volume, and a 40% lower LV end diastolic volume versus mass ratio. Compared to reference control, LVH had significant abnormalities in indices of LV diastolic relaxation and LV diastolic stiffness: increased IVRT, increased E wave deceleration time, decreased E′, increased pulmonary capillary wedge pressure, and increased PCWP versus LV end diastolic volume ratio (0.16±0.01 mmHg/mL in LVH) compared to reference control (0.09±0.01 mmHg/mL, p<0.05), indicating that there was an increase in the LV instantaneous end diastolic operating stiffness.

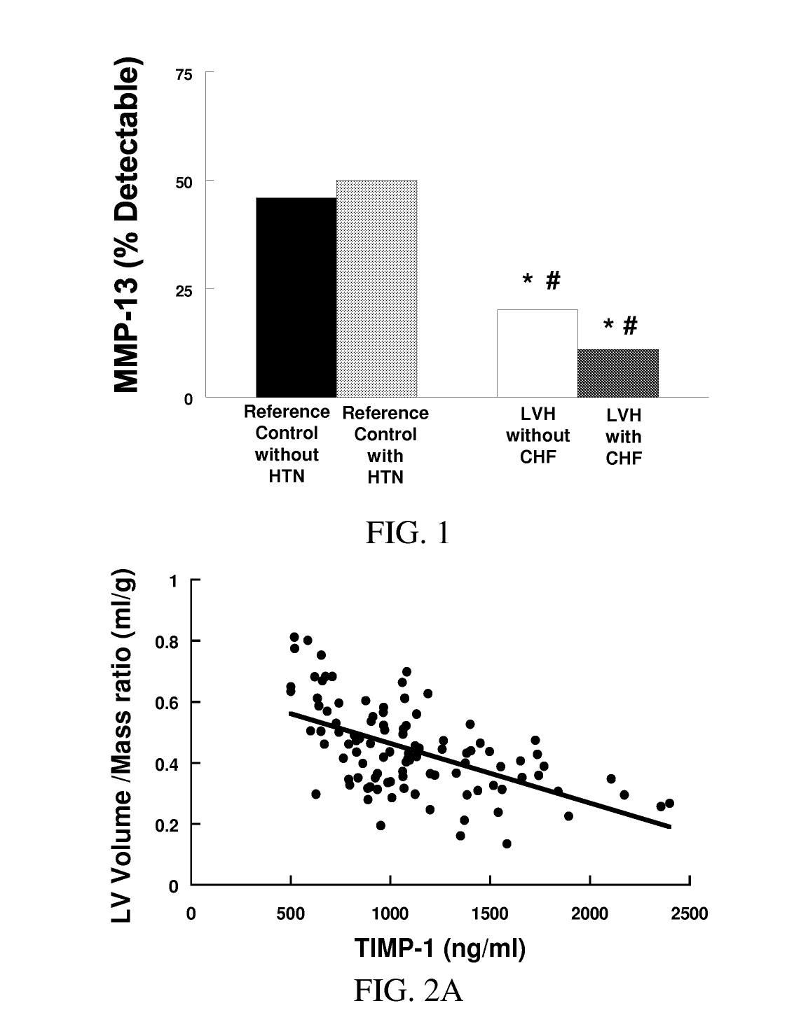

MMP and TIMP plasma profiles: Compared to reference control, MMP-2 was decreased and MMP-9 was increased in LVH. Significant differences were found in MMP-13 detectability (FIG. 1). Forty-seven percent of the reference control subjects had a detectable level of MMP-13, while MMP-13 was detectable in only 15% of the LVH subjects (χ²=17.89, p<0.001, odds ratio=0.24). Plasma TIMP-1 was significantly increased in LVH compared to reference control. TIMP-2 and the MMP-9/TIMP-1 and MMP-2/TIMP-2 ratios were not different between reference control and LVH.

Reference Control without Hypertension Versus Reference Control with Hypertension

Structure/Function Data: Reference control subjects without hypertension served as the age and gender matched reference control group for comparison to the reference control with hypertension, the LVH without CHF, and the LVH with CHF groups. There were no significant differences in any demographic parameter or any echocardiographic measurement of LV structure or function between reference controls without hypertension versus reference control with hypertension (Tables 3 and 4). Left atrial maximum volume (LAMV) and emptying fraction (LAEF) were similar in reference control without hypertension (LAMV=40±2 ml, LAEF=42±3%) compared to reference control with hypertension (LAMV=42±4 ml, LAEF=43±2%).

MMP and TIMP plasma profiles: There were no significant differences in any MMP or TIMP plasma level between reference control subjects without hypertension versus reference control with hypertension.

LVH without CHF Versus LVH with CHF

Structure/Function Data: There were no significant differences in systolic blood pressure, LV volume, or mass between LVH without CHF and LVH with CHF subjects (Table 5). However, diastolic function was significantly more impaired in LVH with CHF compared to LVH without CHF. Indices of diastolic relaxation were slower, diastolic stiffness was greater and filling pressures were higher in LVH with CHF compared to LVH without CHF. In particular, in the LVH without CHF patients, tissue Doppler E′ was decreased (8.4±0.4 cm/sec with 95% confidence intervals (CI) of 7.4, 9.3) compared with reference control without hypertension (10±0.4 cm/sec, 95% CI=9.3,11) and reference control with hypertension (9.8±0.5 cm/sec, 95% CI=8.1,11). E′ fell further in LVH with CHF (7.2±0.5 cm/sec, 95% CI=6.2, 8.3). In the LVH without CHF patients, PCWP was unchanged (13±2 mmHg, 95% CI=10.5, 15.2) compared with reference control without hypertension (10±1 mmHg, 95% CI=9.3, 10.6) and reference control with hypertension (11±1 mmHg, 95% CI=9.1, 12.2) but increased in LVH with CHF (17±2 mmHg, 95% CI=15.2, 17.7). The PCWP versus LV end diastolic volume ratio was not changed in the LVH without CHF patients but was significantly increased in the LVH with CHF patients. Effective arterial elastance was increased in LVH without CHF and was decreased in LVH with CHF. LAMV was increased in LVH without CHF (LAMV=53±4 ml, p<0.05 compared with reference control) and increased further in LVH with CHF (LAMV=70±5 ml, p<0.05 compared with LVH without CHF). LAEF was unchanged in the LVH with CHF (LAEF=42±3%, p<0.05 compared with reference control) but increased in LVH with CHF (LAEF=48±2% compared with LVH without CHF).

MMP and TIMP plasma profiles: There were no significant differences in MMP-2, -9, -13, TIMP-2 or MMP/TIMP ratios in LVH without CHF compared to LVH with CHF (FIG. 1). However, TIMP-1 was significantly increased in LVH with CHF (1364±86 ng/ml, 95% CI=1185, 1543) compared to LVH without CHF (1092±77 ng/ml, 95% CI=933,1252). In fact, TIMP-1 was elevated only in subjects with CHF. TIMP-1 was unchanged in the LVH without CHF patients compared with reference control without hypertension (1000±42 ng/ml, 95% CI=915,1085) and reference control with hypertension (988±76 ng/ml, 95% CI=824,1152).

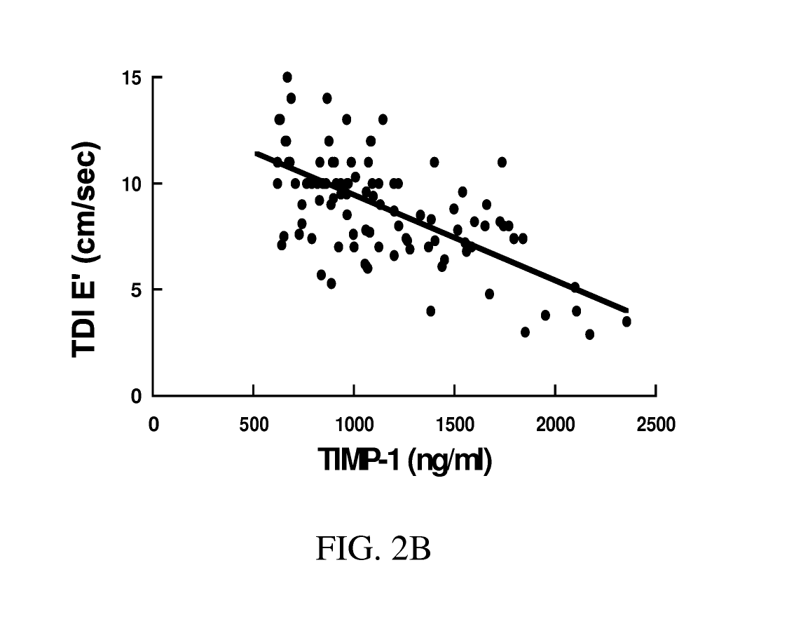

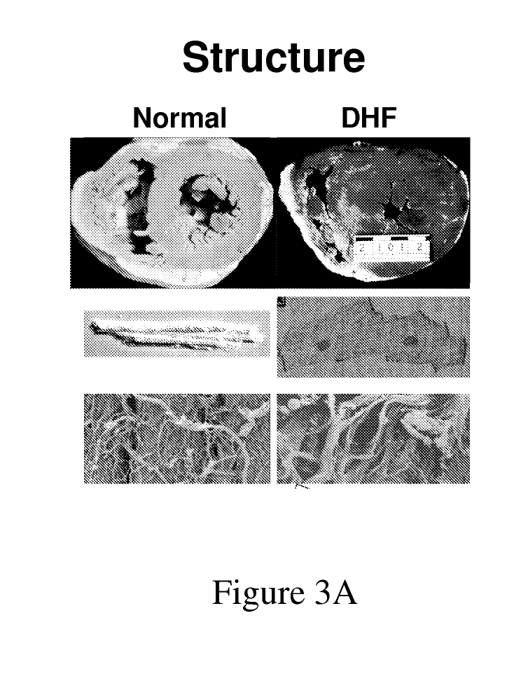

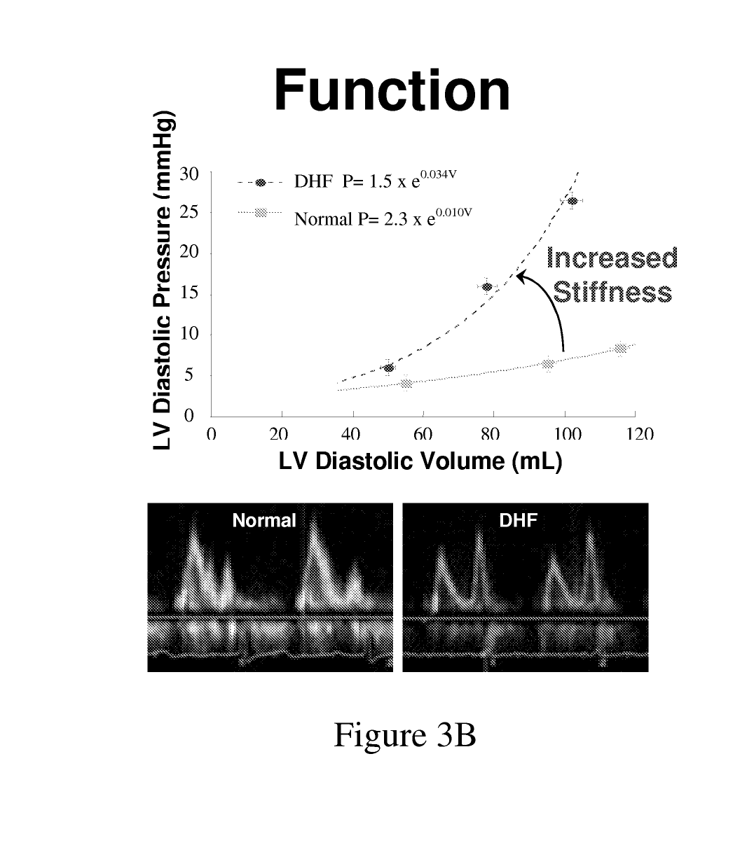

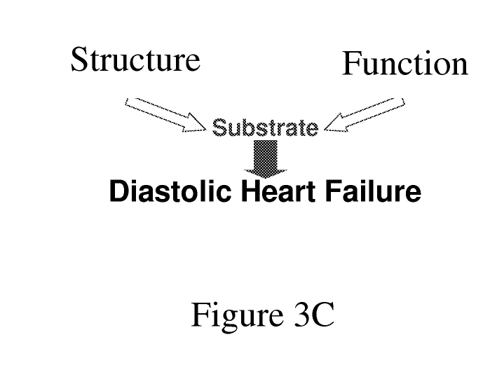

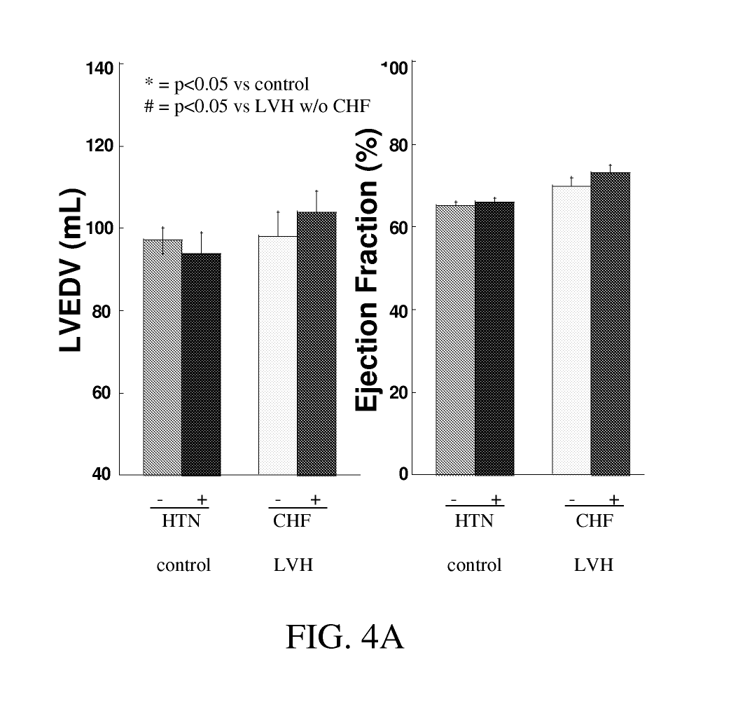

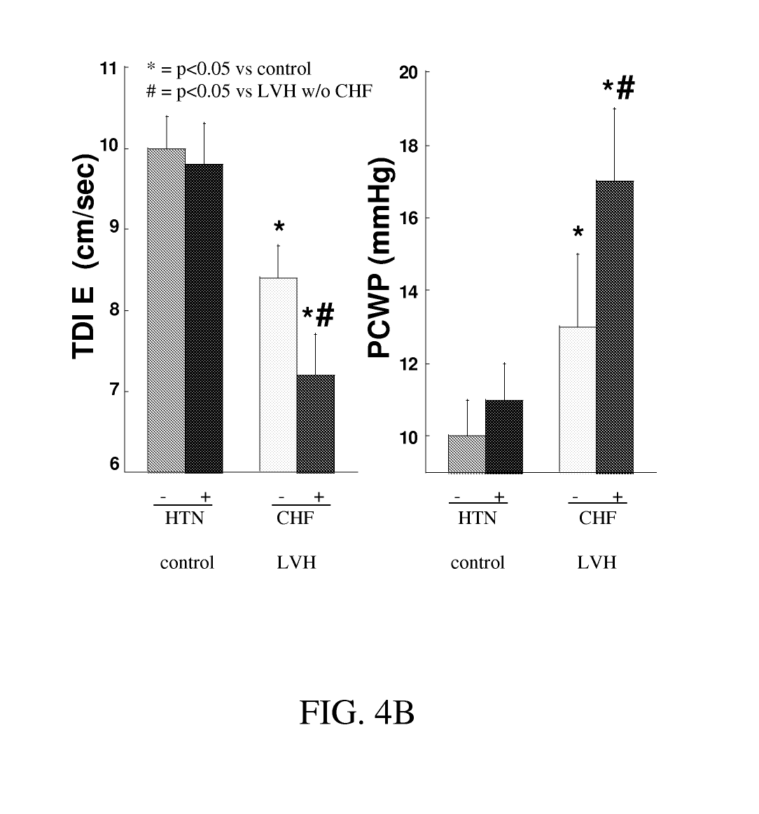

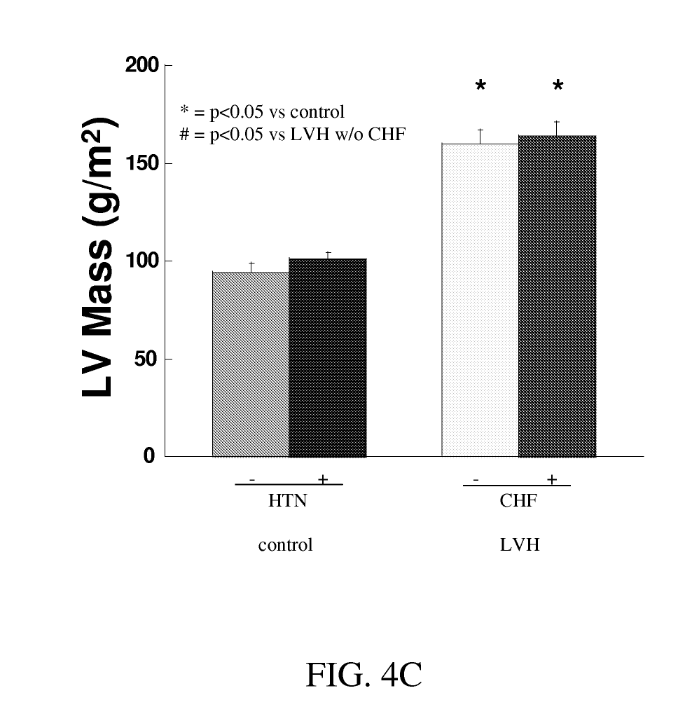

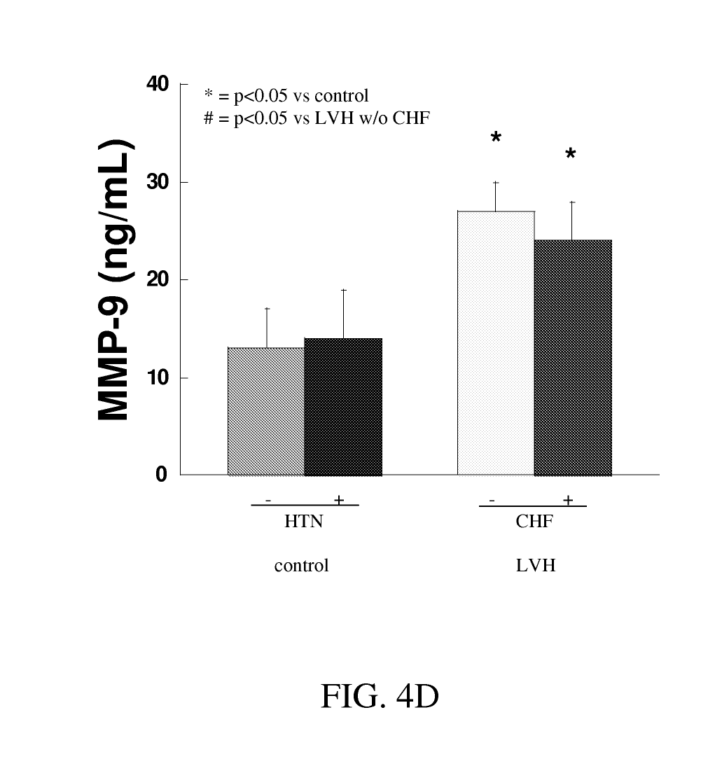

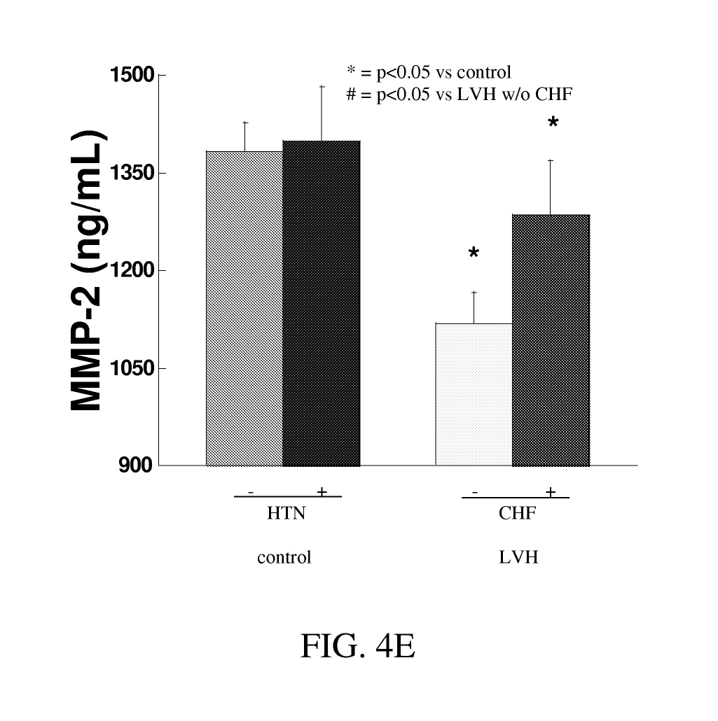

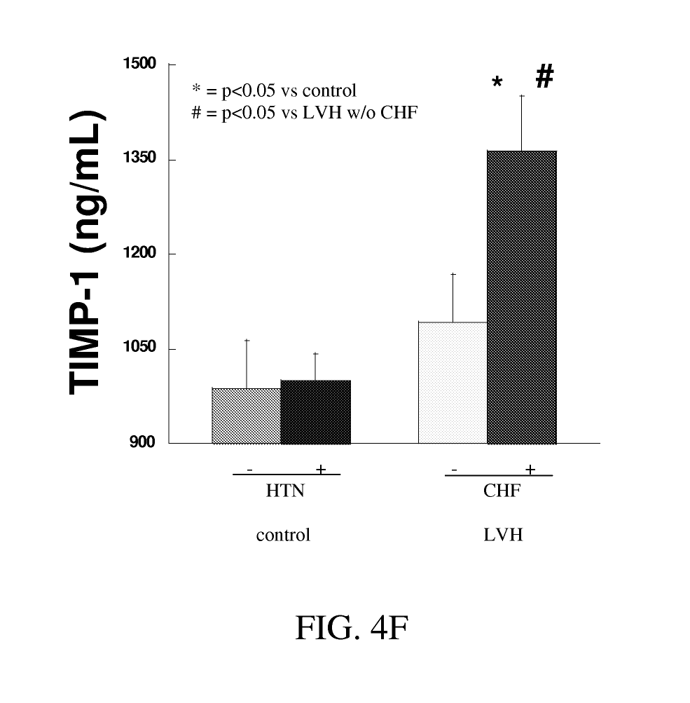

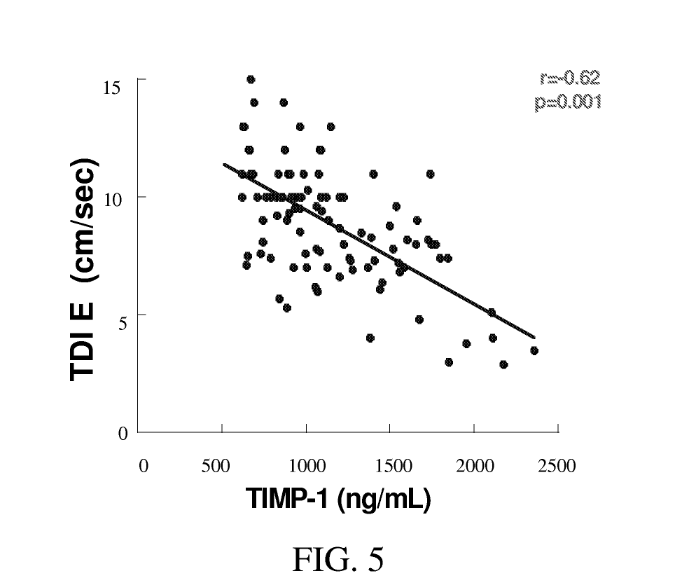

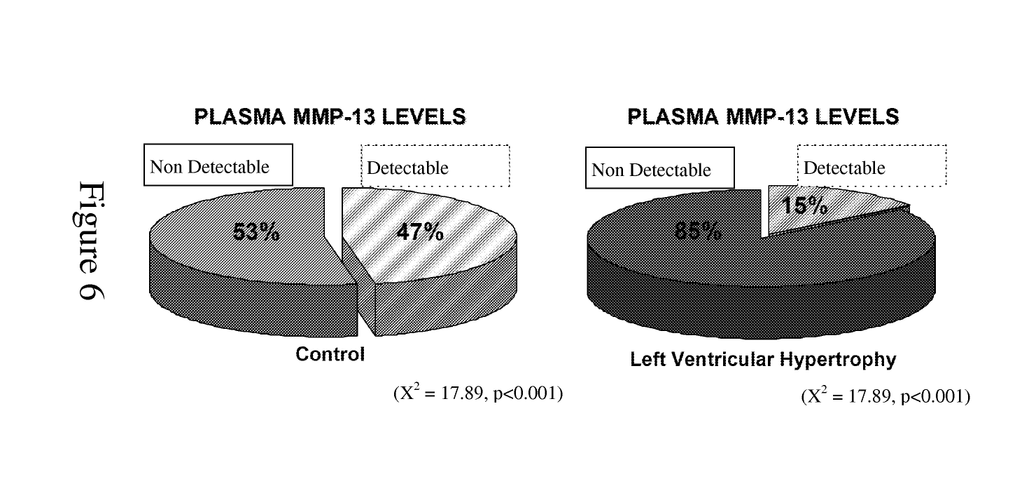

Relationship between MMP and TIMP plasma profiles and LV structure and function: There was a significant relationship between TIMP-1 and the extent of LV remodeling. As TIMP-1 increased, LV mass increased (r=0.30, p=0.005) and the volume/mass ratio fell (r=−0.56, p=0.001, FIG. 2A). There was a significant relationship between TIMP-1 and the extent of diastolic dysfunction. As TIMP-1 increased, the mitral E/A ratio decreased (r=−0.22, p<0.027), E′ fell (r=−0.62, p=0.001, FIG. 2B), and the PCWP increased (r=0.28, p=0.013). Finally, there is a significant relationship between extent of CHF and TIMP-1 levels. The mean value of TIMP-1 was higher in LVH subjects with CHF who were NYHA class III versus class II. Having a TIMP-1 level of >1200 ng/ml was predictive of having LVH with CHF (χ²=4.6, p=0.03, specificity=88% and positive predictive value=94%, odds ratio=3.54, 95% confidence intervals=1.08, 11.50). The area under the receiver operator curve (ROC) was 0.71.

There was no relationship between the use of a specific medication and differences in LV structure, function or plasma MMP/TIMP profiles between groups. Specifically, there were no differences in any MMP or TIMP level between patients grouped by any medication or combination of medications. None-the-less, it is recognized that this study was not powered sufficiently to completely address the effects of drugs on LV structure, function or plasma MMP/TIMP profiles. Therefore, these data and analysis must be interpreted with appropriate caution.

3. Discussion

There were 3 unique findings in this study: 1) patients with hypertension but normal LV structure and function had a normal MMP/TIMP profile, 2) changes in MMP and TIMP profiles which favor decreased ECM degradation (decreased MMP-2, -13, increased TIMP-1) were associated with LV hypertrophy and diastolic dysfunction, and 3) increased TIMP-1 predicted the presence of CHF.

While pleotropic in their substrates and actions, changes in myocardial MMPs and TIMPs have predictable effects on the ECM (Spinale, F G. 2002; Chapman R E, et al. 2004). For example, MMP-2 (a gelatinase) degrades basement membrane proteins, fibrillar collagen peptides, and newly synthesized collagen fibers. In the current study, MMP-2 was significantly decreased in patients with hypertensive LVH. MMP-9 (a gelatinase) has the same structural protein substrates as MMP-2 but has a much lower level of activity. However, MMP-9 has significant affects on important biologically active proteins/peptides such as TGF-β, and other “pro-fibrotic” proteins and pathways. Activation of pro-fibrotic pathways by increased MMP-9 would be expected to increase ECM accumulation. Thus, the decreased MMP2 and increased MMP-9 levels found in the LVH patients in the current study may be one factor contributing to the observed structural and functional changes seen in hypertensive heart disease.

MMP-13 is a collagenase that is found in very low levels in the plasma and is difficult to quantify accurately even with a high sensitivity assay. Therefore, in the current study, rather then reporting MMP-13 as a quantitative value, the results were dichotomized Detectable MMP-13 in the plasma of patients with LVH was greatly reduced and was further reduced in patients with LVH and CHF. The reduction in this collagenolytic enzyme would be expected to cause reduced fibrillar collagen turnover, reduced degradation, and increased ECM accumulation.

MMP activity is regulated at several levels that not only includes transcriptional regulation, but also includes post-translational modification such as TIMP binding. The TIMPs bind to active MMPs in a 1:1 relationship, inhibit MMP enzymatic activity and thereby form an important control point with respect to net ECM proteolytic activity (Spinale, F G. 2002; Chapman R E, et al. 2004; Brew K, et al. 2000). The current study showed that plasma levels of TIMP-1 increased in patients with LVH and CHF. As a result, the balance between MMPs and TIMPs was altered in favor of reduced ECM proteolytic activity which would therefore facilitate ECM accumulation. There are four known TIMPs, and the transcriptional regulation of these molecules is not homogeneous (Brew K, et al. 2000). Discordant levels of TIMPs have been observed in both animal models of heart failure and in patients with cardiomyopathic disease (Wilson E M, et al. 2002; Stroud R E. 2005). In the current study, a robust increase in TIMP-1 was observed in LVH patients with CHF. In contrast, only a small increase in TIMP-2 was observed in LVH patients either with or without CHF. These observations likely underscore the different functions and regulatory pathways for TIMPs in the LV remodeling process. A unique finding of the present study was that a specific type of TIMP, TIMP-1 was strongly associated with the development of CHF. In patients with LVH and CHF, it is not clear whether the increased TIMP-1 levels contributed to the development of CHF or was the result of its development. What is clear however, is that increased TIMP-1 was uniquely present in patients with LVH and CHF and plasma TIMP-1 values>1200 ng/ml was predictive of the presence of CHF. Therefore, this plasma analyte should be considered in the development of diagnostic criteria for heart failure with a normal ejection fraction (diastolic heart failure) and for design of novel therapeutic management strategies for diastolic heart failure. However, it is recognized that the partition value of TIMP-1=1200 ng/ml was chosen in a “post-hoc” rather then a prospective fashion. Therefore, the validity of its predictive value must be interpreted with appropriate caution and confirmed in additional studies which use a large, prospective serial study design.

The changes in MMP/TIMPs that occur in patients with hypertensive heart disease can effect growth regulation in both the extracellular and the cardiomyocyte compartments which together result in concentric LV hypertrophy and increased collagen content. Collagen homeostasis is determined by the balance between synthesis, post-translational modification and degradation. In hypertensive heart disease, Diez et al and others have shown that increased collagen content was associated with increased plasma markers of collagen synthesis, decreased collagen degradation and decreased collagen turnover (Diez J, et al. 2002; Lopez B, et al. 2001a; Lopez B, et al. 2001b). Changes in the MMP/TIMP profiles found in the current study disclose potential mechanisms by which changes in synthesis, degradation and turnover may take place.

While there are many determinants of LV structural remodeling, blood pressure is one of the most important. However, data from the current study indicate that even after blood pressure has been adequately controlled, ongoing changes in MMPs and TIMPs predict, probably determine, and are certainly associated with persistent concentric remodeling, LVH and diastolic heart failure. Regression of LVH requires appropriate remodeling of the ECM including degradation and turnover of ECM components (particularly the basement membrane proteins) and alterations the cardiomyocyte-matrix interactions. The current study showed that patients with hypertensive LVH had persistent abnormalities in specific MMP (decreased MMP-2) and TIMP (increased TIMP-1) profiles which would be expected to favor continued cardiomyocyte-basement membrane-matrix connections and not the ECM turnover necessary to accommodate LV mass regression. It seems likely therefore, that the ongoing changes in MMPs and TIMPs seen in the current study contribute to the phenotypic and structural changes present in hypertensive heart disease.

The current study utilized plasma levels of MMPs and TIMPs as surrogate markers to reflect changes in myocardial levels of these enzymes and peptides. MMP activation and TIMP binding is a compartmentalized process that occurs within the myocardial interstitium (Spinale, F G. 2002; Chapman R E, et al. 2004). Thus, plasma levels do not necessarily reflect the net ECM proteolytic activity that occurs within the myocardium. Differences in plasma MMP and TIMP levels observed between reference control and patients with hypertensive heart disease in the current study are likely to reflect differences at the myocardial level (Joffs C, et al. 2001; Yarbrough W M, et al. 2003; Lindsey M L, et al. 2003). It is possible that the myocardium is not the only source of MMPs and TIMPs in LVH patients. Therefore, measurements of plasma MMP and TIMP levels represent the summation of MMPs and TIMPs released from both cardiac as well as non-cardiac sources. However, the specific exclusion criteria utilized in the current study helped to eliminate significant changes in the major non-cardiac sources of MMPs and TIMPs. Never-the-less, it must be recognized that patients with hypertension and LVH, with or without chronic heart failure, can have changes in other non-cardiac tissues, such as the kidneys and the vasculature, that can contribute to MMP and TIMP release into the plasma. The findings of the current study demonstrate differences in plasma MMP and TIMP levels between reference control and LVH patients.

Conclusion: A specific pattern of changes in the ECM proteolytic system was associated with each structural, functional, and/or clinical manifestation of hypertensive heart disease. Subjects with adequately controlled blood pressure with no structural or functional changes in the left ventricle did not have any changes in the MMP/TIMP signature. However, patients with LVH in spite of adequate blood pressure control had decreased MMP-2 and -13. Increases in TIMP-1 were found in patients with LVH and CHF. In particular, the transition between hypertensive LVH and the development of CHF is heralded by changes in MMPs and TIMPs such as an increase in TIMP-1>1200 ng/ml or the absence of MMP-13. However, the current study had a limited sample size, used a cross-sectional design, and did not perform serial studies over time. None-the-less, the data from the current study indicate that the observed stochastic changes in MMP/TIMPs play an important role in the manifestations of hypertensive heart disease. Understanding this ECM dependent pathophysiology provides improved diagnosis and treatment of patients with hypertensive heart disease.

Clinical Perspective: Chronic arterial hypertension is a common cause of LV concentric hypertrophy, decreased relaxation rate and increased stiffness. The structural and functional changes caused by hypertension result from changes to both of the principle constituents of the myocardium, the cardiomyocyte and particularly the extracellular matrix (ECM). These LV structural and functional changes create the substrate necessary for the development of diastolic heart failure (DHF). However, what controls these changes in the ECM, whether blood pressure control alone can prevent or reverse these changes, and whether knowledge of the ECM-control mechanisms would aid diagnosis or treatment of hypertensive heart disease is unknown. The current study showed that changes in the pattern of specific ECM proteolytic proteins/peptides (MMPs and TIMPs) were associated with each structural, functional, and clinical manifestation of hypertensive heart disease. Subjects with adequately controlled blood pressure with no LV structural or functional changes did not have any changes in the MMP/TIMP signature. Therefore, treatment of hypertension can prevent changes in the ECM and the ECM proteolytic system. However, patients with residual or resistant LVH, in spite of adequate blood pressure control, had abnormal MMPs. The development of DHF was heralded by an increase in TIMP-1>1200 ng/ml. These data indicate that regression of LVH and prevention of DHF are dependent on more than just changes in blood pressure alone, and may need to target and normalize changes in MMP/TIMPs. Understanding this ECM dependent pathophysiology provides improved diagnosis and treatment of patients with hypertensive heart disease.

### B. Example 2

### Matrix Metalloproteinases/Tissue Inhibitors of Metalloproteinases: Relationship between Changes in Proteolytic Determinants of Matrix Composition and Structural, Functional, and Clinical Manifestations of Hypertensive Heart Disease

1. Methods

Study Enrollment: Table 6 shows the study enrollment. The exclusion criteria were a history of myocardial infarction, cardiomyopathy, valvular or wall motion abnormalities, arrhythmia, infiltrative cardiac disease, EF<50%, uncontrolled hypertension (SBP>140 or DBP>90), or systemic disease that affect MMP/TIMP plasma profiles. The inclusion criteria for controls and controls with HTN were men and women age 18-90 years without evidence of structural cardiovascular disease. The inclusion criteria for LVH and LVH with CHF were men and women age 18-90 years with established LV hypertrophy by echiocardiography (wall thickness of >1.2 cm or LV mass Index>125 g/m²).

TABLE 6
Study Enrollment

| | Control | | LVH | |
|---|---|---|---|---|
| | −HTN | +HTN | −CHF | +CHF |
| Number | 39 | 14 | 23 | 26 |
| Age | 59 ± 2 | 60 ± 2 | 56 ± 2 | 64 ± 2 |
| SBP (mmHg) | 126 ± 3 | 131 ± 4 | 138 ± 3 | 133 ± 4 |
| DBP (mmHg) | 74 ± 2 | 77 ± 2 | 82 ± 2 | 72 ± 2 |

Echocardiography measurements: standard to dimensional echocardiography was used.

Echocardiography calculations: LV volume was calculated by the method of discs. LV Mass was calculated by the Penn Method. PCWP was calculated as 2+1.3×(E/Ea).

MMP/TIMP Plasma Measurements: Plasma measurements were obtained by enzyme-linked immunosorbent assay (ELISA) (Ammersham Pharmacia Biotech) for the gelatinases MMP-2 and MMP-9, the collagenase MMP-13, and the TIMPS TIMP-1 and TIMP-2.

2. Results

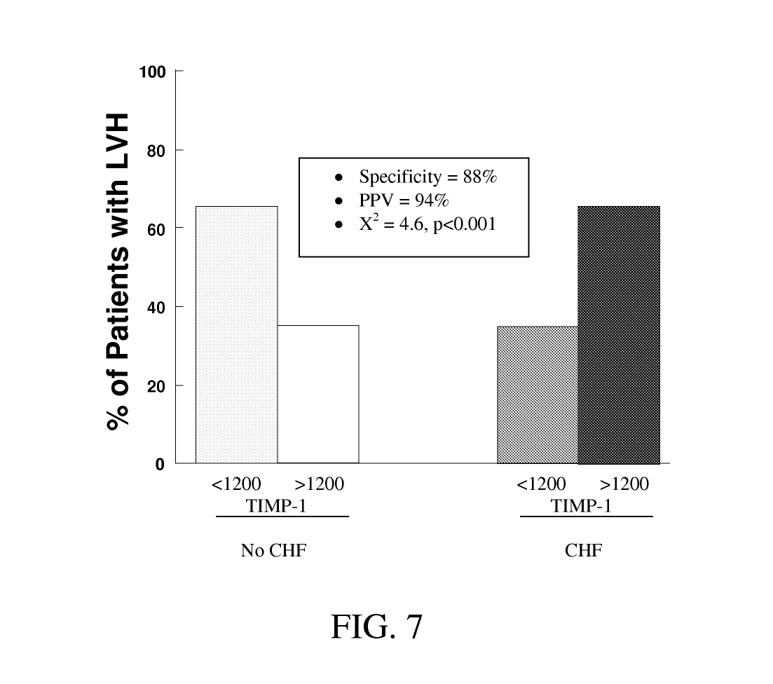

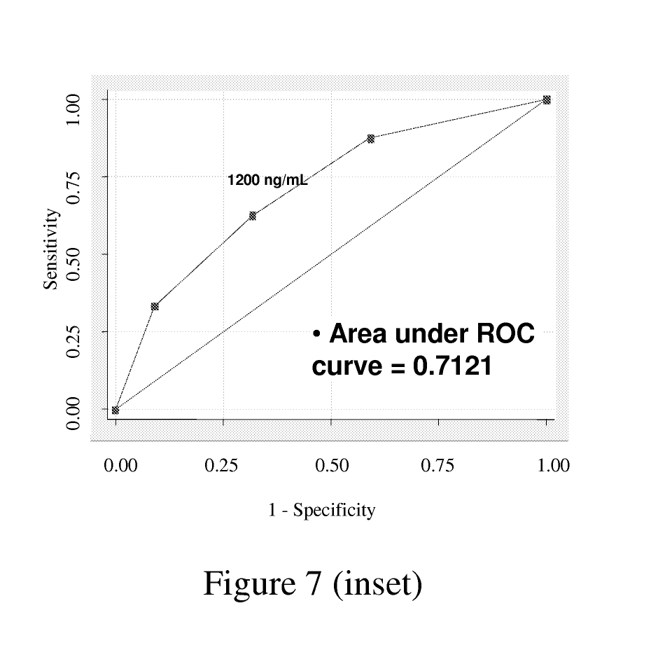

FIGS. 7-11 show the results of the study.

3. Conclusions

Patients with HTN but normal LV structure and function had a normal MMP/TIMP profile. Changes in MMP/TIMP profiles which favor decreased ECM degradation were associated with LV hypertrophy and diastolic dysfunction. Increased TIMP-1 predicted the presence of CHF. Changes in the myocardial extracellular matrix proteolytic system are measurable using plasma assays of selected MMPs and TIMPs. Each manifestation of hypertensive heart disease is associated with a specific pattern of changes in the ECM proteolytic system. Hypertensive patients with structural remodeling, diastolic dysfunction and/or clinical CHF are characterized by a decrease in the MMPs and an increase in TIMPs.

### C. Example 4

### Criteria for Differentiating, Predicting and Diagnosing Heart Failure in Patients with Hypertension

Provided in Table 7, a clear set of normal values for human subjects within the age range and across genders is provided. There has been no previously compiled list of normal reference values for MMPs/TIMPs that are as inclusive as this and furthermore provides for normal reference ranges since age matched subjects, free from cardiovascular disease were included. Moreover, novel stoichiometric ratios for MMP/TIMP profiles are provided which will prove to hold important diagnostic and prognostic information as detailed in subsequent tables. These data were collected and analyzed from over 100 subjects.

TABLE 7
Normal Human* Reference Ranges
MMP/TIMP Plasma Levels (ng/mL)

| | |
|---|---|
| MMP-2 | 1000-1500 |
| MMP-9 | 0-20 |
| MMP-7 | 0-5 |
| MMP-13 | 0-10 |
| MMP-8 | 0-3 |
| TIMP-1 | 800-1000 |
| TIMP-2 | 25-50 |
| TIMP-4 | 0-2 |

MMP-9/TIMP Ratios

| | |
|---|---|
| MMP-9/TIMP-1 | 7-15 |
| MMP-9/TIMP-2 | 100-500 |
| MMP-9/TIMP-4 | 1-10 |

*Normal Adults Age 25-70 years

Table 8 presents the MMP and TIMP values in absolute terms, the MMP/TIMP ratios in absolute terms, and the percent changes from normal reference values based upon the absolute terms, in patients with well managed blood pressure, but carry a diagnosis of hypertension. These values were collected as described within the body of the original application. A unique plasma profile, which would not be predicted from past reports in animal studies or the limited clinical studies published previously is demonstrated. This unique profile includes a fall in MMP-2, no change in MMP-9, non-detectable (below sensitivity of any assay system currently used) for MMP-13, and robustly increased levels of TIMP-1. Moreover, an increase in the cardiovascular specific marker for TIMP-4 could be demonstrated. These changes in MMP and TIMP profiles are unique to patients with hypertension and demonstrate early changes occurring within the heart tissue of these patients. This unique and specific profile can be used to guide therapy in order to minimize these changes in MMP and TIMP profiles from normal subjects. Moreover, these plasma profiles can be used for generalized screening for at risk patient populations and identify patients that are at risk for future adverse events.

TABLE 8
Diagnostic for Hypertensive Heart Disease
Plasma MMP/TIMP Levels (ng/mL)*

| | |
|---|---|
| MMP-2 | <1000 |
| MMP-9 | 25-50 |
| MMP-7 | 0-5 |
| MMP-13 | 0-5 |
| MMP-8 | 0-3 |
| TIMP-1 | >1000 |
| TIMP-2 | >50 |
| TIMP-4 | >2 |

Plasma MMP/TIMP Ratios

| | |
|---|---|
| MMP-9/TIMP-1 | <5 |
| MMP-9/TIMP-2 | <100 |
| MMP-9/TIMP-4 | <3 |

*Patient diagnosed with high blood pressure and under proper medical management

Table 9 demonstrates plasma profiles for MMPs and TIMPs that emerge in patients with heart failure secondary to hypertensive heart disease. These data were compiled from studies provided in the initial application. This past study demonstrated that the differentiation of the presence and absence of heart failure in hypertensive patients could be obtained by the loss of a signal for MMP-13 and the robust increase in TIMP-1. In fact, receiver operator curves (ROC) for prediction and diagnosis for heart failure were provided previously. In marked contrast to patients with heart failure secondary to a myocardial infarction (heart attack), MMP-9 levels are normal or below normal. The differentiation between these two disease states is possible and provided in an upcoming table. Moreover, utilizing a cardiovascular specific marker, TIMP-4, it could be demonstrated that this was increased in patients with hypertensive heart disease and that this provided cardiovascular specificity to the plasma profile-never demonstrated previously. These data provide the first differential profile for identifying through plasma markers, patients suffering from heart failure due to hypertensive heart disease. This is an important issue as t treatment modalities differ based upon the underlying cause of the heart failure. How these new data could be used to guide therapy and clinical decision making was provided in the initial application.

TABLE 9
Hypertensive Patients at Increased Risk for Heart Failure*
Plasma MMP/TIMP Levels (ng/mL)

| | |
|---|---|
| MMP-2 | <500 |
| MMP-9 | 0-20 |
| MMP-7 | 0-5 |
| MMP-13 | ND (not detectable) |
| MMP-8 | 0-3 |
| TIMP-1 | >1500 |
| TIMP-2 | >100 |
| TIMP-4 | >6 |

Plasma MMP/TIMP Ratios

| | |
|---|---|
| MMP-9/TIMP-1 | <2 |
| MMP-9/TIMP-2 | <50 |
| MMP-9/TIMP-4 | <0.25 |

*Patient diagnosed with high blood pressure and under proper medical management

The unique plasma signature that was developed in this application and presented in the supporting material provides for the first time an ability to differentiate the underlying causes for a patient presenting for heart failure. Specifically, as shown in Table 10, a unique and very different plasma profile emerges from a patient at risk for developing, or presenting with heart failure secondary to a myocardial infarction or that in patients with heart failure secondary to hypertension. These data were compiled from completed studies. Thus, differential diagnoses can be made on these profiles and more importantly more specific clinical decision making and therapeutic strategies considered. Examples of clinical applications for this profile and how these would be utilized in clinical decision making was provided in the initial application.

TABLE 10
Differential Diagnosis of Systolic (Post-MI) or
Diastolic (Hypertensive Heart Disease) Heart Failure*

| | SystolicHF | Diastolic HF |
|---|---|---|
| Plasma MMP/TIMP Profiles | | |
| MMP-2 | ↓ | ↓ |
| MMP-9 | ⇑ | → |
| MMP-7 | → | → |
| MMP-13 | → | ↓ or ND |
| MMP-8 | ⇑ | → |
| TIMP-1 | ⇑ | ⇑⇑ |
| TIMP-2 | ⇑ | ⇑⇑ |
| TIMP-4 | ↓ | ⇑⇑ |
| Plasma MMP/TIMP Ratios | | |
| MMP-9/TIMP-1 | ⇑ | ↓ |
| MMP-9/TIMP-2 | ⇑ | ↓ |
| MMP-9/TIMP-4 | ⇑ | ↓ |

### D. Example 5

### Analysis of Additional Biomarkers as Indicators of Left Ventricular Remodeling and Congestive Heart Failure

Arterial hypertension (HTN) can cause ventricular (LV) remodeling, alterations in cardiac function, and the development of chronic heart failure (CHF). Changes in the composition of the myocardial extracellular matrix (ECM) are causally related to these structural, functional, and clinical outcomes. Regulation of ECM structure and function is determined by the expression and activation of a large family of matrix metalloproteinases (MMPs) and tissue inhibitors of MMPs (TIMPs), and ECM signaling molecules such as cardiotrophin and osteopontin. In addition, collagen type I and III propeptides (PIIINP, PINP) can be reflective of ECM synthesis and turnover. However, a systematic, prospective evaluation in which all of these ECM biomarkers are quantified and related to LV remodeling and CHF in patients with HTN has never been undertaken. This disclosure identifies new findings that measuring multiple biomarkers, at one time in one blood sample, provides unique predictive, diagnostic and prognostic information.

The scope of this new discovery is that additional biomarkers: Cardiotrophin, Osteopontin, soluble receptor for advanced glycated end products (sRAGE), collagen telopeptides (PIIINP, PINP) and propetide (CITP) have been integrated into a study of hypertensive patients with and without heart failure. We have identified that using these biomarkers in a combinatorial fashion with those already uniquely identified previously (MMPs/TIMPs) provides a unique insight into the progression and detection of hypertensive heart disease.

This example focuses upon identifying novel biomarkers, which can be obtained from peripheral blood samples, which provide predictive and prognostic information regarding hypertensive heart disease. The objectives were to measure a finalized list of candidate biomarkers, which are listed in Table 31, on a set of archived plasma samples collected from reference normal and hypertensive subjects. The archived samples were from a total of 150 patients. This example provides a description of these collected samples with respect to demographics, left ventricular (LV) function, and biomarker profiles.

1. Archived Subjects

The subjects were stratified into 3 classifications: non-hypertensive subjects (also free from cardiovascular disease, or other confounding disease; n=62); patients with hypertension (HTN) but absent of left ventricular hypertrophy (no LVH; based upon echocardiography; n=21); and patients with established LVH (n=67). The patients with LVH were further dichotomized with respect to having clinical symptoms of congestive heart failure (CHF), using established clinical criteria. Plasma for all of these subjects were subjected to multiplex suspension array methodology, high performance enzyme linked immunoassay (ELISA), or radio-immunoassay dependent upon the analyte of interest (Table 31). Strict criteria for standard curves, outliers, and calibration performance were followed for all assays.

TABLE 31
Biomarker Assay Characteristics

| | | | | Dynamic |
|---|---|---|---|---|
| | | | Fit Probability/ | Range |
| Biomarker | Assay | Equation Type | r value | (ng/mL) |
| MMP 2 | Multiplex | Logistic-5 PL | 0.6174 | 0.0794-59.69 |
| MMP 3 | Multiplex | Logistic-5 PL | 0.9694 | 0.0163-11.81 |
| MMP 7 | Multiplex | Logistic-5 PL | 0.6031 | 0.1019-76.59 |
| MMP 8 | Multiplex | Logistic-5 PL | 0.9069 | 0.0832-69.90 |
| MMP 9 | Multiplex | Logistic-5 PL | 0.6494 | 0.0602-44.16 |
| MMP 12 | Multiplex | Logistic-5 PL | 0.6298 | 0.0151-11.19 |
| MMP 13 | Multiplex | Logistic-5 PL | 0.44 | 0.0851-62.97 |
| TIMP 1 | ELISA | Linear | r = 0.9998 | 0-50 |
| TIMP 2 | ELISA | Linear | r = 0.9973 | 0-128 |
| TIMP 4 | ELISA | Linear | r = 0.9994 | 0-5 |
| PINP | RIA | Semi Log | r = 0.9935 | 0-250 |
| PIIINP | RIA | Semi Log | r = 0.9974 | 3.019-100 |
| CITP | ELISA | Semi Log | r = 0.9934 | 0-50 |
| CARDIO | ELISA | Linear | r = 0.9997 | 0-0.2 |
| sRage | ELISA | Linear | r = 0.9994 | 0-5 |
| Osteopontin | ELISA | Linear | r = 0.9936 | 0-20 |

2. Demographics

The demographic and clinical data for the 3 groups of subjects is presented in Table 31. The age in the normal referent subjects was slightly younger. However, gender distribution was well balanced between the 3 groups. Body surface area and arterial pressures were higher in the LVH group, and the six minute walk distance was reduced.

3. LV Function

The LV geometry and function data, determined by echocardiography (using the ASE committee recommendations; Lange et al, JASE 2005) are shown in Tables 11 and 12. LV posterior wall thickness and mass were increased in the LVH group, consistent with the classification criteria. LV ejection fraction was slightly but significantly increased in the LVH group. Indices of LV diastolic function, such as atrial filling times or myocardial relaxation (E:A, E′) were prolonged, and estimates of pulmonary capillary wedge pressure were increased in the LVH group. Thus, LV ejection fraction was preserved (systolic function) and indices of diastolic function were impaired in the LVH group. These functional characteristics in accordance with the target pathophysiology: heart failure secondary to primary diastolic dysfunction.

4. Biomarkers

Tables 13 and 14 provides the absolute values for the biomarkers examined. While the sample sizes are somewhat small in this archived data set, some striking and significant differences were observed. MMP-7 appeared increased in both the HTN and LVH groups. In the LVH group, MMP-8 and MMP-9 increased. TIMP-1, -2, and -4 levels were increased in the LVH group. This is the first time that TIMP-2 has been measured in this context, and the fact that TIMP-2 increased by nearly 2-fold in the LVH group is a new finding. With respect to collagen propeptides, the propeptide for collagen III (PIINP) was increased in the LVH group. With respect to biologically active signaling molecules that can influence myocardial fibrosis, cardiotrophin and sRAGE appeared reduced in the LVH group, whereas osteopontin was increased.

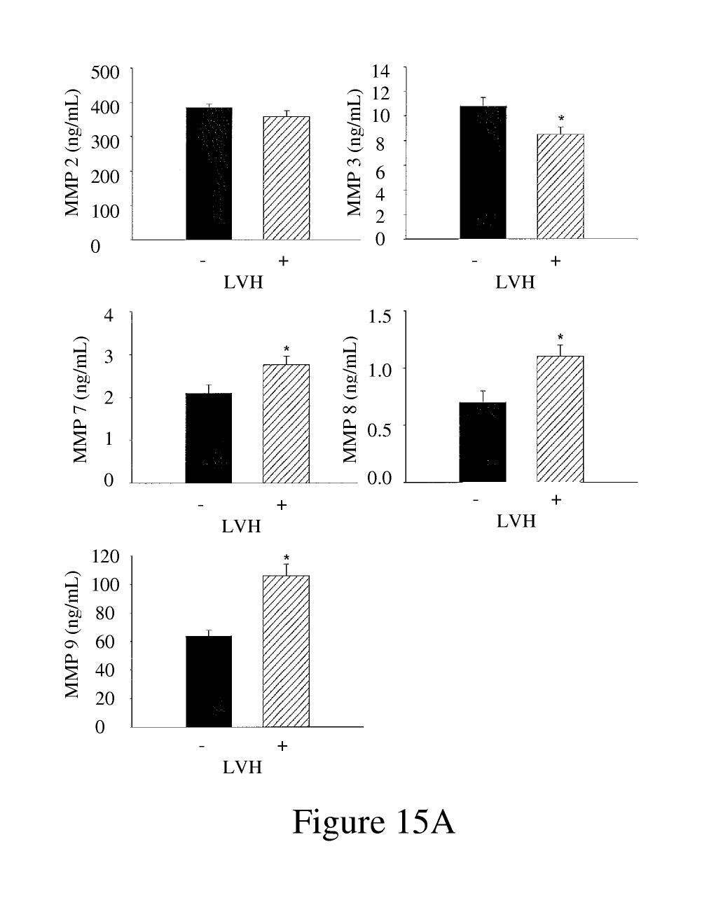

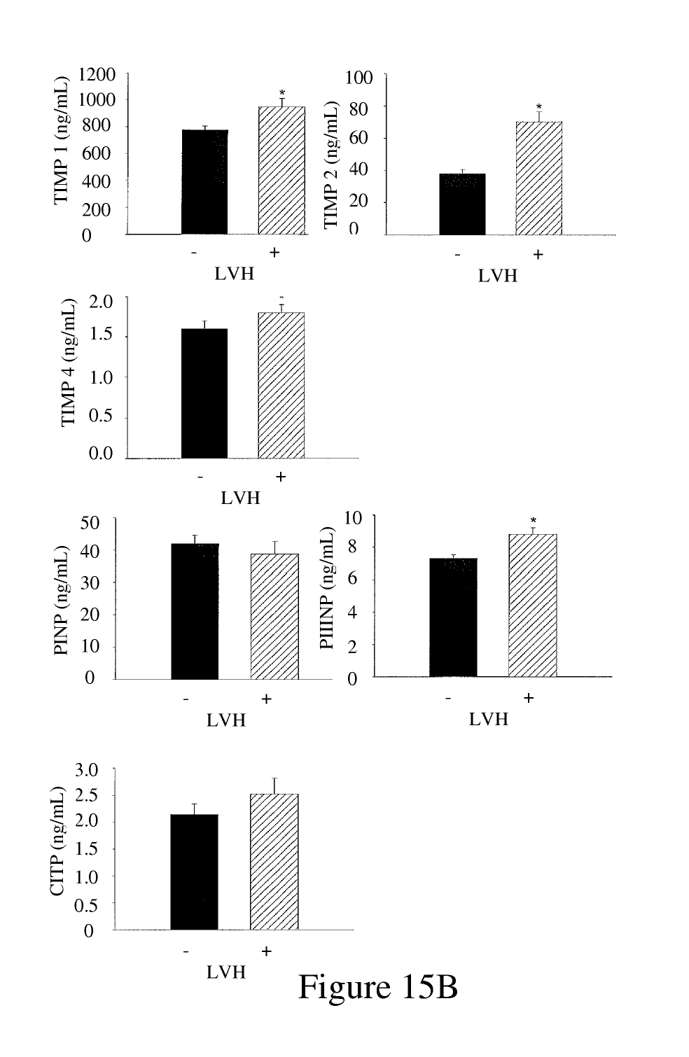

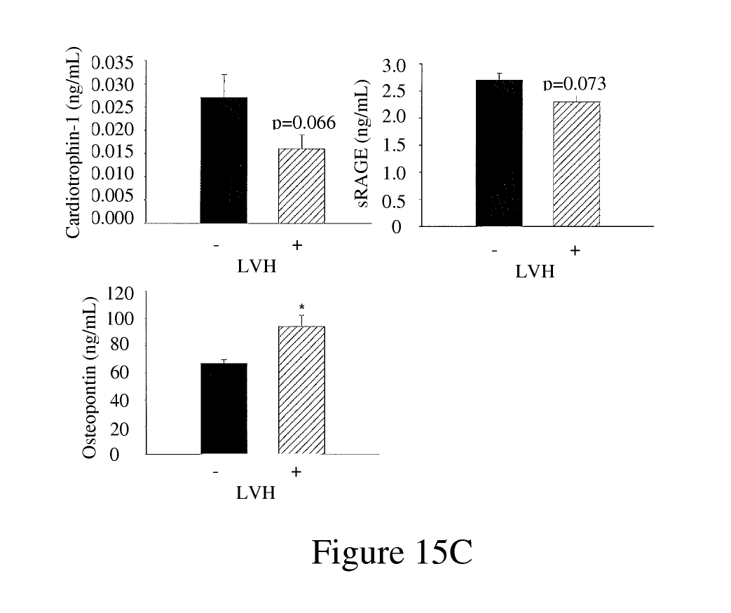

When this data set was dichotomized into those subjects with no LVH and those with LVH, in other words pooling the HTN patients with the normal subjects, some important differences in biomarker profiles were observed (Table 20). In this analysis, substantial changes in TIMP-1, -2 and -4 levels were observed in the LVH group. PIIINP was significantly higher in the LVH group, and cardiotrophin and sRAGE were reduced. Osteopontin levels were approximately 30% higher in the LVH group. A set of histograms highlighting the unique differences in biomarker profiles for these groups is shown in FIG. 15.

The next analysis was performed exclusively on the LVH patients. This group was dichotomized in accordance with CHF symptoms. The CHF classification was based upon NY Heart Association Class, Living with Heart Failure Questionnaire, Six Minute Walk results, and physical exam. The biomarker results for this analysis are shown in Table 21. With respect to MMPs, MMP-2 and MMP-7 appeared higher in those patients with CHF. TIMP-1 and TIMP-4 levels were higher, but strikingly lower TIMP-2 levels were observed in the CHF group. PIINP levels were higher, and CITP levels trended higher in the CHF group. In the CHF group, sRAGE levels were higher.

An initial correlation matrix for the key response variable; LV mass to biomarker profiles is presented in Table 22. Significant correlative values were observed for MMP-8, MMP-9, and all TIMPs. Interestingly, a negative correlation was observed for PINP, and a positive correlation for PIINP and CITP was observed. Thus, a strong positive correlation was observed between LV mass and the PIINP/PINP ratio. A positive correlation was observed for osteopontin, and a negative correlation for sRAGE was obtained.

5. Interpretation

In this archived group of subjects, a complete cassette of biomarkers were examined and related to the presence and absence of LVH, and the presence and absence of CHF. With respect to LVH, a unique profile of MMPs appeared to be elevated (MMP-7, -8, -9). These MMPs are primarily synthesized and release by resident cells such as macrophages and mast cells. Thus, the emergence of these MMP types is indicative of a local induction of a myocardial matrix turnover process, which is promulgated through activation and expansion of a macrophage type cell. It should be noted that the elevated levels of MMP-8 which were observed in the LVH group, are 5-fold lower than those reported in patients following an acute myocardial injury such as myocardial infarction (Webb, Circulation, 2006; 114). This MMP profile can be an early indicator of patients with HTN, evolving into LVH as indicated by the univariate correlation analysis.

With respect to TIMPs, these MMP inhibitors continue to demonstrate a strong relationship with LVH and the presence of CHF. Notably, while TIMP-1, -2, and -4 levels were elevated in LVH patients, subgroup analysis indicated that TIMP-2 is actually lower. This indicates that a subset of TIMPs can identify patients with ongoing and developing LVH, and a second subset of TIMPs can identify those with increased risk of CHF.

This is one of the largest studies to examine collagen propeptides and telopeptides in a group with LVH and the presence and absence of CHF. PIIINP was increased in the LVH group which is indicative of increased synthesis of collagen type III. While this index of collagen synthesis was increased, a marker of collagen degradation (CITP) was unchanged. Thus, taking these observations coupled with the TIMP levels, would clearly indicate a state where increased collagen accumulation would occur.

Cardiotrophin is a member of the IL-6 family of cytokines, and some studies have identified increased levels following myocardial infarction (MI) in animals and patients. Cardiotrophin appears to be synthesized primarily by the myocardium and a clear, and robust signal was observed in normal subjects. While initially considered to be a pro-fibrotic signaling molecule, this functionality has been called into question (Freed, C V Research, 2005; 65). Specifically, it appears that cardiotrophin can prevent myofibroblast transformation and the increased synthesis of fibrillar collagen. In that sense, cardiotrophin can serve as an “antagonist” to profibrotic signals such as angiotensin II and transforming growth factor beta. Thus, the reduction of cardiotrophin in the LVH group which was not observed in the HTN group, indicates that a key local regulatory protein which would prevent myofibroblast transformation and collagen synthesis has been lost. This is thus a novel marker with respect to identifying the “transition” from HTN to LVH.

There was little known about the normal reference values for the soluble receptor for advanced glycated end products (sRAGE) and this disclosure provides the first index values for this biomarker. What is of particular interest is that in the subgroup analysis of LVH patients with and without CHF, sRAGE levels were increased. This is indicative of increased activation of the AGE receptor. Osteopontin levels were significantly increased in the LVH group. Since osteopontin belongs to the family of matrikines that favor fibroblast collagen synthesis, and matrix formation, then this observation is consistent with the pathophysiology of LVH. There have been few studies that have measured osteopontin, and none have measured this biomarker in a robust group of normal and LVH subjects. Moreover, the fact that osteopontin was not further increased in the CHF sub-analysis indicates that this is another biomarker for identifying the transition to LVH.

6. Summary

A unique portfolio of biomarkers emerged from this example. Specifically, in 150 archived subjects, a specific MMP/TIMP signature emerged which was associated with LVH. Moreover, cardiotrophin and osteopontin emerged as biomarkers which specifically changed in the transition to LVH. With respect to CHF relationships, a unique shift in MMP-2, MMP-7, TIMP-2, sRAGE, and CITP levels occurred. This indicates that significant changes in matrix structure and function have begun to occur, which was associated with worsening of the underlying LV diastolic dysfunction.

### E. Example 6

### Sub-Group Analysis of Biomarkers as Indicators of Left Ventricular Remodeling and Congestive Heart Failure

1. Background

A significant area of development is that of “personalized medicine” which can be defined as tailoring treatment strategies and titrating pharmacological agents to biological endpoints that are specific for each patient. Currently, treatments and pharmacological compounds are administered on a “one size fits all” theory. However, this approach can result in certain patient populations experiencing increased number of adverse events which can be due to failure of meeting a therapeutic index, or by exceeding a therapeutic index. Thus, a more personalized approach for treatment strategies has been identified as a top priority at the FDA and in industry. Accordingly, intense interest has been generated in the domain of “biomarkers” which can provide a more rapid, and streamlined approach for assessing pharmacological efficacy and for more individualized dose titration. This is certainly a relevant issue when it comes to gender and ethnicity issues and cardiovascular medicine. For example, significant interactions exist with respect to gender and a cardiovascular response to a given stress (such as hypertension or ischemic events). Moreover, it has been clearly established that the cardiovascular response to hypertension and anti-hypertensive medications have a differential response in certain ethnicities such as Blacks.

This example examines, for the first time, ethnicity and gender relationships with respect to the progression of hypertensive heart disease and the MMP/TIMP biomarker matrix. The results from this analysis identified that a unique MMP/TIMP signature occurred with respect to gender and ethnicity in patients with hypertensive heart disease. Moreover, the results indicate that both gender and ethnicity are independent variables which influence the MMP/TIMP profile indicating that these can be considered in the diagnostic matrix when using MMP/TIMPs as biomarkers of diagnosis/prognosis/treatment efficacy.

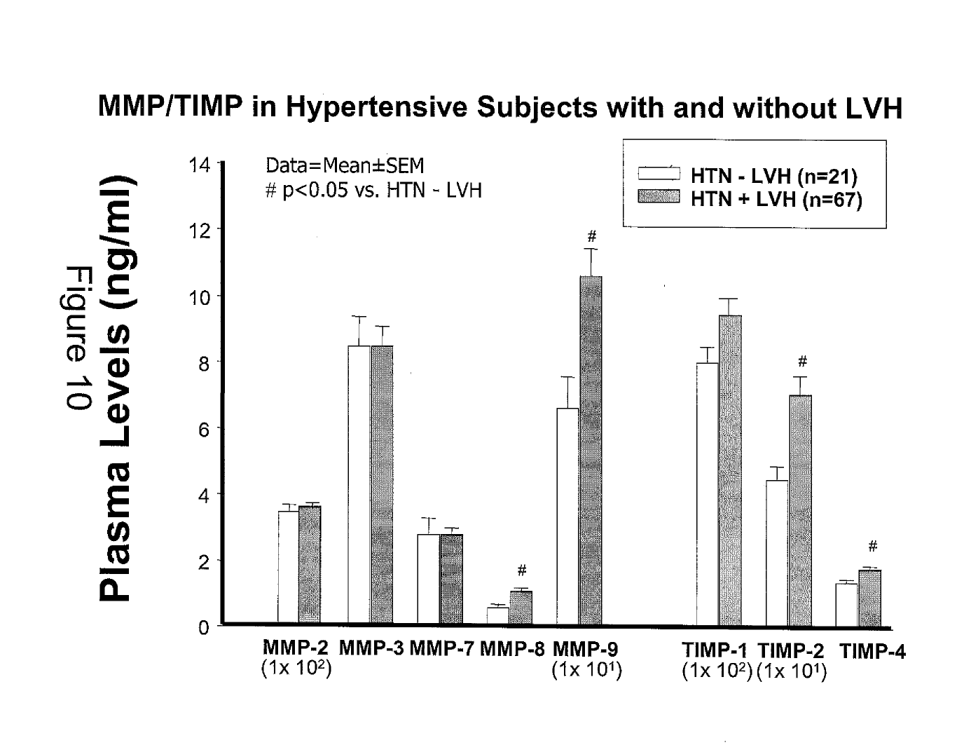

This example is a continuation of work on biomarker profiling as indicators of LVH and CHF. During the conduct of this work, sub-group analysis was performed. Specifically, sub-group analysis was performed in the patient database with respect to differences in MMP/TIMP profiles across gender, ethnicity, family history, and medication profiles. The summary of results, which formed the basis for the initial findings, are shown in FIG. 10. This example is a result of this analysis and describes the preliminary results where a subgroup analysis was performed as shown in FIG. 11. The results of this analysis demonstrated that a differential profile in MMP-3 and MMP-7 occurred in male patients with hypertensive heart disease when compared to females (FIG. 12). Since MMP-3 and MMP-7 constitute a sub-class of MMPs, called the “lysins”, then it is possible that a gender specific regulation of these MMP types occurs in patients with hypertensive heart disease. The next analysis demonstrated that a differential level of TIMP-2 was observed between Black and White patients (FIG. 13). Since there are 4 TIMPs, all with different biological functions, then this indicates that different regulatory pathways can be operative with respect to the pathogenesis of hypertensive heart disease and ethnicity.

A multiplex approach was utilized to measure all of the disclosed. The pattern of biomarkers measured in this example with respect to the MMP/TIMP profiles and relation to gender and ethnicity are unique. This results indicate that another variable or layer can be added to this biomarker matrix which involves gender and ethnicity specific variables. Thus, gender and/or ethnicity can be added into the disclosed diagnostic pathway to provide more patient specific prediction and diagnosis.

Patients with uncontrolled or poorly managed hypertension can exhibit changes in their myocardial extracellular matrix (ECM), specifically collagen homeostasis. This can lead to Left Ventricular Hypertrophy (LVH). These changes can also be reflected in the balance between the ECM proteases—matrix metalloproteinases (MMPs) and the tissue inhibitors of matrix metalloprtoeinases (TIMPs). Furthermore, changes in these metalloproteinases have a significant role in the structural, functional, and clinical outcomes of hypertensive heart disease. Additionally, hypertension affects various populations differently. However, a MMP and TIMP profile has yet to be clearly established in regard to gender and ethnicity.

2. Methods

LV Mass, plasma MMP-2,3,7,8, and 9, TIMP 1, 2, and 4 values, and Doppler echocardiography were acquired from 150 subjects who were carefully screened and enrolled in the study after providing informed consent. These subjects were further bifurcated into a (1) reference control group, with no evidence of hypertension (2) Hypertensive subject group. Subjects with hypertension were further grouped into those with the absence or presence of LVH. Lastly, subjects who were found to have hypertension and LVH were stratified based on gender (male or female) and ethnicity (black or white).

i. Calibration

The Bio-Plex 200 array reader (Cat# 171-000205, BioRad, CA) was calibrated before each assay using a commercially available calibration kit (Cat#. 171-203060, BioRad, CA). Calibration is the process that utilizes microspheres (or calibrators) with a constant fluorescent intensity to adjust gain settings in the Bio-Plex 200 array reader's detector for optimal and consistent microsphere classification and reporter readings. The calibrators (CAL1 and CAL2 beads) are microspheres with stable fluorescent intensities in the RP1, CL1, CL2 wavelength ranges. Briefly, the CAL1 beads calibrate the array reader's doublet discriminator and classification channels, while the CAL2 beads calibrate the array reader's reporter channel for reporter fluorescence detection.

ii. Validation

The array reader is validated using BioRad's validation kit (Cat# 171-203001, BioRad, CA). This process evaluates the array reader's optical alignment, reporter channel performance, efficiency of multiplexing, and integrity of fluidics. This procedure can also be used to discriminate between assay and instrumentation problems.

iii. Software

BioPlex Manager Software 4.1

3. Results

Compared with reference control, patients with hypertension had significant changes in MMP/TIMP values. Subjects with hypertension and hypertrophy exhibited a differential profile when compared to hypertensive patients with no hypertrophy. Males and females, when compared also showed significant changes in MMP/TIMP values as well as blacks versus white subjects.

4. Conclusion

There is a unique MMP/TIMP profile that exists in Hypertension and a gender and ethnicity sub-profile may exist. This example indicates that there are specific plasma profiles of biomarkers, specifically the MMPs and TIMPs, in the myocardial matrix that can play a role in the clinical manifestations of hypertensive heart disease.

### F. Example 7

### Multivariate Analysis of Biomarkers as Indicators of Left Ventricular Remodeling and Congestive Heart Failure

1. Overview

As described and discussed elsewhere herein, univariate analysis was performed on all of the biomarkers measured in a cohort of subjects. This original cohort was also used to examine multivariable relations of the biomarkers to major outcomes measures. This analysis was focused on the development of two fundamental diagnostic tests/criteria:

1. A screening test to identify patients with unrecognized hypertensive heart disease as defined as an increase in LV mass (a measure of LV hypertrophy).

2. A companion diagnostic test that could be utilized to assess the underlying progression of hypertensive heart disease in patients identified to be at risk—i.e., identified to have LV hypertrophy. For this approach, indices of clinical heart failure were utilized which included pulmonary capillary wedge pressure estimates (PCWP), six minute walk test, and the presence and absence of congestive heart failure (CHF).

2. Methods

Statistical analyses were performed with SAS (version 9.1; SAS Institute, Inc, Cary, N.C.). Forms of the relationship between outcome and important predictors were explored using generalized additive models (which do not necessarily assume linear relationships). Where linear relationships were deemed satisfactory, multivariable linear regression was used to analyze continuous outcomes: left ventricular mass, PCWP, EDV mass ratio, and six minute walk. Normality of outcomes was assessed and log transformations were applied where appropriate. Multivariable logistic regression was used to analyze indicators of PCWP (>14 versus <=15) and Heart failure. Initially, in the model building procedure, independent variables were assessed for univariate associations with outcomes and those with p<0.20 were retained in multivariable analyses. In subsequent multivariable analyses, independent variables with p-value>0.05 were removed from the model in a stepwise fashion. Gender interactions with MMPs were assessed in all analyses, and MMP variables found to interact with gender were centered to avoid multicollinearity. Adjusted R2 and C statistic values, which reflect the predictive value of the models, are reported for linear and logistic regression models, respectively. Linear regression betas and p-values or logistic regression odds ratios [95% Confidence Interval] are presented.

3. Results

1. LV mass. NTBNP and the expected MMP/TIMP indices (MMP-2, MMP-9, MMP-9/TIMP-1) clearly come out in a multivariable analysis and the interaction with gender is important. What did not fall out of this analysis was the matrikines or telopetides that showed significance in the univariate analysis. Nevertheless, there are 5 strong biomarkers that can be used together to provide a reasonable prediction of LV mass (correlation ˜0.7 or higher). As seen in Table 26, gender interaction effects imply that the effect of MMP2 on increased LV mass is significantly greater for males than for females. The effect of MMP8 on increased LV mass is significantly greater for females than for males.

As seen in Table 27, predictors are similar to the strongest predictors of LV mass although MMP-8 and -9, the interaction between gender and MMP-8, and the MMP-9/TIMP-1 ratio fall out of the model. It therefore might be expected that expression of these MMPs is actually correlated with BMI. As in the LV mass analysis, there is still a significant MMP-2 and gender interaction. Note that the main effect of gender is in the opposite direction when LV mass index is considered. However, the main effect, which means nothing in the presence of an interaction, is not important here. The interaction demonstrates that the effect of MMP-2 on increased LV mass index is significantly greater for males than for females. There appears to be a stronger correlation between MMP2 and LV mass Index for males than females. This interaction could be driven by the males at the high end of the spectrum for LV mass, and not by those with more normal LV values.

2. With respect to PCWP, the collagen telopeptide PIINP was incorporated into the multivariable model. Interestingly, an entirely different portfolio of MMPs emerged here: MMP-3, -7,)-8 (Table 28). With respect to the six minute walk, MMP-2, MMP-8, MMP-9, and the MMP-9/TIMP-1 ratio emerged as indicators (Table 29). With respect to congestive heart failure (CHF), a dichotomous odds-ratio was performed and identified MMP-7, MMP-9, and the MMP-9/TIMP-1 ratio in the model (Table 30).

These results indicate that a unique cassette of markers can be utilized for screening for LV hypertrophy, and another cassette of markers can be utilized for identifying the progression of LVH to CHF.

TABLE 11
Demographics and Arterial Pressures; Archived Subjects

| | Normals | HTN | LVH | F value | p value |
|---|---|---|---|---|---|
| Age (years) | 51.5 ± 1.6 | 58.1 ± 2.0 | 59.6 ± 1.5* | 7.4 | <0.001 |
| Gender (F/M) | 36/26 | 12/9 | 38/29 | n/a | n/a |
| Body Surface Area | 1.88 ± 0.03 | 1.93 ± 0.05 | 2.11 ± 0.03*# | 14.3 | <0.001 |
| (m2) | | | | | |
| Heart Rate (bpm) | 68 ± 1 | 69 ± 2 | 69 ± 1 | 0.1 | 0.876 |
| Arterial Systolic | 125 ± 2 | 133 ± 3 | 141 ± 2* | 15.6 | <0.001 |
| Pressure (mmHg) | | | | | |
| Arterial Diastolic | 75 ± 1 | 78 ± 2 | 79 ± 1* | 3.1 | 0.049 |
| Pressure (mmHg) | | | | | |
| Six minute walk (m) | 394 ± 44 | 434 ± 56 | 284 ± 20*# | 5.6 | 0.006 |
| Sample Size | 62 | 21 | 67 | | |

Values presented as Mean ± SEM.
*p < 0.05 compared with reference controls.
#p < 0.05 compared with HTN.
All data analyzed using One-Way ANOVA followed by pair-wise comparison using Bonferroni method.

TABLE 12
Demographics and Arterial Pressures; Archived Subjects

| | Normals | HTN | LVH w/o CHF | LVH w/CHF |
|---|---|---|---|---|
| Age (years) | 51.5 ± 1.6 | 58.1 ± 2.0 | 55.8 ± 1.7 | 64.0 ± 2.5*† |
| Gender (F/M) | 36/26 | 12/9 | 20/16 | 18/13 |
| Body Surface Area (m2) | 1.88 ± 0.03 | 1.93 ± 0.05 | 2.12 ± 0.04 | 2.10 ± 0.05 |
| Heart Rate (bpm) | 68 ± 1 | 69 ± 2 | 70 ± 2 | 69 ± 2 |
| Arterial Systolic Pressure | 125 ± 2 | 133 ± 3 | 140 ± 2* | 143 ± 4* |
| (mmHg) | | | | |
| Arterial Diastolic Pressure | 75 ± 1 | 78 ± 2 | 82 ± 2* | 76 ± 2 |
| (mmHg) | | | | |
| Six minute walk (m) | 394 ± 44 | 434 ± 56 | 327 ± 25 | 231 ± 28*# |
| Sample Size | 62 | 21 | 36 | 31 |

Values presented as Mean ± SEM.
*p < 0.05 compared with reference controls.
#p < 0.05 compared with HTN.
†p < 0.05 compared with LVH w/o CHF.
All data analyzed using One-Way ANOVA followed by pair-wise comparison using Bonferroni method.

TABLE 13
Echocardiographic parameters; Archived Subjects

| | Normals | HTN | LVH | F value | p value |
|---|---|---|---|---|---|
| LV End Diastolic | 4.7 ± 0.05 | 4.6 ± 0.1 | 4.7 ± 0.07 | 0.84 | 0.43 |
| Dimension (cm) | | | | | |
| End Diastolic LV | 0.93 ± 0.02 | 0.95 ± 0.02 | 1.36 ± 0.02*# | 136.05 | <0.001 |
| Posterior Wall | | | | | |
| Thickness (cm) | | | | | |
| LV Fractional | 0.38 ± 0.01 | 0.38 ± 0.01 | 0.40 ± 0.01* | 3.72 | 0.03 |
| Shortening (%) | | | | | |
| LV End Diastolic | 101.8 ± 2.6 | 95.8 ± 3.8 | 103.4 ± 3.6 | 0.76 | 0.47 |
| Volume (mL) | | | | | |
| LV End Diastolic | 54.4 ± 1.4 | 49.4 ± 1.8 | 49.6 ± 1.8 | 2.80 | 0.06 |
| Volume Indexed | | | (*p = 0.086) | | |
| (mL/m2) | | | | | |
| LV Ejection Fraction | 66.6 ± 0.6 | 67.2 ± 1.2 | 70.1 ± 1.1* | 4.14 | 0.02 |
| (%) | | | | | |
| LV Mass (g) | 153.1 ± 4.5 | 148.0 ± 6.0 | 263.7 ± 7.8*# | 95.62 | <0.001 |
| LV Mass Indexed | 81.1 ± 1.7 | 76.9 ± 3.0 | 126.0 ± 3.5*# | 83.61 | <0.001 |
| (g/m2) | | | | | |
| Volume:Mass Ratio | 0.68 ± 0.02 | 0.66 ± 0.02 | 0.40 ± 0.01*# | 44.89 | <0.001 |
| (mL/g) | | | | | |
| E:A Ratio | 1.16 ± 0.05 | 0.91 ± .0.04* | 0.92 ± 0.04* | 8.73 | <0.001 |
| E:E′ Ratio | 5.9 ± 0.2 | 6.8 ± 0.5 | 9.2 ± 0.5*# | 21.44 | <0.001 |
| PCWP (mmHg) | 9.7 ± 0.3 | 10.8 ± 0.6 | 14.0 ± 0.60*# | 21.44 | <0.001 |
| Sample Size | 62 | 21 | 67 | | |

Values presented as Mean ± SEM.
*p < 0.05 compared with reference controls.
#p < 0.05 compared with HTN.
All data analyzed using One-Way ANOVA followed by pair-wise comparison using Bonferroni method.

TABLE 14
Echocardiographic parameters; Archived Subjects

| | Normals | HTN | LVH w/o CHF | LVH w/ CHF |
|---|---|---|---|---|
| LV End Diastolic | 4.7 ± 0.05 | 4.6 ± 0.1 | 4.7 ± 0.1 | 4.7 ± 0.1 |
| Dimension (cm) | | | | |
| End Diastolic LV Posterior | 0.92 ± 0.02 | 0.95 ± 0.02 | 1.36 ± 0.03*# | 1.37 ± 0.03*# |
| Wall Thickness (cm) | | | | |
| LV Fractional Shortening | 0.38 ± 0.01 | 0.38 ± 0.01 | 0.39 ± 0.01 | 0.41 ± 0.01* |
| (%) | | | | |
| LV End Diastolic Volume | 101.8 ± 2.6 | 95.8 ± 3.8 | 98.8 ± 4.6 | 108.7 ± 5.6 |
| (mL) | | | | |
| LV End Diastolic Volume | 54.4 ± 1.4 | 49.4 ± 1.8 | 47.3 ± 2.4* | 52.2 ± 2.7 |
| Indexed (mL/m2) | | | | |
| LV Ejection Fraction (%) | 66.6 ± 0.6 | 67.2 ± 1.2 | 68.8 ± 1.3 | 71.6 ± 1.8* |
| LV Mass (g) | 153.1 ± 4.5 | 148.0 ± 6.0 | 261.2 ± 11.7*# | 266.5 ± 10.2*# |
| LV Mass Indexed (g/m2) | 81.1 ± 1.7 | 76.9 ± 3.0 | 123.9 ± 4.8*# | 128.3 ± 5.1*# |
| Volume:Mass Ratio (mL/g) | 0.68 ± 0.02 | 0.66 ± 0.02 | 0.39 ± 0.02*# | 0.41 ± 0.2*# |
| E:A Ratio | 1.16 ± 0.05 | 0.91 ± .0.04* | 0.88 ± 0.04* | 0.97 ± 0.06 |
| | | | | (*p = 0.083) |
| E:E′ Ratio | 5.9 ± 0.2 | 6.8 ± 0.5 | 7.6 ± 0.3* | 11.6 ± 0.8*#† |
| PCWP (mmHg) | 9.7 ± 0.3 | 10.8 ± 0.6 | 11.9 ± 0.5* | 17.1 ± 1.1*#† |
| Sample Size | 62 | 21 | 36 | 31 |

Values presented as Mean ± SEM.
*p < 0.05 compared with reference controls.
#p < 0.05 compared with HTN.
†p < 0.05 compared with LVH w/o CHF.
All data analyzed using One-Way ANOVA followed by pair-wise comparison using Bonferroni method.

TABLE 15
Biomarker parameters; Archived Patients

| | Normals | HTN | LVH | F value | p value |
|---|---|---|---|---|---|
| MMP 2 (ng/ml) | 396.0 ± 12.4 | 347.1 ± 21.0 | 358.1 ± 16.7 | 2.19 | 0.115 |
| MMP 3 (ng/ml) | 11.6 ± 0.9 | 8.5 ± 0.9 | 8.5 ± 0.6* | 4.92 | 0.009 |
| MMP 7 (ng/ml) | 1.9 ± 0.1 | 2.8 ± 0.5 | 2.8 ± 0.2* | 5.33 | 0.006 |
| | | (*p = 0.081) | | | |
| MMP 8 (ng/ml) | 0.79 ± 0.1 | 0.61 ± 0.07 | 1.06 ± 0.1*# | 6.44 | 0.002 |
| MMP 9 (ng/ml) | 62.9 ± 4.3 | 66.3 ± 9.6 | 106.3 ± 8.1*# | 12.23 | <0.001 |
| TIMP-1 (ng/ml) | 769.1 ± 34.5 | 802.4 ± 48.2 | 947.3 ± 63.6* | 3.42 | 0.035 |
| TIMP-2 (ng/ml) | 35.7 ± 2.9 | 44.9 ± 4.20 | 70.2 ± 6.1*# | 14.12 | <0.001 |
| TIMP-4 (ng/ml) | 1.63 ± 0.06 | 1.40 ± 0.11 | 1.80 ± 0.10# | 3.21 | 0.043 |
| PINP (ng/ml) | 44.1 ± 2.9 | 34.8 ± 5.8 | 38.7 ± 4.0 | 1.01 | 0.368 |
| PIIINP (ng/ml) | 7.3 ± 0.2 | 7.4 ± 0.6 | 8.8 ± 0.4* | 5.26 | 0.006 |
| CITP (ng/ml) | 2.2 ± 0.2 | 2.0 ± 0.4 | 2.5 ± 0.3 | 0.87 | 0.421 |
| Cardiotrophin-1 | 23.8 ± 3.8 | 38.9 ± 18.7 | 16.1 ± 3.1 | 2.73 | 0.069 |
| (ng/ml) * 10-3 | | | (*p = 0.078) | | |
| sRAGE (ng/ml) | 2.8 ± 0.1 | 2.1 ± 0.2 | 2.3 ± 0.2* | 3.85 | 0.023 |
| Osteopontin | 67.1 ± 3.5 | 66.4 ± 3.8 | 94.0 ± 8.3* | 5.42 | 0.005 |
| (ng/ml) | | | | | |
| NTBNP (pg/mL) | 91.4 ± 12.7 | 92.4 ± 28.8 | 199.1 ± 32.6* | 5.53 | 0.005 |
| Troponin (ng/mL) * 10-3 | 0.89 ± 0.64 | 1.25 ± 0.99 | 1.41 ± 0.49 | 0.23 | 0.796 |
| PIIINP:PINP Ratio | 0.22 ± 0.02 | 0.28 ± 0.04 | 0.30 ± 0.02* | 3.90 | 0.022 |
| PINP:CITP Ratio | 30.6 ± 4.3 | 53.6 ± 19.4 | 38.0 ± 6.3 | 1.50 | 0.227 |
| PIIINP:CITP | 4.9 ± 0.5 | 11.4 ± 4.0* | 7.8 ± 0.9 | 4.92 | 0.009 |
| Ratio | | | | | |
| Sample Size | 62 | 21 | 67 | | |

Values presented as Mean ± SEM.
*p < 0.05 compared with reference controls.
#p < 0.05 compared with HTN.
All data analyzed using One-Way ANOVA followed by pair-wise comparison using Bonferroni method.

TABLE 16
Biomarker parameters; Archived Patients

| | Normals | HTN | LVH w/o CHF | LVH w/ CHF |
|---|---|---|---|---|
| MMP 2 (ng/ml) | 396.0 ± 12.4 | 347.1 ± 21.0 | 330.2 ± 20.9* | 390.4 ± 26.0 |
| MMP 3 (ng/ml) | 11.6 ± 0.9 | 8.5 ± 0.9 | 7.9 ± 0.8* | 9.1 ± 0.8 |
| MMP 7 (ng/ml) | 1.9 ± 0.1 | 2.8 ± 0.5 | 2.2 ± 0.3 | 3.4 ± 0.4*† |
| MMP 8 (ng/ml) | 0.79 ± 0.1 | 0.61 ± 0.07 | 1.1 ± 0.1 | 1.0 ± 0.1# |
| | | | (*p = 0.065)# | |
| MMP 9 (ng/ml) | 62.9 ± 4.3 | 66.3 ± 9.6 | 115.4 ± 12.9*# | 95.8 ± 8.8* |
| TIMP-1 (ng/ml) | 769.1 ± 34.5 | 802.4 ± 48.2 | 580.6 ± 45.5*# | 1373.1 ± 72.0*#† |
| TIMP-2 (ng/ml) | 35.7 ± 2.9 | 44.9 ± 4.20 | 80.0 ± 9.1*# | 58.4 ± 7.3* |
| TIMP-4 (ng/ml) | 1.63 ± 0.06 | 1.40 ± 0.11 | 1.48 ± 0.09 | 2.18 ± 0.16*#† |
| PINP (ng/ml) | 44.1 ± 2.9 | 34.8 ± 5.8 | 38.2 ± 6.3 | 39.2 ± 4.8 |
| PIIINP (ng/ml) | 7.3 ± 0.2 | 7.4 ± 0.6 | 8.1 ± 0.4 | 9.6 ± 0.7*# |
| | | | | (†p = 0.095) |
| CITP (ng/ml) | 2.2 ± 0.2 | 2.0 ± 0.4 | 2.1 ± 0.3 | 3.0 ± 0.5 |
| Cardiotrophin-1 (ng/ml) * 10−3 | 23.8 ± 3.8 | 38.9 ± 18.7 | 14.7 ± 4.2 | 16.5 ± 4.8 |
| sRAGE (ng/ml) | 2.8 ± 0.1 | 2.1 ± 0.2 | 1.9 ± 0.2* | 2.6 ± 0.3 |
| Osteopontin (ng/ml) | 67.1 ± 3.5 | 66.4 ± 3.8 | 99.5 ± 13.8* | 87.8 ± 8.6 |
| NTBNP(pg/mL) | 91.4 ± 12.7 | 92.4 ± 28.8 | 157.3 ± 35.5 | 248.0 ± 56.4*# |
| Troponin(ng/mL) * 10−3 | 0.89 ± 0.64 | 1.25 ± 0.99 | 1.71 ± 0.86 | 1.10 ± 0.42 |
| PIIINP:PINP Ratio | 0.22 ± 0.02 | 0.28 ± 0.04 | 0.29 ± 0.03 | 0.30 ± 0.03 |
| PINP:CITP Ratio | 30.0 ± 4.4 | 53.6 ± 19.4 | 42.4 ± 10.2 | 34.2 ± 6.8 |
| PIIINP:CITP Ratio | 4.9 ± 0.5 | 11.4 ± 4.0* | 7.4 ± 1.0 | 8.3 ± 1.6 |
| Sample Size | 62 | 21 | 36 | 31 |

Values presented as Mean ± SEM.
*p < 0.05 compared with reference controls.
#p < 0.05 compared with HTN.
†p < 0.05 compared with LVH w/o CHF. All data analyzed using One-Way ANOVA followed by pair-wise comparison using Bonferroni method.

TABLE 17
Proportion of Troponin Equal or Greater than 0.001 (ng/mL)

| | Less Than | Equal or Greater than |
|---|---|---|
| | 0.001 ng/ml | 0.001 ng/mL |
| Normals (%) | 86.8 | 13.2 |
| HTN(%) | 75.0 | 25.0 |
| LVH (%) | 75.0 | 25.0 |

Sample Size (n = 62 Normals, n = 21 HTN, n = 67 LVH). Values presented as proportion. X2 = 2.74 and p = 0.25.

TABLE 18
Frequency of Troponin Equal or Greater Than 0.001 (ng/mL)

| | Less Than | Equal or Greater than |
|---|---|---|
| | 0.001 ng/ml | 0.001 ng/mL |
| Normals (n) | 46 | 7 |
| HTN(n) | 12 | 4 |
| LVH (n) | 48 | 16 |

Sample Size (n = 62 Normals, n = 21 HTN, n = 67 LVH). Values presented as frequency.

TABLE 19
Troponin and HTN; Archived Patients

| | Normals | HTN |
|---|---|---|
| Troponin (ng/ml) * 10−3 | 0.89 ± 0.64 | 1.38 ± 0.44 |
| Sample Size | 54 | 81 |

Values presented as Mean ± SEM.
*p < 0.05 compared with Normals.
All data analyzed using two sample t-test.

TABLE 20
Biomarker parameters and LVH; Archived Patients

| | No LVH | LVH |
|---|---|---|
| MMP 2 (ng/ml) | 384.2 ± 10.9 | 358.1 ± 16.7 |
| MMP 3 (ng/ml) | 10.8 ± 0.7 | 8.5 ± 0.6* |
| MMP 7 (ng/ml) | 2.1 ± 0.2 | 2.8 ± 0.2* |
| MMP 8 (ng/ml) | 0.7 ± 0.1 | 1.1 ± 0.1* |
| MMP 9 (ng/ml) | 63.7 ± 3.9 | 106.3 ± 8.1* |
| TIMP-1 (ng/ml) | 777.5 ± 28.4 | 947.3 ± 63.6* |
| TIMP-2 (ng/ml) | 38.1 ± 2.4 | 70.2 ± 6.1* |
| TIMP-4 (ng/ml) | 1.6 ± 0.1 | 1.8 ± 0.1* |
| PINP (ng/ml) | 41.9 ± 2.7 | 38.7 ± 4.0 |
| PIIINP (ng/ml) | 7.3 ± 0.2 | 8.8 ± 0.4* |
| CITP (ng/ml) | 2.1 ± 0.2 | 2.5 ± 0.3 |
| | | (*p = 0.429) |
| Cardiotrophin-1 (ng/ml) * 10−3 | 27.4 ± 5.3 | 16.1 ± 3.1 |
| | | (*p = 0.066) |
| sRAGE (ng/ml) | 2.7 ± 0.1 | 2.3 ± 0.2 |
| | | (*p = 0.073) |
| Osteopontin (ng/ml) | 66.9 ± 2.8 | 94.0 ± 8.3* |
| PIIINP:PINP Ratio | 0.24 ± 0.02 | 0.30 ± 0.19 |
| PINP:CITP Ratio | 35.9 ± 5.6 | 38.6 ± 6.3 |
| PIIINP:CITP Ratio | 6.4 ± 1.1 | 7.8 ± 0.9 |
| Sample Size | 83 | 67 |

Values presented as Mean ± SEM.
*p < 0.05 compared with reference controls.
All data analyzed using One-Way ANOVA followed by pair-wise comparison using Bonferroni method.

TABLE 21
Biomarker parameters and CHF; Archived Patients

| | No CHF | CHF |
|---|---|---|
| MMP 2 (ng/ml) | 330.2 ± 20.9 | 390.4 ± 26.0 |
| | | (*p = 0.072) |
| MMP 3 (ng/ml) | 7.9 ± 0.8 | 9.1 ± 0.8 |
| MMP 7 (ng/ml) | 2.2 ± 0.3 | 3.4 ± 0.4* |
| MMP 8 (ng/ml) | 1.1 ± 0.1 | 1.0 ± 0.1 |
| MMP 9 (ng/ml) | 115.4 ± 12.9 | 95.8 ± 8.8 |
| TIMP-1 (ng/ml) | 580.6 ± 45.5 | 1373.1 ± 72.0* |
| TIMP-2 (ng/ml) | 80.0 ± 9.1 | 58.4 ± 7.3 |
| | | (*p = 0.078) |
| TIMP-4 (ng/ml) | 1.5 ± 0.1 | 2.2 ± 0.2* |
| PINP (ng/ml) | 38.2 ± 6.3 | 39.2 ± 4.8 |
| PIIINP (ng/ml) | 8.1 ± 0.4 | 9.6 ± 0.7* |
| CITP (ng/ml) | 2.1 ± 0.3 | 3.0 ± 0.5 |
| | | (*p = 0.141) |
| Cardiotrophin-1 (ng/ml) * 10−3 | 14.7 ± 4.2 | 16.5 ± 4.8 |
| sRAGE (ng/ml) | 1.9 ± 0.2 | 2.6 ± 0.3 |
| | | (p = 0.065) |
| Osteopontin (ng/ml) | 99.5 ± 13.8 | 87.8 ± 8.6 |
| PIIINP:PINP Ratio | 0.29 ± 0.03 | 0.30 ± 0.03 |
| PINP:CITP Ratio | 42.4 ± 10.2 | 34.2 ± 6.8 |
| PIIINP:CITP Ratio | 7.4 ± 1.0 | 8.3 ± 1.6 |
| Sample Size | 36 | 31 |

Values presented as Mean ± SEM.
*p < 0.05 compared with reference controls.
All data analyzed using One-Way ANOVA followed by pair-wise comparison using Bonferroni method.

TABLE 22
Correlations for Relations between LV Mass (g) and Biomarkers;
Archived Subjects

| | r value | p value |
|---|---|---|
| MMP 2 (ng/ml) | −0.045 | 0.592 |
| MMP 3 (ng/ml) | −0.103 | 0.220 |
| MMP 7 (ng/ml) | 0.121 | 0.147 |
| MMP 8 (ng/ml) | 0.183 | 0.028 |
| MMP 9 (ng/ml) | 0.497 | <0.001 |
| TIMP 1 (ng/ml) | 0.120 | 0.143 |
| TIMP 2 (ng/ml) | 0.298 | <0.001 |
| TIMP 4 (ng/ml) | 0.169 | 0.038 |
| PINP (ng/ml) | −0.156 | 0.060 |
| PIIINP (ng/ml) | 0.176 | 0.034 |
| CITP (ng/ml) | 0.149 | 0.073 |
| Cardiotrophin-1 (ng/ml) | 0.075 | 0.383 |
| sRAGE (ng/ml) | −0.147 | 0.076 |
| Osteopontin (ng/ml) | 0.178 | 0.033 |
| PIIINP:PINP Ratio | 0.292 | <0.001 |
| PINP:CITP Ratio | −0.122 | 0.145 |
| PIIINP:CITP Ratio | 0.045 | 0.594 |

Sample Size n = 150.

TABLE 23
Correlations for Relations between Arterial Systolic Pressure (mmHg)
and Biomarkers; Archived Subjects

| | r value | p value |
|---|---|---|
| MMP 2 (ng/ml) | −0.067 | 0.422 |
| MMP 3 (ng/ml) | −0.124 | 0.139 |
| MMP 7 (ng/ml) | 0.092 | 0.274 |
| MMP 8 (ng/ml) | 0.079 | 0.346 |
| MMP 9 (ng/ml) | 0.276 | <0.001 |
| TIMP 1 (ng/ml) | 0.199 | 0.015 |
| TIMP 2 (ng/ml) | 0.048 | 0.569 |
| TIMP 4 (ng/ml) | 0.112 | 0.175 |
| PINP (ng/ml) | −0.082 | 0.324 |
| PIIINP (ng/ml) | 0.173 | 0.037 |
| CITP (ng/ml) | −0.086 | 0.308 |
| Cardiotrophin-1 (ng/ml) | −0.016 | 0.857 |
| sRAGE (ng/ml) | −0.135 | 0.105 |
| Osteopontin (ng/ml) | 0.126 | 0.132 |
| PIIINP:PINP Ratio | 0.203 | 0.015 |
| PINP:CITP Ratio | 0.074 | 0.378 |
| PIIINP:CITP Ratio | 0.219 | 0.008 |

Sample Size n = 150.

TABLE 24
Changes from Normal Reference Group; Archived Subjects

| | LVH |
|---|---|
| MMP 2 (ng/ml) | [symbol] |
| MMP 3 (ng/ml) | ↓↓ |
| MMP 7 (ng/ml) | ↑↑ |
| MMP 8 (ng/ml) | ↑↑ |
| MMP 9 (ng/ml) | ↑↑ |
| TIMP 1 (ng/ml) | ↑↑ |
| TIMP 2 (ng/ml) | ↑↑ |
| TIMP 4 (ng/ml) | ↑↑ |
| PINP (ng/ml) | [symbol] |
| PIIINP (ng/ml) | ↑↑ |
| CITP (ng/ml) | [symbol] |
| Cardiotrophin-1 (ng/ml) | ↓ |
| sRAGE (ng/ml) | ↓ |
| Osteopontin (ng/ml) | ↑↑ |
| PIIINP:PINP Ratio | [symbol] |
| PINP:CITP Ratio | [symbol] |
| PIIINP:CITP Ratio | [symbol] |

Sample Size n = 150. [symbol]
= No Change
↑ = Significant Increase at p = 0.05-1.0
↓ = Significant Decrease at p = 0.05-1.0
↑↑ = Significant Increase at p < 0.05
↓↓ = Significant Decrease at p < 0.05

TABLE 25
Changes from LVH Only Group; Archived Subjects

| | CHF |
|---|---|
| MMP 2 (ng/ml) | ↑ |
| MMP 3 (ng/ml) | [symbol] |
| MMP 7 (ng/ml) | ↑↑ |
| MMP 8 (ng/ml) | [symbol] |
| MMP 9 (ng/ml) | [symbol] |
| TIMP 1 (ng/ml) | ↑↑ |
| TIMP 2 (ng/ml) | ↓ |
| TIMP 4 (ng/ml) | ↑↑ |
| PINP (ng/ml) | [symbol] |
| PIIINP (ng/ml) | ↑↑ |
| CITP (ng/ml) | [symbol] |
| Cardiotrophin-1 (ng/ml) | [symbol] |
| sRAGE (ng/ml) | ↑ |
| Osteopontin (ng/ml) | [symbol] |
| PIIINP:PINP Ratio | [symbol] |
| PINP:CITP Ratio | [symbol] |
| PIIINP:CITP Ratio | [symbol] |

Sample Size n = 150. [symbol]
= No Change
↑ = Significant Increase at p = 0.05-1.0
↓ = Significant Decrease at p = 0.05-1.0
↑↑ = Significant Increase at p < 0.05
↓↓ = Significant Decrease at p < 0.05

TABLE 26
LV Mass

| Dependent Variable | Parameter Estimate | Standard Error | p-value |
|---|---|---|---|
| Intercept | 2.33436 | 0.05383 | <0.0001 |
| NTBNP | 0.00019581 | 0.0006650 | 0.0038 |
| MMP-2 | −0.0006601 | 0.00013541 | <0.0001 |
| MMP-8 | 0.06363 | 0.02865 | 0.0281 |
| MMP-9 | 0.00204 | 0.00044689 | <0.0001 |
| MMP-9/TIMP-1 | −0.06094 | 0.02316 | 0.0095 |
| MMP-2 * gender | 0.00051455 | 0.00018204 | 0.0054 |
| MMP-8*gender | −0.08484 | 0.03645 | 0.0215 |
| Gender | −0.05809 | 0.07535 | 0.4421 |

Log10(LVMASS) = 2.33436 + 0.00019581 × AVE_BNP − 0.0006601 × MMP2 + 0.06363 × MMP8 + 0.00204 × MMP9 − 0.06094 × MMP9/timp1 + 0.005145 × male − 0.08484 × male − 0.05809 * male Adjusted r(sq) = 0.39

TABLE 27
LV Mass Index

| Dependent Variable | Parameter Estimate | Standard Error | p-value |
|---|---|---|---|
| Intercept | 1.87746 | 0.01899 | |
| NTBNP | 0.00025122 | 0.00005875 | <0.0001 |
| MMP-2 (centered) | −0.00046933 | 0.00012100 | 0.0002 |
| MMP-9 | 0.00073457 | 0.00017468 | <0.0001 |
| MMP-2 * gender | 0.00039964 | 0.00016209 | 0.015 |
| Gender | 0.02026 | 0.01884 | 0.28 |

Adjusted r(sq) = 0.29

TABLE 28
Pulmonary Capillary Wedge Pressure Estimates (PCWP)

| | | | |
|---|---|---|---|
| PIINP | 0.01422 | 0.00411 | 0.0008 |
| MMP3 | −0.00367 | 0.00176 | 0.0394 |
| MMP7 | 0.01831 | 0.00670 | 0.0072 |
| MMP8 | 0.04250 | 0.01989 | 0.0346 |

Log10 PCWP = 0.89286 + 0.01422 × PIINP − 0.00367 × MMP3 + 0.01831 × MMP7 + 0.04250 × MMP8 R(sq) = 0.21

TABLE 29
Six Minute Walk (r(sq) = 0.34)

| Dependent Variable | Parameter Estimate | Standard Error | p-value |
|---|---|---|---|
| Intercept | 428.6498 | 35.88661 | |
| Gender | −9.07044 | 31.58083 | 0.7748 |
| Mmp2 (centered) | 0.5209 | 0.19315 | 0.0088 |
| Mmp8 | −83.64827 | 30.77693 | 0.0083 |
| Mmp9 | −2.25501 | 0.62602 | 0.0006 |
| Mmp9:timp1 | 140.46835 | 31.53150 | <0.0001 |
| Mmp2 × gender | −0.65489 | 0.24412 | 0.0092 |

TABLE 30
Congestive Heart Failure

| Independent | Parameter | Standard | | 95% Wald |
|---|---|---|---|---|
| Variable | Estimate | Error | Odds Ratio | Confid. Limit |
| Intercept | −2.4060 | 0.5384 | | |
| MMP-7 | 0.3095 | 0.1187 | 1.363 | 1.08-1.72 |
| MMP-9 | 0.0253 | 0.00933 | 1.026 | 1.01-1.04 |
| MMP-9/TIMP-1 | −1.4669 | 0.6547 | 0.231 | 0.064-0.83 |
| Ratio | | | | |

C = 0.758

### REFERENCES

Bigg H F, Morrison C J, Butler G S, Bogoyevitch M A, Wang Z, Soloway P D, et al. Tissue inhibitor of metalloproteinase-4 inhibits but does not support the activation of gelatinase A via efficient inhibition of membrane type 1-matrix metalloproteinase. Cancer Res 2001; 61(9): 3610-8.
Bradham W S, Gunasinghe H, Holder J R, Multani M M, Killip D, Anderson M, et al. Release of matrix metalloproteinases following alcohol septal ablation in hypertrophic obstructive cardiomyopathy. JACC 2002; 40(12): 2165-73.
Brew K, Dinakarpandian D, Nagase H. Tissue inhibitors of metalloproteinases: evolution, structure and function. Biochimica et Biophysica Acta. 2000; 1477:267-283.
Caterina N C M, Windsor L J, Bodden M K, Yermovsky A E, Taylor K B, Birkendal-Hanson H, et al. Glycosylation and NH₂-terminal domain mutant of tissue inhibitor of metalloproteinases-1 (TIMP-1). Biochem Biophys Acta 1998; 1388: 21-34.
Chapman R E, Spinale F G. Extracellular protease activation and unraveling of the myocardial interstitium: critical steps toward clinical applications. Am J. Physiol. 2004; 286;H1-H10.
Chobanian A V, Bakris G L, Black H R, Cushman W C, Green L A, Izzo J L Jr, Jones D W, Materson B J, Oparil S, Wright J T Jr, Roccella E J; National Heart, Lung, and Blood Institute Joint National Committee on Prevention, Detection, Evaluation, and Treatment of High Blood Pressure; National High Blood Pressure Education Program Coordinating Committee. The Seventh Report of the Joint National Committee on Prevention, Detection, Evaluation, and Treatment of High Blood Pressure: the JNC 7 report. JAMA. 2003; 289:2560-72.
Creemers E E J M, Cleutjens J P M, Smits J F M, Daemen M J A P. Matrix metalloproteinase inhibition after myocardial infarction. A new approach to prevent heart failure? Circulation Res 2001; 89; 201-210.
Dennis J W, Granovsky M, Warren C E. Protein glycosylation in development and disease. BioEssays 1999; 21: 412-421.
Deschamps A M, Apple K A, Leonardi A H, McLean J E, Yarbrough W M, Stroud R E, Clark L L, Sample J A, Spinale F G. Myocardial interstitial matrix metalloproteinase activity is altered by mechanical changes in LV load: interaction with the angiotensin type 1 receptor. Circ Res. 2005; 27; 96:1110-8.
Devereux R B, Alonso D R, Lutas E M, Gottlieb G J, Campo E, Sachs I, Reichek N. Echocardiographic assessment of left ventricular hypertrophy: comparison to necropsy findings. Am J. Cardiol. 1986; 57:450-8.
Diez J, Querejeta R, Lopez B, Gonzalez A, Larman M, Martinez Ubago J L. Losartan-dependent regression of myocardial fibrosis is associated with reduction of left ventricular chamber stiffness in hypertensive patients. Circulation. 2002; 105:2512-2517.
Douglas D A, Shi E, Sang Q A. Computational sequence analysis of the tissue inhibitor of metalloproteinase family. J Protein Chem 1997, 16: 237-255.
Edwards D R, Beaudry P P, Laing T D, Kowal V, Leco K J, Leco P A, Lim M S. The roles of tissue inhibitors of metalloproteinases in tissue remodeling and cell growth. Int J Obes 1996: 20: S9-S15.
Galis Z S, Khatri J J. Matrix metalloproteinases in vascular remodeling and atherogenesis: the good, the bad and the ugly. Circ Res 2002; 90: 251-62.
Goffin F, Munaut C, Frankenne F, Perrier D'Hauterive S, Beliard A, Fridman V, et al. Expression pattern of metalloproteinases and tissue inhibitor of matrix metalloproteinases in cycling human endometrium. Biol Reprod 2003.
Gomez D E, Alonso D F, Yoshiji H, Thogeirsson U P. Tissue inhibitor of metalloproteinases: structure, regulation, and biological functions. EJCB 1997, 74: 111-112.
Greene J, Wang M, Liu Y E, Raymond L A, Rosen C, Shi Y E. Molecular cloning and characterization of human tissue inhibitor of metalloproteinase 4. J Biol Chem 1996; 271: 30375-30380.
Gunasinghe S K, Ikonomidis J S, Spinale F G. Contributory role of matrix metalloproteinases in cardiovascular remodeling. Cardiovasc Heamat Disorders, 1(2) 75-91, 2001 Nagase H. Activational mechanisms of matrix metalloproteinases. Biological Chemistry 1997; 378: 151-160.
Hojo Y, Ikeda U, Ueno S, Arakawa H, Shimada K. Expression of matrix metalloproteinases in patients with acute myocardial infarction. Jpn Circ J 2001; 65: 71-75.
Joffs C, Himali R, Gunasinghe R S, Multani M M, Dorman B H, Kratz J M, Crumbley A J III, Crawford F A Jr., Spinale F G. Cardiopulmonary bypass induces the synthesis and release of matrix metalloproteinases. Ann Thorac Surg. 2001; 71:1518-23.
Kai H, Ikeda H, Yusakawa H, Kai M, Seki Y, Kuwahara F, Ueno T, Sugi K, Imaizumi T. Peripheral blood levels of matrix metalloproteinases-2 and -9 are elevated in patients with acute coronary syndromes. J Am Coll Cardiol 1998; 32: 368-372.
Kenchaiah S, Pfeffer M A. Cardiac remodeling in systemic hypertension. Med Clin North Am. 2004; 88:115-130.
Laviades C, Varo N, Fernandes J, Mayor G, Gil M J, Monreal I, Diez J. Abnormalities of the extracellular degradation of collagen type I in essential hypertension. Circulation. 1998; 98:535-540.
Levy D, Larson M G, Vasan R S, Kannel W B, Ho K K L: The progression from hypertension to congestive heart failure. JAMA. 1996; 275: 1557-1562.
Li Y Y, Feldman A M, Sun Y, McTiernan C F. Differential expression of tissue inhibitors of metalloproteinases in the failing human heart. Circulation 1998, 98: 1728-1734.
Li Y Y, Feng, McTierman C F, Pei W, Moravec C S, Wang P, et al. Downregulation of matrix metalloproteinases and reduction in collagen damage in the failing human heart after support with left ventricular assist devices. Circulation 2001; 104: 1147-52.
Lindsay M M, Maxwell P, Dunn F G: TIMP-1. A marker of left ventricular diastolic dysfunction and fibrosis in hypertension. Hypertension. 2002; 40:136-141.
Lindsey M L, Mann D L, Entman M L, Spinale F G. Extracellular matrix remodeling following myocardial injury. Ann Med. 2003; 35:316-316.
Li-Saw-Hee F L, Edmunds E, Blann A D, Beevers D G, Lip G Y H: Matrix metalloproteinase-9 and tissue inhibitor metalloproteinase-1 levels in essential hypertension. Relationship to left ventricular mass and anti-hypertensive therapy. Int J Cardiol. 2000; 75:43-47.
Liu Y E, Wang M, Greene J, Su J, Ullrich S, Li H, Sheng S, Alexander P, Sang Q A, Shi Y E. Preparation and characterization of recombinant tissue inhibitor of metalloproteinase 4. Am Soc Biochem Mol Biol 1997, 272: 20479-20483.
Lloyd-Jones D M, Larson M G, Leip E P, Beiser A, D'Agostino R B, Kannel W B, Murabito J M, Vasan R S, Benjamin E J, Levy D. Lifetime risk for developing congestive heart failure. The Framingham Study. Circulation. 2002; 106:3068-3072.
Lopez B, Gonzalez A, Varo N, Laviades C, Querejeta R, Diez: J: Biochemical assessment of myocardial fibrosis in hypertensive heart disease. Hypertension. 2001b; 38:1222-1226.
Lopez B, Querejeta R, Varo N, Gonzalez A, Larman M, Ubago J L M, Diez J: Usefulness of serum carboxy-terminal propeptide of procollagen type I in assessment of the cardioreparative ability in antihypertensive treatment in hypertensive patients. Circulation. 2001a; 104:286-291.
Maron B J. Hypertrophic cardiomyopathy: a systematic review. JAMA 2002; 13: 287(1308-1320).
Nagueh S F, Lakkis N M, Middleton K J, Killip D, Zoghbi W A, Quinones M A, Spencer S H 3rd. Changes in left ventricular diastolic function 6 months after nonsurgical septal reduction therapy for hypertrophic obstructive cardiomyopathy. Circulation 1999: 99:344-347.
Nagueh S F, Lakkis N M, Middleton K J, Killip D, Zoghbi W A, Quinones M A, Spencer S H 3rd. Changes in left ventricular filling and left atrial function six months after nonsurgical septal reduction therapy for hypertrophic obstructive cardiomyopathy. J Am Col1Cardiol 1999; 34: 1123-1128.
Nagueh S F, Mikati I, Kopelen H A, Middleton K J, Quinones M A, Zoghbi W A: Doppler estimation of left ventricular filling pressure in sinus tachycardia. A new application of tissue Doppler imaging. Circulation. 1998; 98:1644-1650.
Nuegh S F, Stevenson S J, Lakkis N M, Killip D, Perez-Verdia A, Entman M L, et al. Decreased expression of tumor necrosis factor-alpha and regression of hypertrophy after nonsurgical septal reduction therapy for patients with hypertrophic obstructive cardiomyopathy. Circulation 2001; 103(14): 1844-50.
Parsons S L, Watson S A, Brown P D, Collins H M, Steele R J C. Matrix metalloproteinases. Brit J Surg 1997; 84:160-166.
Peterson J T, Li H, Dilon L, Bryant J W. Evolution of matrix metalloproteinase and tissue inhibitor expression during heart failure progression in the infracted rat. Cardiovas Res 2000; 46: 307-315.
Radomski A, Juraz P, Sanders E J, Overall C M, Biggs H F, Edwards D R, et al. Identification, regulation and role of tissue of tissue inhibitor of metalloproteinases-4 (TIMP-4) in human platelets. Br J Pharmaco 2002; 137(8): 1130-1338.
Sahn D J, DeMaria A, Kisslo J, Weyman A. Recommendations regarding quantitation in M-mode echocardiography: results of a survey of echocardiographic measurements. Circulation. 1978; 58: 1072-1083.
Schillaci G. Pasqualini L, Verdecchia P, Vaudo G, Marchesi S, Porcellati C, De Simone G, Mannarion E. Prognostic significance of left ventricular diastolic dysfunction in essential hypertension. J Am Col1 Cardiol. 2002; 39:2005-2011.
Schiller N B, Shah P M, Crawford M, DeMaria A, Devereux R, Feigenbaum H, Gutgesell H, Reichek N, Sahn D, Schnittger I. Recommendations for quantitation of the left ventricle by two-dimensional echocardiography. American Society of Echocardiography Committee on Standards, Subcommittee on Quantitation of Two-Dimensional Echocardiograms. J Am Soc Echocardiography. 1989; 2: 358-367.
Sharp P S, Rainbow S, Mukherjee S. Serum levels of low molecular weight advanced glycation end products in diabetic subjects. Diabet Med 2003; 20(7): 575-9.
Spencer W H 3rd, Roberts R. Alcohol septal ablation in hypertrophic obstructive cardiomyopathy: the need for a registry. Circulation 2000; 102: 600-01.
Spinale F G, Coker M L, Heung L J, Bond B R, Gunasinghe H R, Etoh T, et al. A matrix metalloproteinase induction/activation system exists in the human left ventricular myocardium and is upregulated in heart failure. Circulation 2000; 102; 1944-1949.
Spinale, F G. Matrix metalloproteinases. Regulation and dysregulation in the failing heart. Circ. Res. 2002; 90:520-530.
Steinberg T H, Pretty On Top K, Berggren K N, Kemper C, Jones L, Diwu Z, et al. Rapid and simple single nanogram detection of glycoproteins in polyacrylamide gels on electroblots. Proteomics 2001; 1(7): 841-55.
Stroud R E, Deschamps A M, Lowry A S, Hardin A E, Mukherjee R, Lindsey M L, Ramamoorthy S, Zile M R, Spencer W H, Spinale F G. Plasma monitoring of the myocardial specific tissue inhibitor of metalloproteinase-4 after alcohol septal ablation in hypertrophic obstructive cardiomyopathy. J Card Fail. 2005; 11:124-30
Tayebjee M H, Lim H S, Nadar S, MacFadyen R J, Lip G Y. Tissue inhibitor of metalloproteinse-1 is a marker of diastolic dysfunction using tissue doppler in patients with type 2 diabetes and hypertension. Eur J Clin Invest. 2005; 35:8-12.
Tayebjee M H, Nadar S, Blann A D, Gareth Beevers D, MacFadyen R J, Lip G Y. Matrix metalloproteinase-9 and tissue inhibitor of metalloproteinase-1 in hypertension and their relationship to cardiovascular risk and treatment: a substudy of the Anglo-Scandinavian Cardiac Outcomes Trial (ASCOT). Am J. Hypertens. 2004; 17:764-9.
Tayebjee M H, Nadar S K, MacFadyen R J, Lip G Y. Tissue inhibitor of metalloproteinase-1 and matrix metalloproteinase-9 levels in patients with hypertension Relationship to tissue Doppler indices of diastolic relaxation. Am J. Hypertens. 2004; 17:770-4.
Timms, P M, Wright A, Maxwell P, Campbell S, Dawnay A B, Srikanthan V. Plasma tissue inhibitor of metalloproteinase-1 levels are elevated in essential hypertension and related to left ventricular hypertrophy. Am J. Hyper. 2002:15:269-272.
Tsuruda T, Costello-Boerrigter L C, Burnett J C Jr. Matrix metalloproteinases: pathways of induction by bioactive molecules. Heart Fail Rev. 2004; 9:53-61.
Vu T H, Werb Z. Matrix metalloproteinases: effectors of development and normal physiology. Genes Dev 2000; 14:2123-2133Gross J, Lapiere C M. Collagenolytic activity in amphibian tissues: a tissue culture assay. Proc Natl Acad Sci USA 1962; 48: 1014-1022.
Wachtell, K, Smith G, Gerdts E, Dahlof B, Nieminen M S, Papademetriou V, Bella J N, Ibsen H, Rokkedal J, Devereux R B. Left ventricular filling patterns in patients with systemic hypertension and left ventricular hypertrophy (The Life Study). Am J. Cardiol. 2000; 85:466-472.
Wassef M, Baxter B T, Chisholm R L, Dalman R L, Fillinger M F, Heinecke J, et al. Pathogenesis of abdominal aortic aneurysms: a multidisciplinary research program supported by the National Heart, Lung, and Blood Institute. J Vas Surg 2001; 34: 730-8.
Weber K T, Brilla C G. Pathological hypertrophy and cardiac interstitium. Fibrosis and renin-angiotensin-aldosterone system. Circulation 1991; 83: 1849-65.
Weber K T, Sun Y, Guarda E: Structural remodeling in hypertensive heart disease and the role of hormones. Hypertension. 1994; 23:869-877.
Wilson E M, Gunasinghe H R, Coker M L, Sprunger P, Lee-Jackson D, Bozkurt B, Deswal A, Mann D L, Spinale F G. Plasma matrix metalloproteinase and inhibitor profiles in patients with heart failure. J Cardiac Failure. 2002; 8:390-398.
Woessner J F, Nagase H. Activation of the zymogen forms of MMPs. In: Matrix metalloproteinases and TIMPs. Oxford University Press, Oxford U K, 2000 pp 72-86.
Yarbrough W M, Mukherjee R, Escobar P, Mingoia J T, Sample J A, Hendrick J W, Dowdy K B, McLean J E, Lowry A S, O'Neil T P, Spinale F G. Selective targeting and timing of matrix metalloproteinase inhibition in post-myocardial infarction remodeling. Circulation. 2003; 108:1753-1759.
Yasmin, Wallace S, McEniery C M, Dakham Z, Pusalkar P, Maki-Petaja K, Ashby M J, Cockcroft J R, Wilkinson I B. Matrix metalloproteinase-9 (MMP-9), MMP-2, and serum elastase activity are associated with systolic hypertension and arterial stiffness. Arterioscler Thromb Vasc Biol. 2005; 25:372.
Zervoudaki A, Economou E, Stefanadis C, Pitsavos C, Tsioufis K, Aggeli C, Vasiliadou K, Toutouza M, Toutouzas P: Plasma levels of active extracellular matrix metalloproteinases 2 and 9 in patients with essential hypertension before and after antihypertensive treatment. J Hum Hypertens. 2003; 17:119-124.
Zile M R, Brutsaert D L: New concepts in diastolic dysfunction and diastolic heart failure. Part I: Diagnosis, prognosis, measurements of diastolic function. Circulation. 2002; 105:1487-1393.
Zile M R, Brutsaert D L: New concepts in diastolic dysfunction and diastolic heart failure. Part II: Causal mechanisms and treatment. Circulation. 2002; 105:1503-1508.

1. A method comprising measuring a biomarker profile, wherein the biomarker profile comprises the amount of four or more biomarkers in a body fluid from a subject, wherein the amount of four or more of the four or more biomarkers compared to a reference amount for the biomarker indicates the presence or risk of diastolic heart failure in the subject, wherein the four or more biomarkers comprise propeptide for collagen III (PIIINP), MMP-2, and TIMP-4,
wherein an amount of PIIINP that is greater than the reference amount, an amount of MMP-2 that is greater than the reference amount, an amount of TIMP-4 that is greater than the reference amount, or a combination indicates the presence or risk of diastolic heart failure in the subject.

2-3. (canceled)

4. The method of claim 1, wherein the amount of PIIINP in a body fluid from the subject is measured, wherein the reference amount is the amount in a normal subject, wherein an amount of PIIINP that is greater than the reference amount indicates the presence or risk of diastolic heart failure in the subject.

5. The method of claim 4, wherein the amount of PIIINP is at least about 10% greater than the reference amount.

6-11. (canceled)

12. The method of claim 1, wherein the amount of MMP-2 in a body fluid from the subject is measured, wherein the reference amount is the amount in a subject with left ventricular hypertrophy, wherein an amount of MMP-2 that is greater than the reference amount indicates the presence or risk of diastolic heart failure in the subject.

13. The method of claim 12, wherein the amount of MMP-2 is at least about 10% greater than the reference amount.

14. The method of claim 1, wherein the amount of MMP-2 in a body fluid from the subject is measured, wherein the reference amount is the amount in a normal subject, wherein an amount of MMP-2 that is greater than the reference amount indicates the presence or risk of diastolic heart failure in the subject.

15. The method of claim 14, wherein the amount of MMP-2 is at least about 10% greater than the reference amount.

16-30. (canceled)

31. The method of claim 1, wherein the amount of TIMP-4 in a body fluid from the subject is measured, wherein the reference amount is the amount in a normal subject, wherein an amount of TIMP-4 that is greater than the reference amount indicates the presence or risk of diastolic heart failure in the subject.

32. The method of claim 31, wherein the amount of TIMP-4 is at least about 50% greater than the reference amount.

33-81. (canceled)

82. A method comprising measuring a biomarker profile, wherein the biomarker profile comprises the amount of three or more biomarkers in a body fluid from a subject, wherein the amount of three or more of the three or more biomarkers compared to a reference amount for the biomarker indicates the presence or risk of left ventricular hypertrophy in the subject, wherein the three or more biomarkers comprise propeptide for collagen III (PIIINP), MMP-9, and TIMP-1,
wherein an amount of PIIINP that is greater than the reference amount, an amount of MMP-9 that is greater than the reference amount, an amount of TIMP-1 that is greater than the reference amount, or a combination indicates the presence or risk of left ventricular hypertrophy in the subject.

83-84. (canceled)

85. The method of claim 1, wherein the body fluid is blood.

86. The method of claim 1, wherein the body fluid is plasma.

87. (canceled)

88. The method of claim 1, wherein the amount of four or more of the two or more biomarkers is measured.

89. The method of claim 1, wherein the amount of five or more of the two or more biomarkers is measured.

90-94. (canceled)

95. The method of claim 82, wherein the three or more biomarkers further comprise MMP-7, N-terminal pro-B-type natriuretic peptide (NTBNP), or both.

96. The method of claim 95, wherein an amount of MMP-7 that is greater than the reference amount, an amount of NTBNP that is greater than the reference amount, or a combination indicates the presence or risk of left ventricular hypertrophy in the subject.
